(19) United States
(12) Patent Application Publication
(10) Pub. No.: US 2021/0333275 A1
(43) Pub. Date: Oct. 28, 2021

(54) PAN-ISOTYPE IMMUNOGLOBULIN FINGERPRINTING

(71) Applicant: CDI Laboratories, Inc., Mayagüez, PR (US)

(72) Inventor: Tyler HULETT, Portland, OR (US)

(21) Appl. No.: 17/208,878

(22) Filed: Mar. 22, 2021

Related U.S. Application Data
(60) Provisional application No. 62/993,673, filed on Mar. 23, 2020.

(51) Int. Cl. G01N 33/564

(52) U.S. Cl. CPC G01N 33/564

(57) ABSTRACT

Disclosed herein are methods and compositions for classifying individual antigen-specific antibody binding. Disclosed herein are methods and compositions for a proteome-wide pan-isotype immunoassay. Disclosed herein are methods and compositions for immunoglobulin fingerprinting.

### CROSS-REFERENCE

This application claims the benefit of U.S. Provisional Application No. 62/993,673, filed Mar. 23, 2020, which application is incorporated herein by reference.

### SUMMARY OF THE INVENTION

Disclosed herein are methods of pan-isotype immunoglobulin fingerprinting. In some embodiments, a method can comprise contacting an array with a sample; detecting a first antibody isotype and a second antibody isotype in a sample, and computing a ratio of a first antibody isotype to a second antibody isotype. In some embodiments, an array can comprise at least one native folded human protein or an antigenic fragment thereof. In some embodiments, an array can comprise a virome, allergome, mutanome, microbiome, or phageome. In some embodiments, a first antibody isotype can comprise an antibody subclass, a second antibody isotype can comprise an antibody subclass or a first antibody isotype and a second antibody isotype can comprise an antibody subclass. In some embodiments, a first antibody isotype or a second antibody isotype can comprise IgM, IgM B-cell receptor, IgM pentamer, IgG, IgG1, IgG2, IgG3, IgG4, IgA, IgA1, IgA1 monomer, IgA1 dimer, secreted IgA1 dimer, IgA2, IgA2 monomer, secreted IgA2 dimer, IgD, IgD B-cell receptor, IgE, or any combination thereof. In some embodiments, a first antibody isotype or a second antibody isotype can comprise an IgG. In some embodiments, an IgG can be of an antibody subclass comprising IgG1, IgG2, IgG3 or IgG4. In some embodiments, an IgG can be of an antibody subclass comprising an IgG2, wherein an IgG2 can comprise IgG2A, IgG2B, or IgG2C. In some embodiments, an array can comprise at least about 10, 50, 100, 1000 native folding human proteins or an antigenic fragment thereof. In some embodiments, a sample can comprise blood, serum, urine or a combination thereof. In some embodiments, a sample can be from a human subject. In some embodiments, a detecting of a first antibody isotype and a second antibody isotype in a sample can comprise staining of a first antibody isotype and a second antibody isotype. In some embodiments, a first antibody isotype can be stained with a first stain and a second antibody isotype can be stained with a different second stain. In some embodiments, a first stain can comprise anti-IgM, anti-IgM B-cell receptor, anti-IgM pentamer, anti-IgG, anti-IgG1, anti-IgG2, anti-IgG3, anti-IgG4, anti-IgA, anti-IgA1, anti-IgA1 monomer, anti-IgA1 dimer, anti-secreted IgA1 dimer, anti-IgA2, anti-IgA2 monomer, anti-secreted IgA2 dimer, anti-IgD, anti-IgD B-cell receptor, anti-IgE, or any combination thereof. In some embodiments, a second stain can comprise anti-IgM, anti-IgM B-cell receptor, anti-IgM pentamer, anti-IgG, anti-IgG1, anti-IgG2, anti-IgG3, anti-IgG4, anti-IgA, anti-IgA1, anti-IgA1 monomer, anti-IgA1 dimer, anti-secreted IgA1 dimer, anti-IgA2, anti-IgA2 monomer, anti-secreted IgA2 dimer, anti-IgD, anti-IgD B-cell receptor, anti-IgE, or any combination thereof. In some embodiments, a staining comprise a fluorescent staining, and a detecting can comprise detecting a fluorescent staining. In some embodiments, detecting a fluorescent staining can comprise scanning an array with a scanner. In some embodiments, detecting can comprise a multicolor detection. In some embodiments, a detecting can comprise a three-dimensional detection. In some embodiments, a detecting can comprise a pull down assay. In some embodiments, a pull down assay can comprise isolating a protein that binds to a bait. In some embodiments, a detecting can comprise sequencing of an isolated protein. In some embodiments, a detecting can comprise phage immunoprecipitation sequencing (PhIP-seq), Rapid Extracellular Antigen Profiling (REAP), mass spectrometry, protein microarrays, peptide microarrays, Luminex libraries, SEREX, nucleotide barcode labelled peptide sequencing, or any combination thereof. In some embodiments, a data from a detection can be transmitted to a computer configured to calculate a ratio between a first antibody isotype and a second antibody isotype. In some embodiments, a ratio can be used to determine a disease target, a disease risk, a treatment response, or a combination thereof. In some embodiments, determining a disease target can comprise identifying a novel treatment target. In some embodiments, a treatment can comprise a drug. In some embodiments, a disease risk can comprise a risk of a subject developing a chronic disease. In some embodiments, determining a treatment response, can comprise stratifying patients into high responders, medium responders, and low responders to treatment. In some embodiments, an array can comprise at least about 10,000, 15,000, or 20,000 unique human proteins. In some embodiments, unique human proteins comprise at least 80%, 90% or 100% full-length proteins. In some embodiments, a method can further comprise combining a ratio with PhIP-Seq data. In some embodiments, PhIP-Seq data can comprise: human proteome, virome, allergome, mutanome, microbiome, phageome, retrovirome, common polymorphisms, or any combination thereof. In some embodiments, a method can further comprise generating an integrated readout of isotype-ratios with epitope-level information. In some embodiments, epitope-level information can be derived from phage-display immunoprecipitation sequencing (PhIP-Seq) for a related protein library. In some embodiments, a method can further comprise combining a ratio with ‘BLAST’ type cross comparisons, wherein combining allows for correlating cross reactive antigens from their origin in virus or microbe to isotype-ratio immune response. In some embodiments, a method can further comprise combining a ratio with a patient-specific multi-omic integration. In some embodiments, a patient-specific multi-omic integration can comprise: HLA-binding algorithms, genomic sequencing, T cell receptor/B cell receptor sequencing, or any combination thereof. In some embodiments, a T cell receptor/B cell receptor sequencing can predict likelihood of corresponding T cell responses based on a ratio and determining an individual parent cell clone. In some embodiments, a method can further comprise combining a ratio with known ontology databases. In some embodiments, a first antibody isotype or a second antibody isotype can be identified as negative, mid, or high. In some embodiments, a ratio can be indicative of an antigen-specific antibody response. In some embodiments, an antigen-specific antibody response can comprise pro-tolerance, pro-effector, anti-allergen, pro-IgG4-specific disease, anti-viral or a combination thereof. In some embodiments, a method can be used for diagnosing, stratifying, or treating a disease or condition. In some embodiments, a disease or condition can comprise cancer, an autoimmune disease, a neurodegenerative disease, autism, epilepsy, schizophrenia, healthy ageing, unhealthy ageing, cardiovascular disease, obesity, diabetes, COVID-19, HIV, food intolerances, allergies, bacterial infections, or a combination thereof. In some embodiments, a ratio can be indicative of a response to a treatment. In some embodiments, a treatment can comprise antigen-specific CAR-T cell therapy, antigen-specific CAR-B cell therapy, monoclonal antibodies and antibody derivatives, BiTEs, DARTs, tolerogenic vaccines, effector vaccines, gene therapy or a combination thereof. In some embodiments, a first antibody isotype and a second antibody isotype are different. In some embodiments, a first antibody isotype can comprise at least 2, 3, 4, 5, or 10 antibody isotypes. In some embodiments, a second antibody isotype can comprise at least 2, 3, 4, 5, or 10 antibody isotypes. In some embodiments, a detecting can be performed at a first time point and a second time point. In some embodiments, a protein or moiety on an array can comprise a modification. In some embodiments, an array can comprise a pan modification. In some embodiments, a modification can comprise acetylation, acylation, adenylylation, amidation, arginylation, biotinylation, carbamylation, carbonylation, carboxylation, citrullination, eliminylation, farnesylation, formylation, glycation, glycosylation, glypiation, hydroxylation, imination, isoprenylation, lipidation, lipoylation, malonylation, methylation, myristoylation, neddylation, nitrosylation, oxidation, palmitoylation, pegylation, phophopantetheinylation, phosphorylation, polyglutamylation, prenylation, pupylation, succinylation, sulfation, sumoylation, ubiquitylation, or any combination thereof. In some embodiments, a ratio can comprise a ratio of IgG1/IgG3, IgG1/IgA2, or IgG4/IgE. In some embodiments, a first antibody isotype, a second antibody isotype, or both can comprise an autoantibody. In some embodiments, a pattern of correlation can be determined to comprise a ratio of a first antibody isotype to a second antibody isotype. In some embodiments, a pattern can be transmitted to a computer, wherein a computer comprises a CPU, and at least one memory interfaced with a CPU, wherein a data generated from a method can be processed, analyzed or stored on a computer. In some embodiments, a pattern can be used at least in part to determine a treatment regimen. Disclosed herein in some embodiments, is a system comprising an antigen array, a detection apparatus, and a computer configured to obtain and process data from a detection apparatus, wherein a data comprises a ratio of a first antibody to a second antibody detected on an antigen array. In some embodiments, a system can be configured to perform a method of as disclosed herein.

Also disclosed herein is a method of detecting an antibody isotype pattern on an array, a method comprising contacting an array with a sample; and detecting a first antibody isotype and a second antibody isotype in a sample, wherein a first antibody isotype and a second antibody isotype generate a pattern on an array. In some embodiments, a method can further comprise detecting a disease based at least in part on a pattern on an array. In some embodiments, a method can further comprise detecting a stage of a disease based at least in part on a pattern on an array. In some embodiments, multiple patterns can be detected. In some embodiments, a method can further comprise use of a computer comprising a CPU, at least one memory interfaced with said CPU, wherein data generated from a method can be processed, analyzed or stored on a computer. In some embodiments, machine learning can be used to cluster proteome-wide pan-isotype autoantibody immunoprofiling data. In some embodiments, a clustering can be performed according to a multidimensional population-wide antigen-specific interrelationships across immunoglobulin isotypes and subtypes. In some embodiments, a choice of treatment can be based at least in part on a method disclosed herein. Disclosed herein in some embodiments, is a system comprising an antigen array, a detection apparatus, and a computer configured to obtain and process data from a detection apparatus, wherein a data comprises a ratio of a first antibody to a second antibody detected on an antigen array. In some embodiments, a system can be configured to perform a method of as disclosed herein.

Disclosed herein in some embodiments, is a system comprising an antigen array, a detection apparatus, and a computer configured to obtain and process data from a detection apparatus, wherein a data comprises a ratio of a first antibody to a second antibody detected on an antigen array. In some embodiments, a first antibody isotype, a second antibody isotype, or both can comprise an autoantibody. In some embodiments, a pattern of correlation can be determined comprising a ratio of a first antibody isotype to a second antibody isotype. In some embodiments, a detection apparatus can comprise a scanner configured to detect a staining of a first antibody isotype and a second antibody isotype, a sequencing apparatus configured to sequence a first antibody isotype and a second antibody isotype, or any combination thereof. In some embodiments, a system can be configured to perform a method of as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts proteome-wide pan-isotype autoantibody immunoprofiling of a healthy human male. The data demonstrates population-wide antigen-specific interrelationships across immunoglobulin isotypes and subtypes.

FIG. 2 depicts reproducible proteome-wide IgG autoantibody immunoprofiling of a healthy human male within and across human proteome microarray batches demonstrates reliability of multi-array analysis.

FIG. 3 depicts reproducible proteome-wide IgA autoantibody immunoprofiling of a healthy human male within and across human proteome microarray batches demonstrates reliability of multi-array analysis.

FIG. 4 depicts a demonstration of differential antibody affinity populations by proteome array titration in serum from a healthy human male. FIG. 4A depicts the data for IgG. FIG. 4B depicts the data for IgA.

FIG. 5 depicts the flow of combined data sources used in the generation of the combined multi-dimensional readout.

FIG. 6 depicts an increase in PubMed publications over time including the term ‘inflammatory’ in the abstract across: FIG. 6A all different disease conditions, and also in individual categories including the words: FIG. 6B microbiome, FIG. 6C autism, FIG. 6D cardiovascular, FIG. 6E aging, FIG. 6F cancer, FIG. 6G neurodegenerative, FIG. 6H mental illness, FIG. 6I Alzheimer's.

FIG. 7 shows some of the known function and diversity of individual antibody isotypes and subtypes.

FIG. 8 depicts a graph showing the numbers of proteins of each category in the array.

FIG. 9 depicts an image showing a more precise spot morphology produced by a manufacturing method using a non-contact piezoelectric printer compared to a contact array printer.

FIG. 10 depicts a schematic outlining a method of using a human proteome microarray for antibody screening.

FIG. 11 depicts a schematic outlining a method of using a human proteome microarray for antibody serum screening at an epitope level and subsequent data analysis.

FIG. 12 depicts a schematic outlining a method of using a human virome microarray for antibody serum screening at an epitope level and subsequent data analysis.

FIG. 13 shows a computer control system that may be programmed or otherwise configured to implement methods provided herein.

FIG. 14 depicts a schematic outlining a method of creating a human proteome microarray for antibody screening.

FIG. 15 depicts a schematic outlining a method of using a human proteome microarray for antibody screening.

FIG. 16 depicts a schematic outlining a method of using a human virome microarray for antibody serum screening at an epitope level and subsequent data analysis.

FIG. 17 depicts a schematic outlining a method of using a human proteome microarray for antibody serum screening at an epitope level and subsequent data analysis.

FIG. 18 depicts a schematic outlining a method of using a xenoantigen proteome microarray for antibody serum screening at an epitope level and subsequent data analysis.

FIG. 19 shows output from a computer control system that uses machine learning to cluster proteome-wide pan-isotype autoantibody immunoprofiling data from a healthy human male according to multidimensional population-wide antigen-specific interrelationships across immunoglobulin isotypes and subtypes.

FIG. 20 shows output from a computer control system that uses machine learning to cluster proteome-wide pan-isotype autoantibody immunoprofiling data from a healthy human male according to multidimensional population-wide antigen-specific interrelationships across immunoglobulin isotypes and subtypes filtered to demonstrate this output associates with gene ontology categories of interest—in this case druggable GPCRs.

FIG. 21 shows correlation coefficients of each individual isotype ratio pair included in the analysis demonstrated some isotypes were more closely linked to one another than others.

FIG. 22 shows individual plots of log 10-transformed isotype-pair comparisons used to compute the correlation in FIG. 20.

FIG. 23 depicts raw log 2 transformed proteome-wide pan-isotype autoantibody immunoprofiling of a child with Systemic lupus erythematosus (SLE). The data demonstrates unique population-wide antigen-specific interrelationships across immunoglobulin isotypes and subtypes associated with lupus.

FIG. 24A depicts the utility of this invention via raw log 2 transformed proteome-wide pan-isotype autoantibody immunoprofiling of a child with Systemic lupus erythematosus (SLE). FIG. 24B and FIG. 24C show the known lupus autoantigens Sm/RNP Complex, SM Antigen, and SNRPB are contained in the ‘IgG1high IgG2high’ autoantibody staining compartment. The autoantigens in this compartment are significantly enriched by gene ontology enrichment analysis for antigens from TGF-beta signaling cytokines and molecules, messenger RNA processing, capped intron-containing pre-mRNA processing, messenger RNA splicing, major pathway, and myogenesis; pathways known to behave aberrantly in lupus. FIG. 24D and FIG. 24G show that when filtering for additional isotypes, these enrichments isolate to specific multi-isotype sub-compartments. FIG. 24E and FIG. 24F show that TGF-beta signaling cytokines and molecules and myogenesis-associated biomarkers are contained within the ‘IgG1high IgG2high IgG4negative IgEnegative’ sub-compartment. FIG. 24H show that the mRNA associated messenger RNA processing, capped intron-containing pre-mRNA processing, and messenger RNA splicing, major pathway biomarkers are contained within the ‘IgG1high IgG2high IgGA2positive’ sub-compartment.

FIG. 25 depicts raw log 2 transformed proteome-wide pan-isotype autoantibody immunoprofiling of a healthy child. The data demonstrates unique population-wide antigen-specific interrelationships across immunoglobulin isotypes and subtypes.

FIG. 26 depicts raw log 2 transformed proteome-wide pan-isotype autoantibody immunoprofiling of a healthy adult. The data demonstrates unique population-wide antigen-specific interrelationships across immunoglobulin isotypes and subtypes.

FIG. 27 depicts raw log 2 transformed proteome-wide pan-isotype autoantibody immunoprofiling of the previous healthy adult, one year later and recently recovered from viral shingles and COVID-19 vaccination. The data demonstrates unique population-wide antigen-specific interrelationships across immunoglobulin isotypes and subtypes that either change or are stable with time and are associated with viral shingles and COVID-19 vaccination.

FIG. 28 depicts raw log 2 transformed proteome-wide pan-isotype autoantibody immunoprofiling of a child with COVID-19-induced MISC autoimmune disease. The data demonstrates unique population-wide antigen-specific interrelationships across immunoglobulin isotypes and subtypes associated with COVID-19-induced MISC autoimmune disease.

FIG. 29 depicts raw log 2 transformed proteome-wide pan-isotype autoantibody immunoprofiling of a patient with CTLA-4-knockout genetically inherited immune disease. The data demonstrates unique population-wide antigen-specific interrelationships across immunoglobulin isotypes and subtypes associated with CTLA-4-knockout genetically inherited immune disease.

FIG. 30 depicts raw log 2 transformed proteome-wide pan-isotype autoantibody immunoprofiling of a patient with cancerous lymphoma. The data demonstrates unique population-wide antigen-specific interrelationships across immunoglobulin isotypes and subtypes associated with cancerous lymphoma.

FIG. 31 depicts raw log 2 transformed proteome-wide pan-isotype autoantibody immunoprofiling of a patient with ulcerative colitis. The data demonstrates unique population-wide antigen-specific interrelationships across immunoglobulin isotypes and subtypes associated with ulcerative colitis.

FIG. 32 depicts the utility of this invention via raw log 2 transformed proteome-wide multi-isotype autoantibody immunoprofiling of cohort of patients with ulcerative colitis in comparison with a cohort of healthy patients. Depicted are volcano plots of (X-axis—fold autoantibody signal enrichment in ulcerative colitis in controls; Y-axis—absolute value of the Log 10_P-value by T-test); the protein TOLLIP (Toll Interacting Protein) is the most statistically significant IgA autoantibody differentiator for ulcerative colitis versus healthy controls and autoantibodies to TOLLIP could be the mechanistic cause of ulcerative colitis.

FIG. 33 depicts the utility of this invention utilizing raw log 2 transformed proteome-wide multi-isotype autoantibody immunoprofiling of cohort of patients with ulcerative colitis in comparison with a cohort of healthy patients; the protein TOLLIP (Toll Interacting Protein) is the most statistically significant IgA autoantibody differentiator for ulcerative colitis in this study; this signal is very biased for IgA response versus the other isotypes IgG1, IgG2, IgG3, IgG4, and IgM—more than for any other human protein in the study. TOLLIP is a secreted protein that regulates the activity of cytotoxic neutrophils in the colon in the presence of bacterial components via TLR receptors. Others have reported that low TOLLIP activity could lead to ulcerative colitis. The presence of anti-TOLLIP IgA autoantibodies secreted in the colonic mucosa could block this natural tonic function, creating a permanent mechanistic cause for the chronic condition ulcerative colitis that was previously unknown and was identified by the utility of methods presented herein.

FIG. 34 depicts survival data for cancer patients overexpressing TOLLIP protein having poorer overall survival in The Cancer Genome Atlas (TCGA) metastatic melanoma cohort; these data support TOLLIP being a druggable immune checkpoint—such as might be blocked naturally by natural colonic IgA autoantibodies to cause ulcerative colitis.

FIG. 35 depicts survival data for cancer patients overexpressing TOLLIP protein having poorer overall survival in The Cancer Genome Atlas (TCGA) glioma brain cancer cohort. these data support TOLLIP being a druggable immune checkpoint such as might be blocked naturally by natural colonic IgA autoantibodies to cause ulcerative colitis.

### DETAILED DESCRIPTION OF THE INVENTION

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

Some inventive embodiments herein contemplate numerical ranges. When ranges are present, the ranges include the range endpoints. Additionally, every sub range and value within the rage is present as if explicitly written out. The term “about” or “approximately” can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. The term “about” has the meaning as commonly understood by one of ordinary skill in the art. In some embodiments, the term “about” refers to ±10%. In some embodiments, the term “about” refers to ±5%.

The terms “attach”, “bind”, “couple”, and “link” are used interchangeably and refer to covalent interactions (e.g., by chemically coupling), or non-covalent interactions (e.g., ionic interactions, hydrophobic interactions, hydrogen bonds, hybridization, etc.). The terms “specific”, “specifically”, or specificity” refer to the preferential recognition, contact, and formation of a stable complex between a first molecule and a second molecule compared to that of the first molecule with any one of a plurality of other molecules (e.g., substantially less to no recognition, contact, or formation of a stable complex between the first molecule and any one of the plurality of other molecules). For example, two molecules may be specifically attached, specifically bound, specifically coupled, or specifically linked. For example, specific hybridization between a first polynucleotide and a second polynucleotide can refer to the binding, duplexing, or hybridizing of the first polynucleotide preferentially to a particular nucleotide sequence of the second polynucleotide under stringent conditions. Sufficient number complementary base pairs in a polynucleotide sequence may be required to specifically hybridize with a target nucleic acid sequence. A high degree of complementarity may be needed for specificity and sensitivity involving hybridization, although it need not be 100%.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal and a non-mammal. A mammal can be any member of the Mammalian class, including but not limited to a human; a non-human primates such as a chimpanzee, an ape or other monkey species; a farm animal such as cattle, a horse, a sheep, a goat, a swine; a domestic animal such as a rabbit, a dog (or a canine), and a cat (or a feline); or a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, the human can be male or female. In some instances, the human can be an adult. In some instances, the human can be a child. In some instances, the human can be age 0-17 years old. In some instances, the human can be age 18-130 years old. In some instances, the subject is diagnosed with, or is suspected of having, a condition or disease such as cancer. In instances, the subject is diagnosed with, or is suspected of having lung cancer.

The term “non-diseased subject” as used herein can encompass a healthy individual. A healthy individual can be an individual without a particular disease. For example, a healthy individual can be an individual without a cancer such as lung cancer.

The terms “treat,” “treating”, “treatment,” “ameliorate” or “ameliorating” and other grammatical equivalents as used herein, can include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms can further include achieving a therapeutic benefit and/or a prophylactic benefit. Therapeutic benefit can mean eradication or amelioration of the underlying disease being treated. Also, a therapeutic benefit can be achieved with the eradication or amelioration of one or more of the physiological symptoms associated with the underlying disease such that an improvement can be observed in the patient, notwithstanding that, in some embodiments, the patient can still be afflicted with the underlying disease.

The term “isolated” as used herein can refer to the removal of a protein, or nucleic acid from its endogenous or natural environment. For example, a protein may be isolated by removal and separation from an organism, bodily fluid, or fermentation medium.

The term “probe” as used herein can refer to any molecular moiety that can be identified to detect the presence of the probe. A probe may be used to detect a target molecule or moiety to which the probe is associated. The association of a probe to a target may be covalent or non-covalent. In some embodiments, a probe may comprise any detectable moiety, such as a fluorescent dye, a phosphor, a radiolabel, or a chromophore. The term “fluorescent dye” as used herein refers to molecular moiety comprising a fluorophore that can be used as a probe. In some aspects, a chromophore may absorb, reflect, or emit light at ultraviolet or visible wavelengths.

The terms “early stage” and “late stage” as used herein can refer to the degree of progression of a disease. For example, an early stage lung cancer can be a stage I lung cancer or a stage II lung cancer. In some instances, an early stage lung cancer can be confined to the lung tissue of a subject. In some instances, an early stage lung cancer can be a limited stage small cell lung cancer. In some instances, a late stage lung cancer can be a stage III or stage IV lung cancer. In some instances, a late stage lung cancer can have spread from the lung tissue to another organ of a subject. In some instances, a late stage lung cancer can be an extensive stage small cell lung cancer.

The terms “homologous,” “homology,” or “percent homology” as used herein refer to the degree of sequence similarity between an amino acid or nucleotide sequence and a reference sequence. As used herein, the term “homology” can be used interchangeably with the term “identity.” In some instances, percent sequence homology can be determined using the formula described by Karlin and Altschul. Such a formula is incorporated into the basic local alignment search tool (BLAST). Percent homology of sequences can be determined using the most recent version of BLAST, as of the filing date of this application. In some instances, percent homology of sequences can be determined using Smith-Waterman homology search algorithm.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues. Most sequence comparison method over longer sequences are designed to produce optimal alignments that take into consideration possible insertions and deletions without penalizing unduly the overall homology score. This is achieved by inserting “gaps” in the sequence alignment to try to maximize local homology. these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimized alignments with fewer gaps. Most alignment programs allow the gap penalties to be modified. Typically, the default values are used when using such software for sequence comparisons. Calculation of maximum % homology therefore firstly requires the production of an optimal alignment, taking into consideration gap penalties. Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. In some instances, an alignment is determined by the Smith-Waterman homology search algorithm using an affine gap search with a gap open penalty of 12 a gap extension penalty of 2, and a blocks substitution matrix (BLOSUM) of 62.

In some embodiments, the sample can be an antibody, nucleic acid (DNA or RNA) or antigen containing sample. In some embodiments, an antibody or antigen containing sample can be a biological fluid. In some embodiments, the sample can be a biological fluid. A biological fluid prepared for analysis in the process described herein include or can include a host of potential biomarkers including markers expressed on cells (non-adherent cells, including T-cells or other immune effector cells), microorganisms, proteins, peptides, lipids, polysaccharides, small molecules, organic molecules, inorganic molecules, biological molecules and including any detectable or reactable moiety in such complex milieu. In some embodiments, such antibodies and, in particular, can be antibodies generated as a result of a disease or condition. In some embodiments, body fluids such as serum, plasma, saliva, urine or other fluids or samples derived from a subject or animal or organism can be the source of such biomarkers. In some embodiments, the sample can be blood, serum, saliva or CSF. In some embodiments, the sample can be for example, sputum, bone marrow, synovial fluid, aqueous humor, amniotic fluid, cerumen, breast milk, broncheoalveolar lavage fluid, semen, prostatic fluid, Cowper's fluid, pre-ejaculatory fluid, female ejaculate, sweat, tears, cyst fluid, pleural fluid, peritoneal fluid, pericardial fluid, lymph, chyme, chyle, bile, interstitial fluid, menses, pus, sebum, vaginal secretion, mucosal secretion, stool water, pancreatic juice, lavage fluid from sinus cavities, bronchopulmonary aspirate, blastocyl cavity fluid, or umbilical cord blood. In some embodiments, a sample can be a cell-free sample.

As used herein, the term “cell-free” can refer to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample. A cell-free nucleic acid sequence may comprise a complementary DNA (cDNA). In some cases, one or more cDNAs may form a cDNA library.

In some embodiments, a sample can be from a subject afflicted with a disease or condition disclosed herein. In some embodiments, a subject from which a sample is obtained can have a disease or condition. In some embodiments, the disease or condition can be a cancer, depression, obesity, cardiovascular disease, diabetes, organ failure, solid organ transplant, bone marrow transplant, neurodegeneration, muscle degeneration, allergies, food intolerances, neurodegenerative disease autism, epilepsy, and schizophrenia, healthy ageing, unhealthy ageing, SARS-CoV-2 (COVID-19), HIV or a bacterial infection.

A protein or antigen can be coupled to a solid support (e.g., an array or bead). In some instances, a protein can be non-covalently coupled to a solid support. For example, a non-covalent interaction can be an ionic interaction or a van der Waals interaction. In some instances, a protein can be covalently coupled to a solid support. In some instances, a protein can be reversibly coupled to a solid support. In some instances, a protein is irreversibly coupled to a solid support.

A surface of a solid support can be coated with a functional group and a protein can be attached to the solid support through the functional group. For example, a solid support can be coated with a first functional group and a protein comprising a second functional group can be attached to the solid support by reacting the first functional group with the second functional group. For example, a surface of a solid support can be coated with streptavidin and a biotinylated protein can be attached thereto. Exemplary couplings of a protein include streptavidin- or avidin- to biotin interactions; hydrophobic interactions; magnetic interactions; polar interactions, (e.g., associations between two polar surfaces); formation of a covalent bond (e.g., an amide bond, disulfide bond, thioether bond, or via crosslinking agents; and via an acid-labile linker.

In some embodiments, the surface of a solid support can be coated with an affinity ligand. In some embodiments, an affinity ligand can include, but is not limited to an antigen, an antibody, an antibody fragment, glutathione, calmodulin, biotin, streptavidin, streptactin, amylose, an anion-exchange resin such as Mono-Q, FlAsH and ReAsH biarsenical compounds, pilin-C protein, SpyCatcher protein or a metal chelate. In some instances, the metal chelate can include but is not limited to nickel, cobalt, zinc, mercury, copper, or iron chelate. In some embodiments, the solid support can be coated entirely. In some embodiments, the solid support can be coated partially. In some embodiments, proteins can comprise an affinity tag and solid support can comprise an affinity ligand, thus coupling the proteins to the solid support by reacting the affinity tag with the affinity ligand.

In some instances, a protein can be coupled to a solid surface through a linker. For example, a first functional group of a linker attached to a solid surface can be coupled to a protein, thereby coupling the protein to the solid surface. For example, a first functional group of a linker can be coupled to a protein and a second functional group of the linker can be coupled to a solid support, thereby coupling the protein to the solid surface. Proteins can be coupled to a solid surface through a linker. In some instances, a linker comprising a first and a second functional group can be attached to the solid support via the second functional group after the first functional group is coupled to the protein. In some instances, a linker comprising a first and a second functional group can be attached to the solid support via the second functional group before the first functional group is coupled to the protein.

In some instances, a protein can be coupled to a solid surface via an antibody. For example, an antibody linker can be attached to a solid surface and a protein to which the antibody specifically binds can be linked to the solid support by binding to the antibody linker. In some instances, the coupling is photocleavable. In some instances, proteins can comprise a tag that is directly coupled to a solid surface. For example, protein can comprise a fusion tag that is directly conjugated to the solid surface. For example, a protein can comprise a GST-tag, His-tag, FLAG-tag, or other similar tags and the tag can be directly coupled to the solid surface instead of the protein itself.

There are many known methods for covalently immobilizing polypeptides and antibodies onto a solid support. For example, the Michael addition can be used to link thiol-containing compounds to maleimide-derivatized glass slides to form a microarray of small molecules. Non-covalent coupling may be by any suitable secondary interaction, including but not limited to hydrophobic bonding, hydrogen bonding, Van der Waals interactions, ionic bonding, etc.

Amine chemistry can be used to couple or immobilize proteins to a solid surface. For example, a covalent amide bond can be formed between a protein and a solid support. For example, a covalent amide bond can be formed by reacting a carboxyl-functionalized protein with an amino-functionalized solid support. For example, a covalent amide bond can be formed by reacting an amide-functionalized protein with a carboxyl-functionalized solid support. Amine-terminated protein may be immobilized using amine/cyanuric chloride coupling; amide bonding through reactions with N-hydroxysuccinimide (NHS)-ester-, carboxylic acid-, carbonate-, anhydride- or acyl group-functionalized surfaces; amidine formation through reaction with imidoester-functionalized surfaces; sulphonamide formation through reactions with sulfonyl halide-functionalized surfaces; aniline formation through reactions with surface presenting aryl groups; imine formation through reactions with aldehyde-functionalized surfaces; amino ketone formation through Mannich reactions with aldehyde-functionalized surfaces; guanidine formation through reactions with carbodiimide-functionalized surfaces; urea formation through reactions with isocyanate-functionalized surfaces; thiourea formation through reactions with isothiocyanate-functionalized surfaces, or; amino alcohol formation through reactions with epoxide-functionalized surfaces. Hydrazine- or oxyamine-terminated binding agents may be immobilized in the same way.

Thiol groups can be used to couple or immobilize protein to a solid surface. For example, protein having or functionalized with thiol groups with may be immobilized on surfaces presenting, e.g., maleimide, aryl- or carbon-carbon double-bond-containing groups through formation of stable carbon-sulfur bonds, or through interactions with aziridine-functionalized surfaces. Disulfide exchange reactions with thiol-functionalized surfaces may also be used. Protein having or functionalized with thiol groups may be immobilized on gold surfaces through semi-covalent interactions between gold and sulphur groups. Carboxylic acid-functionalized surfaces may also be used to immobilize protein functionalized with carbodiimide and diazoalkane groups. Solid surfaces presenting hydroxyl groups may be used to immobilize isocyanate- and epoxide-functionalized proteins.

Functionalized protein may also be immobilized through cycloaddition reactions between functional groups having a conjugated diene and groups having a substituted alkene through Diels-Alder chemistry, or using “click” chemistry, through reactions between nitrile and azine groups. In any of the above described covalent couplings, the protein-surface orientation of functional groups may be reversed. An alternative means of covalent attachment not utilizing a derivatized binding agent utilizes array surfaces having photoreactive groups such as benzophenone, diazo, diazirine, phthalamido and arylazide groups.

Non-covalent immobilization may involve electrostatic interactions between proteins and surfaces modified to contain positively- or negatively-charged groups, such as amine or carboxy groups, respectively. Proteins may be non-covalently immobilized in a defined orientation, for example, using fluorophilic, biotin-streptavidin, histidine-Ni, histidine-Co, and complementary single-stranded DNA interactions between tagged proteins and binding partner-coated surfaces, in either orientation.

Appropriate agents for coupling of proteins to a solid surface include a variety of agents that are capable of reacting with a functional group present on a surface of the protein and with a functional group present on the solid surface. Reagents capable of such reactivity include homo- and hetero-bifunctional reagents. A bifunctional cross-linking agent can comprise N-succinimidyl(4-iodoacetyl) aminobenzoate (SIAB), dimaleimide, dithio-bis-nitrobenzoic acid (DTNB), N-succinimidyl-S-acetyl-thioacetate (SATA), N-succinimidyl-3-(2-pyridyldithio) propionate (SPDP), succinimidyl 4-(N-maleimidomethyl) cyclohexane-1-carboxylate (SMCC) and 6-hydrazinonicotimide (HYNIC). Any suitable nucleophile reactive group can be used including —NR1-NH2 (hydrazide), —NR1(C═O)NR2NH2 (semicarbazide), —NR1(C═S)NR2NH2 (thiosemicarbazide), (C═O)NR1NH2 (carbonylhydrazide), —(C═S)NR1NH2 (thiocarbonylhydrazide), —(S02)NR1NH2 (sulfonylhydrazide), —NR1NR2(C)NR3NH2 (carbazide), —NR1NR2(C═S)NR3NH2 (thiocarbazide), and —O—NH2 (hydroxylamine), where each R1, R2, and R3 is independently H, or alkyl having 1-6 carbons. A nucleophilic moiety can include any suitable nucleophile, e.g., hydrazide, hydroxylamine, semicarbazide, or carbonylhydrazide.

In addition to those described above, other covalent and non-covalent means of attachment may be employed and are well known to those skilled in the art. A protein may be deposited onto a substrate or support by any suitable technique. For example, a protein may be deposited as a monolayer (e.g., a self-assembled monolayer), a continuous layer or as a discontinuous (e.g., patterned) layer. A protein may be deposited or coupled to a support or substrate by modification of the substrate or support by chemical reaction (See, e.g., U.S. Pat. No. 6,444,254), reactive plasma etching, corona discharge treatment, a plasma deposition process, spin coating, dip coating, spray painting, deposition, printing, stamping, diffusion, adsorption/absorption, covalent cross-linking, or combinations thereof. The proteins may be directly spotted onto a surface (e.g., a planar glass surface). In some instances, when necessary or beneficial to keep proteins (e.g., Abs) in a wet environment during the printing process, glycerol (30-40%) may be employed, and/or spotting can be carried out in a humidity-controlled environment.

An autoantibody can be an antibody produced by a subject's immune system that is directed against one or more of a subject's own molecule. A subject's own molecule can comprise a peptide, an amino acid, a protein, a saccharide, a polysaccharide, a nucleic acid, a lipid, complexes of any of these, or any combination thereof. An autoantibody should be understood to be an antibody, and an antibody may be an autoantibody. In some embodiments, an autoantibody is directed to one or more of a subject's own proteins. In some embodiments, an autoantibody binds to one or more of a subject's proteins. In some embodiments, an autoantibody binds to a protein. An antibody, including an autoantibody, can specifically bind to a particular portion of a protein, including a protein. The portion of a protein to which is an antibody binds may be called an antigenic sequence. The portion of a protein to which is an antibody binds may be also be referred to as an epitope or an antigenic determinant. An antigenic sequence or epitope may comprise a continuous sequence of amino acids, or it may comprise discontinuous sections of the target protein's amino acid sequence. In some embodiments, an antigenic sequence or epitope of a protein may comprise 1, 2, 3, 4, 5, 6, 7, 8, 9, 10, 11, 12, 13, 14, 15, 16, 17, 18, 19, or 20 amino acids. An antibody can have strong binding affinity for an antigenic sequence or epitope, as measured by its dissociation constant. In some embodiments, an antibody can have dissociation constant of less than about 10⁻⁵M, 10⁻⁶M, 10⁻⁷M, 10⁻⁸M, 10⁻⁹M, 10⁻¹⁰M, 10⁻¹¹M, 10¹²M, 10⁻¹³M, or 10⁻¹⁴M.

In some embodiments, an amino acid of a protein disclosed herein can be modified. A modification described herein can include acetylation, acylation, adenylylation, amidation, arginylation, biotinylation, carbamylation, carbonylation, carboxylation, citrullination, eliminylation, farnesylation, formylation, glycation, glycosylation, glypiation, hydroxylation, imination, isoprenylation, lipidation, lipoylation, malonylation, methylation, myristoylation, Neddylation, nitrosylation, oxidation, palmitoylation, pegylation, phophopantetheinylation, phosphorylation, polyglutamylation, prenylation, Pupylation, succinylation, sulfation, sumoylation, ubiquitylation, and/or any combination thereof.

In some embodiments, a residue of a protein can be modified. In some embodiments, residue modification can comprise the absence of a residue or the absence of a fragment of a residue. In one example, a methyl group can be absent or has been removed. In some embodiments, the modified residue can comprise de-acetylation, de-acylation, de-adenylylation, de-amidation, de-arginylation, de-biotinylation, de-carbamylation, de-carbonylation, de-carboxylation, de-citrullination, de-eliminylation, de-farnesylation, de-formylation, de-glycation, de-glycosylation, de-glypiation, de-hydroxylation, de-imination, de-isoprenylation, de-lipidation, de-lipoylation, de-malonylation, de-methylation, de-myristoylation, de-Neddylation, de-nitrosylation, de-oxidation, de-palmitoylation, de-pegylation, de-phophopantetheinylation, de-phosphorylation, de-polyglutamylation, de-prenylation, de-Pupylation, de-succinylation, de-sulfation, de-sumoylation, de-ubiquitylation, and/or any combination thereof.

An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can be comprised of three domains, C_{H1}, C_{H2} and C_{H3}. Each light chain can be comprised of a light chain variable region (V_{L}) and a light chain constant region. The light chain constant region can be comprised of one domain, C_{L}. The V_{H} and V_{L} regions can be further subdivided into regions of hypervariability, termed complementary determining regions (CDR), interspersed with regions that are more conserved, termed framework regions (FR). Each VH and V_{L} can be composed of three CDRs and four FRs arranged from amino-terminus to carboxy-terminus in the following order: FR₁, CDR₁, FR₂, CDR₂, FR₃, CDR₃, and FR₄. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., lgG₁, lgG₂, lgG₃, lgG₄, lgA₁ and lgA₂), subclass or modified version thereof. Antibodies may include a complete immunoglobulins or fragments thereof. An antibody fragment can refer to one or more fragments of an antibody that retain the ability to specifically bind to a protein. In addition, aggregates, polymers, and conjugates of immunoglobulins or their fragments can be used where appropriate so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal. In some aspects, an antibody can be produced as a result of a disease, disorder, or a condition.

The methods provided herein comprise forming and or detecting complexes. A complex can refer to an association between at least two moieties (e.g. chemical or biochemical) that have an affinity for one another. The methods provided herein comprise forming a complex between a protein and an antibody, such as an autoantibody. In some embodiments, the methods comprise forming a complex between a protein and a single autoantibody. In some embodiments, a protein can comprise a peptide. In some embodiments, a peptide can comprise more than one protein. In some embodiments, a peptide can comprise multiple proteins. In some embodiments, the method can comprise forming a complex between a protein and a complex of two or more antibodies. In some embodiments, a method can comprise forming a complex between a protein and a complex of two or more antibodies. In some embodiments, a method can comprise forming a complex between two or more proteins and a complex of two or more antibodies. In some embodiments, a method can comprise forming a complex between a first complex comprising a protein and another moiety (e.g., a polypeptide, polynucleotide, or small molecule) and an antibody. In some embodiments, a method can comprise forming a complex between a first complex comprising a protein and another moiety (e.g., a polypeptide, polynucleotide, or small molecule) and a second complex comprising two or more antibodies. For example, complexes can be formed between a protein coupled to a solid support, and an antibody to a protein.

Detection methods for detecting protein-antibody complexes can include photometric and non-photometric means. In some embodiments, a detection can comprise a method to detect and measure absorbance, fluorescence, phosphorescence, refractive index, polarization or light scattering. These include direct and/or indirect means to measure such parameters. Methods involving fluorescence include fluorescent tagging in immunological methods such as ELISA or sandwich assay. Methods involving refractive index include surface Plasmon resonance (SPR), grating coupled methods (e.g. sensors uniform grating couplers, wavelength-interrogated optical sensors (WIOS) and chirped grating couplers), resonant minor and interferometric techniques. Methods involving polarization include ellipsometry. Light scattering methods may also be used. Other means for tagging and/or separating and/or detecting can also include magnetic means. Magnetic resonance imaging, gas phase ion spectrometry, MRI may all be used.

Non-photometric methods of detection include, without limitation, magnetic resonance imaging, gas phase ion spectrometry, atomic force microscopy and multipolar coupled resonance spectroscopy. Magnetic resonance imaging (MRI) is based on the principles of nuclear magnetic resonance (NMR), a spectroscopic technique used by scientists to obtain microscopic chemical and physical information about molecules. Gas phase ion spectrometers include mass spectrometers, ion mobility spectrometers and total ion current measuring devices.

Mass spectrometers measure a parameter which can be translated into mass-to-charge ratios of ions. Generally, ions of interest bear a single charge, and mass-to-charge ratios are often simply referred to as mass. Mass spectrometers include an inlet system, an ionization source, an ion optic assembly, a mass analyzer, and a detector. Several different ionization sources have been used for desorbing and ionizing analytes from the surface of a support or biochip in a mass spectrometer. Such methodologies include laser desorption/ionization (MALDI, SELDI), fast atom bombardment, plasma desorption, and secondary ion mass spectrometers. In such mass spectrometers the inlet system can comprise a support interface capable of engaging the support and positioning it in interrogatable relationship with the ionization source and concurrently in communication with the mass spectrometer, e.g., the ion optic assembly, the mass analyzer and the detector. Solid supports for use in bioassays that have a generally planar surface for the capture of targets and adapted for facile use as supports with detection instruments are generally referred to as biochips.

Analysis of the data generated typically involves quantification of a signal due to the detected antibody versus a control or reference. The data can be analyzed by any suitable means. Computers and computer programs may be utilized to generate and analyze the data. Beads and/or other supports may be computer coded or coded for identification purposes. Data analysis includes analysis of signal strength under the particular conditions of the assay or detection method. Proteins, antibodies, reference moieties and/or secondary detection moieties may be labeled or radio-labeled or tagged with a detectable moiety. One of ordinary skill in the art can also determine, pursuant to the methods described herein, the presence of false positives or other hits that are or may be found in control samples to account for and/or remove such hits and one of ordinary skill in the art, pursuant to the methods described herein, can continue the process of determining or finding disease associated biomarkers in subject samples having any disease or condition. The detection of such hits, in all cases, can be accomplished by sequencing.

The term “sequencing” as used herein, may comprise high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore DNA sequencing, shot gun sequencing, RNA sequencing, Enigma sequencing, or any combination thereof.

In some embodiments, detecting a binding of a moiety can comprise sequencing the moiety. In some embodiment, sequencing can be performed by nanopore sequencing. For example, Oxford nanopore sequencing. Nanopores may be used to sequence, a sample, a small portion (such as one full gene or a portion of one gene), a substantial portion (such as multiple genes or multiple chromosomes), or the entire genomic sequence of an individual. Nanopore sequencing technology may be commercially available or under development from Sequenom (San Diego, Calif.), Illumina (San Diego, Calif.), Oxford Nanopore Technologies LTD (Kidlington, United Kingdom), and Agilent Laboratories (Santa Clara, Calif.). Nanopore sequencing methods and apparatus are have been described in the art and for example are provided in U.S. Pat. No. 5,795,782, herein incorporated by reference in its entirety.

Nanopore sequencing can use electrophoresis to transport a sample through a pore. A nanopore system may contain an electrolytic solution such that when a constant electric field is applied, an electric current can be observed in the system. The magnitude of the electric current density across a nanopore surface may depend on the nanopore's dimensions and the composition of the sample that is occupying the nanopore. During nanopore sequencing, when a sample approaches and or goes through the nanopore, the samples cause characteristic changes in electric current density across nanopore surfaces, these characteristic changes in the electric current enables identification of the sample. Nanopores used herein may be solid-state nanopores, protein nanopores, or hybrid nanopores comprising protein nanopores or organic nanotubes such as carbon or graphene nanotubes, configured in a solid-state membrane, or like framework. In some embodiments, nanopore sequencing can be biological, a solid state nanopore or a hybrid biological/solid state nanopore.

In some instances, a biological nanopore can comprise transmembrane proteins that may be embedded in lipid membranes. In some embodiments, a nanopore described herein may comprise alpha hemolysin. In some embodiments, a nanopore described herein may comprise Mycobacterium smegmatis porin.

Solid state nanopores do not incorporate proteins into their systems. Instead, solid state nanopore technology uses various metal or metal alloy substrates with nanometer sized pores that allow samples to pass through. Solid state nanopores may be fabricated in a variety of materials including but not limited to, silicon nitride (Si3N4), silicon dioxide (Si02), and the like. In some instances, nanopore sequencing may comprise use of tunneling current, wherein a measurement of electron tunneling through bases as sample (ssDNA) translocates through the nanopore is obtained. In some embodiments, a nanopore system can have solid state pores with single walled carbon nanotubes across the diameter of the pore. In some embodiments, nanoelectrodes may be used on a nanopore system described herein. In some embodiments, fluorescence can be used with nanopores, for example solid state nanopores and fluorescence. For example, in such a system a fluorescence sequencing method can convert each base of a sample into a characteristic representation of multiple nucleotides which bind to a fluorescent probe strand-forming dsDNA (where a sample comprises DNA). Where a two color system is used, each base is identified by two separate fluorescence, and will therefore be converted into two specific sequences. Probes may consist of a fluorophore and quencher at the start and end of each sequence, respectively. Each fluorophore may be extinguished by the quencher at the end of the preceding sequence. When the dsDNA is translocating through a solid state nanopore, the probe strand may be stripped off, and the upstream fluorophore will fluoresce.

In some embodiments, a 1-100 nm channel or aperture may be formed through a solid substrate, usually a planar substrate, such as a membrane, through which an analyte, such as single stranded DNA, is induced to translocate. In other embodiments, a 2-50 nm channel or aperture is formed through a substrate; and in still other embodiments, a 2-30 nm, or a 2-20 nm, or a 3-30 nm, or a 3-20 nm, or a 3-10 nm channel or aperture if formed through a substrate.

In some embodiments, nanopores used in connection with the methods and devices of the invention are provided in the form of arrays, such as an array of clusters of nanopores, which may be disposed regularly on a planar surface. In some embodiments, clusters are each in a separate resolution limited area so that optical signals from nanopores of different clusters are distinguishable by the optical detection system employed, but optical signals from nanopores within the same cluster cannot necessarily be assigned to a specific nanopore within such cluster by the optical detection system employed means for detecting the binding of a protein to an antibody.

Binding assays can also be useful, e.g., for identifying disease related antibodies that interact with the proteins described herein. For example, antibodies or other molecules that bind proteins of the invention can be identified in binding assays. Binding assays can involve, but are not limited to, use of isolated polypeptides, crude extracts, or cell-based assays. In some embodiments the assays described herein can be used to a) identify subjects whose have a first disease or a second disease; (b) assess the impact of a disease therapy; and (c) monitor disease progression.

Binding assays can involve contacting a protein with a sample comprising an antibody and allowing sufficient time for the molecule and test agents to form a binding complex. Any binding complexes formed can be detected using any of a number of established analytical techniques. Binding assays include, but are not limited to, methods that measure co-precipitation or co-migration on non-denaturing SDS-polyacrylamide gels, co-migration on Western blots, enzyme linked immunosorbent assay (ELISA), radioimmunoassay (RIA), immunoradiometric assay, fluoroimmuno assay, chemiluminescent assay, bioluminescent assay, FACS, FRET. The steps of various useful immunodetection methods have been described in the scientific literature. Other binding assays involve the use of mass spectrometry or NMR techniques to identify target analyte bound the antibody or displacement of labeled substrates. The antibodies used in these assays can be naturally expressed, cloned or synthesized. In addition, mammalian or yeast two-hybrid approaches can be used to identify polypeptides or other molecules that interact or bind to the polypeptide when expressed together in a host cell. U.S. Pat. Nos. 3,817,837, 3,850,752, 3,939,350, 3,996,345, 4,277,437, 4,275,149 and 4,366,241 are hereby incorporated by reference in its entirety.

Immunoassays, in their most simple and direct sense, are binding assays. Certain immunoassays finding particular use in the present invention are various types of enzyme linked immunosorbent assays (ELISAs) and radioimmuno assays (RIA) known in the art. In one exemplary ELISA, the protein of the invention are immobilized onto a selected surface, such as a well in a polystyrene microtiter plate. Then, a test composition suspected of containing the antibody is added to the wells. After binding and washing to remove non-specifically bound complexes, the bound antibody may be detected. Detection may be achieved by the addition of another ligand linked to a detectable label. This type of assay is analogous to a simple “sandwich ELISA” except that binding of the labeled agent is direct at the Fab portion of the bound antibody. Detection may also be achieved by the addition of a labeled antibody that binds any bound antibody, e.g., that recognizes the Fc portion of the bound antibody. Optionally, this antibody is not labeled, and is followed by the addition of a second antibody that has binding affinity for the first antibody, with the second antibody being linked to a detectable label.

In another exemplary ELISA, the samples suspected of containing the antibodies are immobilized onto a well surface and then contacted with labeled proteins of the present invention. After binding and washing to remove non-specifically bound immune complexes, the bound labeled ligands are detected. Alternatively, the ligands are not labeled and can be detected against an artificial antibody (non-sample) that is selected for specific binding the protein of choice, this second would be linked to a detectable label, thereby permitting detection.

Irrespective of the format employed, ELISAs have certain features in common, such as coating, incubating and binding, washing to remove non-specifically bound species, and detecting the bound immune complexes. These are described below.

In coating a plate with either protein or antibody, one can generally incubate the wells of the plate with a solution of the protein or antibody, either overnight or for a specified period of hours. In certain aspects, the plate can be blocked using a bacterial lysate, such as an E. coli lysate (See Example 1). The wells of the plate can then be washed to remove incompletely adsorbed material. Any remaining available surfaces of the wells can then be coated with a non-specific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a target analyte or antibody to the well, coating with a non-reactive material to reduce background, and washing to remove unbound material, the immobilizing surface can be contacted with a biological sample or protein to be tested under conditions effective to allow immune complex formation. Detection of the immune complex then requires a labeled secondary binding ligand or antibody, or a secondary binding ligand or antibody in conjunction with a labeled tertiary antibody (with specificity either for the Fc region of the antibody or the ligand).

Under conditions effective to allow immune complex (antigen/antibody) formation means that the conditions can include diluting the antigens and/or antibodies with solutions such as BSA, bovine gamma globulin (BGG) or phosphate buffered saline (PBS)/Tween. These added agents can assist in the reduction of nonspecific background.

The suitable conditions can also mean that the incubation is at a temperature or for a period of time sufficient to allow effective binding. Incubation steps can be from about 1 to 2 to 4 to 6 to 24 to about 48 hours or so, at temperatures on the order of about 20° C. to about 37° C. In some embodiments, about 21° C., 22° C., 23° C., 24° C., 25° C., 26° C., 27° C., 28° C., 29° C., 30° C., 31° C., 32, ° C., 33° C., 34° C., 35° C., 36° C., or about 37° C. or may be overnight at about 2° C., 3° C., 4° C., 5° C., 6° C., 7° C. or so.

Following incubation steps in an ELISA, the contacted surface can be washed so as to remove non-complexed material. In some embodiments, the washing procedure includes washing with a solution such as PBS/Tween, or borate buffer. Following the formation of specific immune complexes between the test sample and the originally bound material, and subsequent washing, the occurrence of even minute amounts of immune complexes may be determined.

Detection may utilize an enzyme that can generate color development upon incubating with an appropriate chromogenic substrate. Thus, for example, one can desire to contact or incubate the first and second immune complex with a urease, glucose oxidase, alkaline phosphatase or hydrogen peroxidase-conjugated antibody or ligand for a period of time and under conditions that favor the development of further immune complex formation (e.g., incubation for about 1, 2, 3, 4, 5, 6, 7, 8, 9, or about 10 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label.

Quantification can be achieved by measuring the degree of color generated, e.g., using a visible spectra spectrophotometer.

FRET is a phenomenon in which the excited-state energy in one molecule (called the donor) is transferred to another molecule by a radiationless coupling. This mechanism was first correctly described by Förster, and differs from other types of energy transfer, such as electron sharing (Dexter) or trivial transfer (emission of a photon from the donor and reabsorption by the acceptor). The Dexter mechanism requires the two molecules to be in physical contact, while trivial transfer is a very low probability. In contrast, the Förster mechanism exhibits a high probability when the two molecules are within the Förster radius, which is defined for any given pair of fluorophores.

The overall FRET efficiency depends on the Förster radius, and is determined by several factors and is directly related to the amount of overlap between the absorption spectra of the acceptor molecule and the emission spectra of the donor molecule. The amount of FRET also depends on the alignment of the donor and acceptor molecules, although most biological systems are not rigidly aligned. The FRET efficiency is also affected by the ability of the acceptor molecule to absorb light, as indicated by its molar extinction coefficient, and the overall stability of the excited state of the donor molecule, as indicated by the probability that absorption will lead to fluorescence (quantum yield) and the lifetime of the excited state.

FRET between two different fluorophores can be assayed by several methods: looking at the change in color of the fluorescence, measuring the fluorescence lifetime of the donor, examining the changes upon photo bleaching either the donor or acceptor, or by measuring the fluorescence polarization of the acceptor. Regardless of the approach, most of these assays share common features of the instrumentation.

The types of microscopes used to measure FRET can be suitably selected depending on the purpose. In some embodiments, where frequent observations are necessary for monitoring a time course of the changing, conventional incident-light fluorescent microscope can be used. In some embodiments, where resolution is to be increased as in the case where detailed intercellular localization is to be monitored, confocal laser microscope can be used. As a microscope system, an inverted microscope can be used for most live cell measurements in view of keeping the physiological state of cell and preventing contamination. When an upright microscope is used, a water immersion lens can be used in the case of using lens of high power.

The filter set can be suitably selected depending on the fluorescent wave length of the fluorescent protein. For the observation of GFP, a filter with excitation light of about 470-490 nm and fluorescent light of about 500-520 nm can be used. For the observation of YFP, a filter with excitation light of about 490-510 nm and fluorescent light of about 520-550 nm can be used. For the observation of CFP, it is preferred to use a filter with excitation light of about 425 nm and fluorescent light of about 460-500 nm. Moreover, when time course observation is carried out in living cells by using a fluorescent microscope, the cells can be photographed in a short period, and therefore a high sensitivity cooled CCD camera can be used. By using a cooled CCD camera, thermal noise can be decreased by cooling CCD, and weak fluorescent image can be clearly acquired by exposure of short period. Confocal microscopes can also be used for live cell imaging, as long as care is taken to minimize the exposure times.

In a similar manner, any ligand may be screened on the beads or supports using the processes described herein. These ligands include, in addition to peptoids or peptides, nucleic acid oligomers, polysaccharides, small molecules and/or any combination thereof which can be built into libraries and, under the conditions recited herein, used to screen biological fluid.

In some embodiments, detecting may comprise radio immunoassay (“RIA”), fluorescence immunoassay (“FIA”), enzyme-linked immunosorbent assay (“ELISA”), Western blot, flow cytometry, Forster resonance energy transfer (“FRET”), or surface plasmon resonance.

The methods, kits, and compositions described herein can be used for numerous applications, including identification of binding partners, determination of affinities of antibodies to proteins, determination of specificities of antibodies to proteins, quantification of proteins in a sample, quantification of antibodies in a sample, quantification of binding events, identification of a disease, identification of biomarkers of a disease or condition, drug discovery, molecular biology, immunology, toxicology, antibody isotype mapping or profiling, detecting responsiveness to treatment and response to disease. Arrays can be used for large scale binding assays in numerous diagnostic and screening applications. These methods of use include, but are not limited to, high-content, high-throughput assays for screening for antibodies that interact with proteins. Additional methods of use include medical diagnostic, proteomic, and biosensor assays. The multiplexed measurement of quantitative variation in levels of large numbers of proteins allows the recognition of patterns defined by several to many different proteins. The multiplexed identification of large numbers of interactions between proteins and antibodies allows for the recognition of binding and interaction patterns defined by several to many different interactions between proteins and autoantibodies.

The methods and apparatus disclosed herein can be used to screen for various diseases or conditions, including an alteration in the state of the body or of some of the organs, interrupting or disturbing the performance of the functions and/or causing symptoms such as discomfort, dysfunction, distress, or even death to the person afflicted or those in contact with a person. A disease or condition can also include a distemper, ailing, ailment, malady, disorder, sickness, illness, complain, interdisposition and/or affectation.

For example, samples containing antibodies from a diseased animal can be simultaneously screened for the antibodies' ability to interact with a protein on an array. In some embodiments an isotype of antibodies in a sample can be detected. These interactions can be compared to those of samples from individuals that are not in a disease state, not presenting symptoms of persons in the disease state, or presenting symptoms of the disease state. For example, the levels of antigen or ratio of a first antibody/isotype to a second antibody/isotype in samples from a diseased animal can be simultaneously determined. These levels can be compared to those of samples from individuals that are not in a disease state, not presenting symptoms of persons in a disease state, or presenting symptoms of a disease state. In some embodiments, a ratio of an antibody in a disease sample to a healthy sample increasing can be indicative of a disease state. In some embodiments, a ratio of an antibody in a disease sample to a healthy sample decreasing can be indicative of a disease state. In some embodiments a ratio of a first antibody to a second antibody can increase or decrease by about 2×, about 3×, about 4×, about 5×, about 6×, about 7×, about 8×, about 9×, about 10×, about 20×, about 30×, about 40×, about 50×, about 60×, about 70×, about 80×, about 90×, about 100×, about 200×, about 300×, about 400×, about 500×, about 1000×, about 10,000×, about 50,000×, about 100,000×, or about 1,000,000×.

The methods, kits, and compositions described herein can be used in medical diagnostics, drug discovery, molecular biology, immunology and toxicology. Arrays can be used for large scale binding assays in numerous diagnostic and screening applications. The multiplexed measurement of quantitative variation in levels of large numbers of proteins (e.g. proteins) allows the recognition of patterns defined by several to many different proteins, antibody or antibody isotype. The multiplexed identification of large numbers of interactions between proteins and antibodies allows for the recognition of binding and interaction patterns defined by several to many different interactions between proteins and antibodies (including antibody isotypes). Many physiological parameters and disease-specific patterns can be simultaneously assessed. One embodiment involves the separation, identification and characterization of proteins present in a biological sample. For example, by comparison of disease and control samples, it is possible to identify disease specific proteins. These proteins can be used as targets for drug development or as molecular markers of disease.

Detection a level or ratio of one or more proteins or detection of interactions between autoantibodies or antibody isotypes can lead to a medical diagnosis. The sample can be a sample from a subject with a condition or disease. For example, a sample can be a diseased tissue or cell, such as a breast cancer, ovarian cancer, lung cancer (including SCLC or NSCLC), colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer tissue or cell. The sample can be from a subject with a disease or condition such as a cancer, inflammatory disease, immune disease, autoimmune disease, cardiovascular disease, neurological disease, infectious disease, metabolic disease, or a perinatal condition. For example, the disease or condition can be a tumor, neoplasm, or cancer. The cancer can be, but is not limited to, breast cancer, ovarian cancer, lung cancer, colon cancer, hyperplastic polyp, adenoma, colorectal cancer, high grade dysplasia, low grade dysplasia, prostatic hyperplasia, prostate cancer, melanoma, pancreatic cancer, brain cancer (such as a glioblastoma), hematological malignancy, hepatocellular carcinoma, cervical cancer, endometrial cancer, head and neck cancer, esophageal cancer, gastrointestinal stromal tumor (GIST), renal cell carcinoma (RCC) or gastric cancer. The colorectal cancer can be CRC Dukes B or Dukes C-D. The hematological malignancy can be B-Cell Chronic Lymphocytic Leukemia, B-Cell Lymphoma-DLBCL, B-Cell Lymphoma-DLBCL-germinal center-like, B-Cell Lymphoma-DLBCL-activated B-cell-like, or Burkitt's lymphoma. The disease or condition can also be a premalignant condition, such as Barrett's Esophagus. The disease or condition can also be an inflammatory disease, immune disease, or autoimmune disease. For example, the disease may be inflammatory bowel disease (IBD), Crohn's disease (CD), Covid-19, ulcerative colitis (UC), pelvic inflammation, vasculitis, psoriasis, diabetes, autoimmune hepatitis, Multiple Sclerosis, Myasthenia Gravis, Type I diabetes, Rheumatoid Arthritis, Psoriasis, Systemic Lupus Erythematosis (SLE), Hashimoto's Thyroiditis, Grave's disease, Ankylosing Spondylitis Sjogrens Disease, CREST syndrome, Scleroderma, Rheumatic Disease, organ rejection, Primary Sclerosing Cholangitis, or sepsis. The disease or condition can also be a cardiovascular disease, such as atherosclerosis, congestive heart failure, vulnerable plaque, stroke, or ischemia. The cardiovascular disease or condition can be high blood pressure, stenosis, vessel occlusion or a thrombotic event. The disease or condition can also be a neurological disease, such as Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neuropsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The condition may also be fibromyalgia, chronic neuropathic pain, or peripheral neuropathic pain. The disease or condition may also be an infectious disease, such as a bacterial, viral or yeast infection. For example, the disease or condition may be Whipple's Disease, Prion Disease, cirrhosis, methicillin-resistant Staphylococcus aureus, HIV, hepatitis, syphilis, meningitis, malaria, tuberculosis, or influenza. The disease or condition can also be a perinatal or pregnancy related condition (e.g. preeclampsia or preterm birth), zika virus, dengue fever, flavivirus, or a metabolic disease or condition, such as a metabolic disease or condition associated with iron metabolism.

In some embodiments, a detection described herein can be automated. In some embodiments, a detection can comprise detecting multiple disease or conditions simultaneously. In some embodiments, described herein are computers for use in the detection.

Data generated and information obtained by some of the embodiments described herein can be used for a variety of different purposes. In some embodiments, data generated by the methods described herein can provide information on the adaptive immune system of an individual. In some embodiments, information can comprise information regarding infections, foods, allergens, injuries, exercise, microbes, bacteriophages, sex, fetal development, breast milk, an environment of a subject, and other behaviors. In some embodiments, a method described herein can provide information on billions of unique antibody clones in a broad assortment of specialized isotypes and subtypes. In some embodiments, described herein, a method can determine an antigen-specific antibody fingerprint of an individual. In some embodiments, an antigen-specific antibody fingerprint of an individual can be more unique than an individual's genome. In some embodiments, detection and analysis of immunoglobulins using a method as described herein can provide information on a viral and bacterial infection history of a subject. In some embodiments, a significant fraction of an entire human proteome can be targeted by a very unique fingerprint of baseline autoantibodies in healthy individuals. In some embodiments, determination of a preexisting or “natural” baseline autoantibody landscape using a method described herein can provide information regarding an individual's entire prior history of immune experience. In some embodiments, detection of a landscape of autoantibodies can provide information specific to an individual person. In some embodiments, a landscape of autoantibodies can remain stable for many years. In some embodiments, a landscape of autoantibodies can contain unique features reported in association with cancer, autoimmunity, infection, drug toxicity, and neurologic conditions.

In some embodiments, detection of an immune fingerprint can provide information that can have dramatic impacts on human health outcomes. In some embodiments, detection of a natural immune background upon which new responses build, using a method as described herein, can comprise detecting a background of immunity that can even recognize viruses a subject has never been exposed to. In some embodiments, baseline autoantibodies can be detected that can directly manipulate immune signaling. In some embodiments, detection of unique ‘elite’ high-affinity neutralizing autoantibodies to type 1 interferons can comprise detecting IFNα in AIRE-deficient patients. In some embodiments, ‘elite’ high-affinity neutralizing autoantibodies can be neutralizing to cytokine-like molecules. In some embodiments, ‘elite’ high-affinity neutralizing autoantibodies can be lifetime enhancing. In some embodiments, ‘elite’ high-affinity neutralizing autoantibodies can create longer cytokine or other molecule turnover half-life, without neutralizing. In some embodiments, creating longer cytokine or other molecule turnover half-life, without neutralizing can improve their activity, be function enhancing, or a combination thereof. In some embodiments, ‘elite’ high-affinity neutralizing autoantibodies can block or engage receptors as agonists. In some embodiments, detecting naturally occurring IFNα neutralizing autoantibodies in a patient can determine if a patient is protected from type-1 diabetes, a major complication of AIRE-deficiency. In some embodiments, data can show that major changes in global immunity can be created by natural antibodies that inadvertently target and manipulate immunologic pathways. In some embodiments, data generated by methods disclosed herein can suggest that novel drugs and biomarker pathways might be discovered via observing immune manipulation in vivo using methods as described herein.

In some embodiments, antibody fingerprints can be measured using tools such as protein microarrays, peptide microarrays, Luminex libraries, SEREX, nucleotide barcode labelled peptide sequencing, phage display immunoprecipitation sequencing (PhIP-Seq), Rapid Extracellular Antigen Profiling (REAP), mass spectrometry, or any combination thereof.

In some embodiments, disclosed herein are methods that reduce the cost and complexity of surveying antibody fingerprints. In some embodiments, disclosed herein are methods that can provide information on more than a single antibody isotype such as IgG or IgM. In some embodiments, disclosed herein are methods that allow detection of a greater amount of immunologic information comprising underlying isotype and subtype-specific information. In some embodiments, disclosed herein are methods allowing detection of four subtypes of IgG1, IgG2, IgG3, IgG4. In some instances, each subtype of IgG can have dramatically different functionalities. In some embodiments, while all subtypes are typically grouped into a single “IgG” binding score, individual IgG subtypes can have opposite immune effects such as fighting cancer or protecting it. In some embodiments, detection of information regarding IgG can be used to determine rejection of a transplanted organ. For example, in some embodiments, a rejection antigen can be found to depend on IgG2, IgG3, and IgG4, and not IgG1. In some embodiments, IgG4 does not engage complement, and can compete with other subtypes and IgE potentially for tolerogenic effects. In some embodiments, an IgG can be associated with autoimmune diseases in one biological sex more than another. For example, in some embodiments, IgG4-associated disease can be more common in men than women. In some embodiments, detection of an IgG response can comprise information that is useful for understanding the immune response to a virus. For example, detection of a lack of an IgG4 response can provide information that a subject has been exposed to the pathogen responsible for COVID-19. In some embodiments, detection of a lack of IgG4 can potentially provide information related to cross-reactive and associated autoimmune responses.

Disclosed herein are methods and compositions that allow for an increase in the size of the proteome library used to screen for potential autoantibodies. Disclosed herein are methods and compositions that allow for an increase in the number of immunoglobulin isotypes and subtypes included in an assay. Disclosed herein are methods and compositions that allow for data integration of immunoglobulin isotype and subtype data to produce subtype population scores & ratios, including scores involving three or more subtypes. Disclosed herein are methods and compositions that allow for epitope-level integrative data across partner libraries with shorter sequence lengths, such as the universe of known human proteins, infectious viruses, microbiome, bacteriophages, allergens, cancer neoantigens, foods, normal polymorphisms, etc. Disclosed herein are methods and compositions that allow for integration with other known patient-specific factors affecting antibody binding such as genomics, particularly HLA (MHCI/MHCII) genotype in combination with library-wide HLA binding prediction scores.

In some embodiments, disclosed herein are methods comprising: detecting all antibody isotypes on a human protein library simultaneously, and computing ratios, and cross-comparisons of individual immunoglobulin sub-populations. In some embodiments, disclosed herein are methods further comprising combining a full-length protein library data with PhIP-Seq data. In some embodiments, a method can further comprise using a full-length protein library data comprising protein microarray data. In some embodiments, a method can further comprise using protein microarray data comprising human protein microarray data. In some embodiments, a method can further comprise using PhIP-Seq data comprising: human proteome, virome, allergome, mutanome, microbiome, phageome, retrovirome, common polymorphisms, and any combination thereof. In some embodiments, a method can further comprise using an integrated readout of isotype-ratios with epitope-level information from phage-display immunoprecipitation sequencing (PhIP-Seq) for a related protein library (i.e. Human PhIP-Seq). In some embodiments, a method can further comprise combining data with ‘BLAST’ type cross comparisons, to back-trace cross reactive antigens from their origin in virus or microbe to isotype-ratio immune response. In some embodiments, a method can further comprise combining data with another patient-specific multi-omic integration. In some embodiments, another patient-specific multi-omic integration can comprise: HLA-binding algorithms, genomic sequencing, T cell receptor/B cell receptor sequencing, and any combination thereof. In some embodiments, a T cell receptor/B cell receptor sequencing can predict likelihood of corresponding T cell responses based on these ratios and backtrack them to individual parent cell clones. In some embodiments, a method can further comprise combining data with known ontology databases.

In some embodiments disclosed herein a method can comprise detecting more than two antibody isotypes across a human protein library simultaneously and integrating this information with other data, including up to all known human isotypes in a single assay (IgG1, IgG2, IgG3, IgG4, IgA1, IgA2, IgD, IgE, IgM). In some embodiments, a ratio of antibody isotype can comprise an IgG1/IgG3, IgG1/IgA2, or IgG4/IgE ratio or a combination thereof. This may additionally include multimer variants such as IgA1 monomer and IgA1 dimer. In some embodiments, a method can further comprise computing ratios and cross-comparisons of individual immunoglobulin sub-populations involving two or more antibody isotypes or subtypes to derive highly-specific specific subpopulations not previously identifiable (IgG1-negative, IgG2-mid, IgG3-high, IgGE-high); this optionally involves direct quantification of individual isotypes and subtypes globally from a patient to better help with normalization and cross-comparison. In some embodiments, a ratio can be used to score antigen-specific antibody responses according to individual interacting combinations of immune cell subpopulations known to bear specific Fc receptors (i.e. pro-tolerance, pro-effector, anti-allergen, pro-IgG4-specific disease, anti-viral, etc). In some embodiments, information can be used to create novel ways of diagnosing, stratifying, or treating disease, particularly conditions which are ‘inflammatory’ such as cancer, known autoimmune diseases, neurodegenerative disease, other neurologic conditions such as autism, epilepsy, and schizophrenia, healthy ageing, unhealthy ageing, cardiovascular disease, obesity, diabetes, survival or prevention of infection such as COVID-19, HIV, food intolerances, allergies, bacterial infections, etc. In some embodiments, stratifying a disease can comprise identifying a subgroup of patients with distinct mechanisms of disease or responses to treatment. In some embodiments, identifying a distinct mechanism of disease or response to treatment can allow for a personalized treatment approach. In some embodiments, proposed novel therapies may involve novel antigen-specific CAR-T/CAR-B cell treatments, monoclonal antibodies and antibody derivatives, BiTEs, DARTs, tolerogenic vaccines, effector vaccines, gene therapy, etc. In some embodiments, therapies can comprise effector therapies (i.e. vaccine, CAR-T) to prevent infection or induce healthy autoimmunity (i.e. ageing prevention via senescent cell elimination or cancer prevention via tumor cell elimination). In some embodiments a therapy can comprise tolerance & prevention therapies (i.e. vaccine, CAR-Treg) to halt harmful autoimmunity or inflammation of as-yet unknown cause (i.e. prevention of depression, obesity, cardiovascular disease, diabetes, organ failure, solid organ transplant, bone marrow transplant, neurodegeneration, muscle degeneration, allergies, food intolerances, etc.). In some embodiments, a method can further comprise incorporating multiple titrations in order to separate antibody abundance from affinity from abundance in the context of the overall assay. In some embodiments, a method can further comprise incorporating an integrated readout of isotype-ratios with epitope-level information from companion antibody detection technologies covering a similar human autoantigen library using shorter sequences (i.e. peptide array or phage-display immunoprecipitation sequencing (PhIP-Seq)) to uncover a ‘real autoantigen’ when there are multiple-related hits within a longer-sequence parent library (i.e. autoimmunity to which protein within a family with shared subdomains causes the disease. In some embodiments, this can be observable via ‘epitope spreading’ where a real target has more hits in an epitope-level library than in ‘off target’ proteins). In some embodiments, a method can further comprise incorporating an integrated readout of isotype ratios with information from companion antibody detection technologies covering a different library. In some embodiments a library can comprise human viruses, allergens, mutations, microbiome, foods, bacteriophages, common polymorphisms, normal retroviruses, etc., along with sequence-relationship cross-comparisons (i.e. NCBI BLAST), to back-trace to find an ‘original offending antigen’ (i.e. in a virus or microbe) that results in a disease-causing multi-isotype cross-reactive autoantigen response (i.e. viral caused autoimmunity or cancer). In some embodiments, a method can further comprise combining a method disclosed herein with other patient-specific multi-omic integrations, such as HLA-binding algorithms, genomic sequencing, and T cell receptor/B cell receptor sequencing to predict likelihood of corresponding T cell responses based on these ratios and determining the individual parent cell clones. In some embodiments, a method can further comprise combining a method disclosed herein with T cell receptor and B cell receptor sequencing to use new antibody fingerprint data to identify specific-corresponding T and B cell responses based on these ratios and determining the individual parent cell clones for use in diagnostics (i.e. public TCRs) or therapies (i.e. CAR-T, monoclonal antibodies). In some embodiments, a method can further comprise using a method described herein with known ontology databases to identify specific cellular pathways which may provide deeper information.

In some embodiments, disclosed herein, are methods of profiling one or more antibodies, using a microarray as described herein. In some embodiments, a method can comprise using a pan-isotype, entire proteome microarray as described herein. In some embodiments, a microarray can comprise probes for peptides instead of proteins. In some embodiments, a microarray can comprise antiviral antibodies to a phage library. In some embodiments, a phage library can comprise peptides from 206 species and over 1000 strains of viruses. In some embodiments, all viruses are annotated to have human tropism in the UniProt database.

In some embodiments, a microarray can comprise a slide. In some embodiments, a method of antibody profiling can comprise slide scanning of a slide comprising a microarray. In some embodiments, a microarray and slide scanning can be used to provide data on 3D non-continuous epitopes. In some embodiments, data on 3D non-continuous epitopes can't be obtained in a peptide-based analysis system. In some embodiments, a method can be used to provide multi-isotype profiling and other secondary binders like oligonucleotides.

In some embodiments, a human proteome microarray can be used to provide protein data. In some cases, protein data can comprise well-folded protein with conformationally intact epitopes. In some embodiments, a method can comprise detecting primary (IgM) and secondary (IgG) responses (and other Igs). In some embodiments, a method can comprise an assay with rapid and reproducible results. In some embodiments, a method can comprise a total time of about 1, about 2, about 3, about 4, about 5, about 6, about 7, about 8, about 9, about 10, about 11, or about 12 days. In some embodiments, a method can comprise a total time of about 1, about 2, about 3, about 4, about 5, about 6, about 7, about 8, about 9, about 10, about 11, or about 12 weeks.

In some embodiments, a method can comprise using a volume of sample. In some embodiments, a volume of sample used can comprise about 1 μl, about 2 μl, about 3 μl, about 4 μl, about 5 μl, about 6 μl, about 7 μl, about 8 μl, about 9 μl, about 10 μl, of serum or plasma per assay. In some embodiments, a method can comprise use of 50-100 samples for each category. In some embodiments, a method can comprise washing a microarray and incubating with isotype-specific fluorescent secondary antibodies. In some embodiments, isotype-specific fluorescent secondary antibodies can comprise IgM, IgM B-cell receptor, IgM pentamer, IgG, IgG1, IgG2, IgG3, IgG4, IgA, IgA1 secreted IgA1 dimer, IgA2, secreted IgA2 dimer, IgD, IgD B-cell receptor, IgE, and any combination thereof.

In some embodiments, a microarray can comprise a peptide library. In some embodiments, a peptide library can comprise a UniProt library of human proteins. In some embodiments, a UniProt library of human proteins can comprise 24,239 individual ORFs. In some embodiments, a library can be divided into 259,345 90mer peptide tiles with 45 residues overlap. In some embodiments, a peptide library can comprise about 200mer peptide tiles, about 150mer peptide tiles, about 100mer peptide tiles, about 90mer peptide tiles, about 80mer peptide tiles, about 70mer peptide tiles, about 60mer peptide tiles, about 50mer peptide tiles, about 40mer peptide tiles, about 30mer peptide tiles, about 20mer peptide tiles, about 15mer peptide tiles, about 10 mer peptide tiles, or about 5mer peptide tiles. In some embodiments, a peptide library can comprise a UniProt library of all viruses annotated to have human tropism, comprising about 206 species and about 1000 strains. In some embodiments, a programmable DNA microarray can be used to encode peptides as synthetic oligonucleotides. In some embodiments, T7 phage library construction can be performed. In some embodiments, serum screening can be performed. In some embodiments, serum screening can comprise incubating a library with a serum sample containing antibodies. In some embodiments, antibodies can be immunoprecipitated using protein A/G coated magnetic beads, and unbound phage removed by washing. In some embodiments, a library of peptide encoding DNA sequences can be amplified by PCR directly from an immuno-precipitate. In some embodiments, a sample-specific barcode can be added with a second round of hemi-nested PCR. In some embodiments, massively parallel sequencing of phage DNA can be performed to quantify enrichment of each library member due to antibody binding. In some embodiments, results can be used to generate an individual or population autoantibody profile.

In some embodiments, a method can comprise combining data obtained from a method of immunoprofiling or antibody screening on a human proteome microarray, and a method of antibody screening on a viral protein array. In some embodiments, a human proteome detection can comprise a microarray. In some embodiments, a microarray can comprise a pan-isotype proteome-wide array. In some embodiments, a human proteome detection can comprise a peptide microarray, a Luminex library, SEREX, nucleotide barcode labelled peptide sequencing, phage display immunoprecipitation sequencing (PhIP-Seq), a direct sequencing, other nucleotide barcoded libraries & readout methods, Rapid Extracellular Antigen Profiling (REAP), mass spectrometry and any combination thereof. In some embodiments, a mass spectrometry can comprise LC-MS mass spectrometry. In some embodiments, a direct sequencing can comprise direct protein sequencing.

In some embodiments, a method can comprise a rapid turnaround and comprehensive reporting. In some embodiments, a method can produce results comprising quantitative raw data, and quantile normalized outputs. In some embodiments, a method can produce results with overall case-control statistics provided. In some embodiments, a method can comprise use of bioinformatic analysis of array scanning results, metadata, or a combination thereof.

In some embodiments, a method can comprise combining data from a method of immunoprofiling or antibody screening on a human virome microarray. In some embodiments, a human proteome microarray can comprise a peptide microarray, a peptide library with antigens to infectious viruses or bacteria, foods, allergens, bacteriophages, or other environmental exposures, and any combination thereof.

Methods, devices and kits provided herein can assess a condition, disease, responsiveness to treatment or responsiveness to a disease by a subject with high specificity and sensitivity. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, devices and kits provided herein can assess a condition in a subject with a specificity of at least about 75%, 80%, 85%, 90%, 95%, 96%, 97%, 98%, 99%, or 100%. Methods, devices and kits provided herein can assess condition, disease, responsiveness to treatment or responsiveness to a disease by a subject with a sensitivity of at least about 10%, 15%, 20%, 25%, 30%, 35%, 40%, 45%, 50%, 55%, 60%, 70%, 75%, 80%, 85%, 90%, 95%, 96%, 97%, 98%, 99%, or 100%. Methods, devices and kits provided herein can assess a condition, disease, responsiveness to treatment or responsiveness to a disease by a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods of assessing a condition, disease, responsiveness to treatment or responsiveness to a disease of subject herein can achieve high specificity and sensitivity based on the expression of one or more antibodies or antibody isotypes. In some cases, the methods of assessing a condition, disease, responsiveness to treatment or responsiveness to a disease by a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the expression of no more than 2, 3, 4, 5, 6, 7, 8, 9, or 10 biomarkers. In some cases, the methods, devices and kits of assessing a condition, disease, responsiveness to treatment or responsiveness to a disease by a subject can achieve a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the expression of two biomarkers.

An array described herein can comprise an ordered spatial arrangement of two or more proteins, antibody or antigen, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 proteins, antibody or antigen. An array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for a protein. The proteins can be linked to the array by the antibodies. Thus, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 proteins linked to the array by at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 antibodies specific for the proteins.

An array can comprise an ordered spatial arrangement of two or more same or different proteins, on a solid surface. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different proteins. For example, an array can comprise at least 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 same or different proteins.

An array can be a high-density array. A high-density array can comprise tens, hundreds, thousands, tens-of-thousands or hundreds-of-thousands of proteins. The density of microspots of an array may be at least about 1/cm² or at least about 10/cm², up to about 1,000/cm² or up to about 500/cm². In certain embodiments, the density of all the microspots on the surface of the substrate may be up to about 400/cm², up to about 300/cm², up to about 200/cm², up to about 100/cm², up to about 90/cm², up to about 80/cm², up to about 70/cm², up to about 60/cm², or up to about 50/cm². For example, an array can comprise at least 50, 60, 70, 80, 90, 100, 150, 200, 250, 300, 350, 400, 450, 500, 600, 700, 800, 900, or 1,000 distinct antibodies per a surface area of less than about 1 cm². For example, an array can comprise 50, 60, 70, 80, 90, 100, 150, 200, 250, 300, 350 or 400 discrete regions in an area of about 16 mm², or 2,500 discrete regions/cm². In some embodiments, proteins, linkers, or another moiety in each discrete region are present in a defined amount (e.g., between about 0.1 femtomoles and 100 nanomoles). For example, an array can comprise at least about 2 proteins per cm². For example, an array can comprise at least about 2, 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more proteins, antigens, or antibodies. For example, an array can be a high-density protein array comprising at least about 10 proteins, antibody, or antigen per cm². For example, an array can comprise at least about 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, or more proteins, antigens, or antibodies per cm².

Disclosed herein in some embodiments, are compositions comprising a microarray. In some embodiments, a microarray can allow simultaneous detection of autoantibodies to any of at least about 21,000 full length, properly folded, human proteins. In some embodiments, a properly folded human protein can comprise an antigen. In some embodiments, a microarray can comprise more than 21,000 unique human proteins, isoform variants, and protein fragments. In some embodiments, a microarray can cover 16,794 unique genes. In some embodiments, a microarray can comprise 15,889 of the 19,613 canonical human proteins described in the Human Protein Atlas, with broad coverage across protein subclasses. In some embodiments, a microarray can comprise probes that detect the majority of the human proteome. In some embodiments, a microarray can comprise probes that detect about all of the human proteome. In some embodiments, a microarray can comprise probes that detect the entire proteome. In some embodiments, a microarray can comprise probes that detect Immunoglobulin (Ig). In some embodiments, a microarray can comprise probes that detect IgM, IgM B-cell receptor, IgM pentamer, IgG, IgG1, IgG2, IgG3, IgG4, IgA, IgA1 secreted IgA1 dimer, IgA2, secreted IgA2 dimer, IgD, IgD B-cell receptor, IgE, and any combination thereof. In some embodiments, via altered secondaries, a microarray can be capable of detecting binding of at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, of all Ig isotypes or subtypes. In some embodiments, via altered secondaries, a microarray can be capable of detecting binding of any Ig isotype or subtype. In some embodiments, determination of an isotype or subtype of an antibody can determine whether an antibody can be cytotoxic, tolerogenic, or can help educate T cells. In some embodiments, a human proteome microarray can be used for pan-isotype autoantibody biomarker profiling versus properly folded, three-dimensional human proteins.

In some embodiments, use of a proteome-wise microarray can allow detection of previously undiscovered antigens. In some embodiments, detection of previously discovered antigens can play a role in human disease. In some embodiments, once discovered a target can be used as a novel biomarker to diagnose a disease. In some embodiments, once discovered a target can be used as a novel biomarker to determine a treatment. In some embodiments, once discovered a target can lead to a new therapeutic or vaccine. In some embodiments, a target detected using a method herein can determine a treatment course. In some embodiments, a treatment course can be personalized to a subject using data derived from methods disclosed herein. In some embodiments, disclosed herein is a system, device, or combination thereof capable of performing a method disclosed herein to generate a data. In some embodiments, a device can transmit a data to another device.

In some embodiments, a microarray can comprise probes for a peptide library. In some embodiments, a peptide library can comprise a full UniProt human proteome (build 35.1) presented as overlapping peptides via a T7 phage display library. In some embodiments, a library can be reacted with patient antibodies, bound phages pulled down with protein A/G, and sequenced for results. In some embodiments, a peptide library can comprise 24,239 ORFs. In some embodiments, a peptide library can comprise 90-residue peptide tiles; with 45-residues overlapping. In some embodiments, a peptide library can comprise about 200mer peptide tiles, about 150mer peptide tiles, about 100mer peptide tiles, about 90mer peptide tiles, about 80mer peptide tiles, about 70mer peptide tiles, about 60mer peptide tiles, about 50mer peptide tiles, about 40mer peptide tiles, about 30mer peptide tiles, about 20mer peptide tiles, about 15mer peptide tiles, about 10mer peptide tiles, or about 5mer peptide tiles.

Disclosed herein in some embodiments, are compositions comprising microarrays that can present the entire UniProt human proteome and UniProt viruses with human tropism. In some embodiments, a microarray can present the entire UniProt human proteome and UniProt viruses with human tropism as long peptides on bacteriophages. In some embodiments, a microarray can provide epitope-level data on patient antibodies versus at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99%, of all known linear viral and human autoantigens. In some embodiments, a microarray can be limited to IgG detection and cannot provide isotype or subtype-level data.

In some embodiments, a microarray can comprise probes for a peptide library. In some embodiments, a peptide library can comprise all UniProt viral proteins with human tropism presented as overlapping peptides via a T7 phage display library. In some embodiments, a library can be reacted with patient antibodies, bound phages can be pulled down with protein A/G and sequenced for results. In some embodiments, a peptide library can comprise 206 species comprising more than 1000 strains in 56-residue peptide tiles with 28-residues overlapping.

In some embodiments, a microarray can be manufactured by mobilizing an ORF collection into a yeast galactose-inducible GST fusion vector (pEGH-1A) using Gateway-mediated site-specific recombination. In some embodiments, each clone can be individually verified to have correct inserts by both BsrGI digestion and plasmid DNA sequencing. In some embodiments, a number of clones individually verified can comprise >21,000 clones. In some embodiments, clones with confirmed size, sequence and high copy number can be preserved in a library. In some embodiments, a library can be routinely transformed into yeast for large-scale protein expression and N-terminal GST tag purification.

In some embodiments, a piezoelectric “inkjet” process can be used to manufacture microarrays. In some embodiments, a piezoelectric “inkjet” process can be used to allow rapid production of high quality microarray slides. In some embodiments, a piezoelectric “inkjet” process can be used to improve accuracy and reproducibility. In some embodiments, a piezoelectric “inkjet” process can be used to produce excellent spot morphology with even pixel distribution.

In some embodiments, a non-contact piezoelectric (inkjet) printer can be used to manufacture microarrays. In some embodiments, a non-contact piezoelectric (inkjet) printer can be used to manufacture microarrays with significant performance advantages over contact print methods used in older-generation protein microarrays. In some embodiments, significant performance advantages can include: precise spot morphology, up to 1000 array print batch sizes for large cohort analysis, rapid production of custom control configured arrays, reduced inter- and intra-array variability, greater data reproducibility.

After purification, GST-tagged proteins can be piezoelectrically printed on glass slides in duplicate, along with control proteins. In some embodiments, control proteins can include GST, BSA, Histones, IgG, and any combination thereof. In some embodiments, slides can be barcoded for tracking and archiving. In some embodiments, each microarray batch can be routinely evaluated by anti-GST staining to demonstrate quality of expression and printing. In some embodiments, slides can comprise nitrocellulose or an epoxy resin.

Also provided are kits that find use in practicing the subject methods, as mentioned above. A kit can include one or more of the compositions described herein. A kit can include at least one protein. A kit can include at least one antibody. A kit can comprise barcoded proteins.

A kit can include a solid support. In some embodiments, the solid support is already functionalized with at least one protein. In some embodiments, the solid support is not functionalized with at least one protein. A kit can include a reagent for coupling at least one protein to the solid support.

A kit can include one or more reagents for performing amplification, including suitable primers, enzymes, nucleobases, and other reagents such as PCR amplification reagents (e.g., nucleotides, buffers, cations, etc.), sequencing and the like. Additional reagents that are required or desired in the protocol to be practiced with the kit components may be present. Such additional reagents include, but are not limited to, one or more of the following an enzyme or combination of enzymes such as a polymerase, reverse transcriptase, nickase, restriction endonuclease, uracil-DNA glycosylase enzyme, enzyme that methylates or demethylates DNA, endonuclease, ligase, etc.

As indicated above, certain protocols will employ two or more different sets of such probes for simultaneous detection of two or proteins in a sample (e.g., in multiplex and/or high throughput formats). In some embodiments a kit includes two or more distinct sets of antibodies, and/or proteins.

The kit components may be present in separate containers, or one or more of the components may be present in the same container, where the containers may be storage containers and/or containers that are employed during the assay for which the kit is designed.

In addition to the above components, the subject kits may further include instructions for practicing the subject methods. These instructions may be present in the subject kits in a variety of forms, such as printed information on a suitable medium or substrate (e.g., a piece or pieces of paper on which the information is printed), in the packaging of the kit, in a package insert, etc. Yet another means would be a computer readable medium (e.g., diskette, CD, etc.), on which the information has been recorded. Yet another means that may be present is a website address which may be used via the internet to access the information at a removed site.

Additional embodiments of the invention relate to the communication of assay results or diagnoses or both to technicians, physicians or subjects, for example. In certain embodiments, computers will be used to communicate results of the assessing or diagnoses or both to interested parties, e.g., physicians and their subjects. In some embodiments, the assessing can be performed, or results analyzed in a country or jurisdiction which differs from the country or jurisdiction to which the results or diagnoses are communicated. In some embodiments of the invention, a diagnosis based on the presence or absence in a test subject of any biomarker identified by the invention may be communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test maybe generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

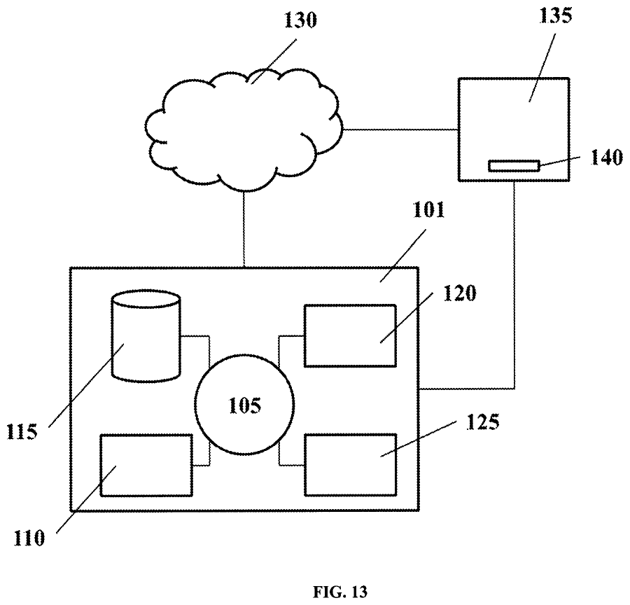

The present disclosure provides computer control systems that are programmed to implement methods of the disclosure. FIG. 13 shows a computer system 101 that is programmed or otherwise configured to interface with a sequence library, a sequencer, a PCR machine, an array, an apparatus that is configured to sequence, amplify or analyze an oligonucleotide, an antibody, a protein, a substrate, or any combination thereof. The computer system 101 can regulate various aspects of the present disclosure. The computer system 101 can regulate amplification conditions, associating conditions, sequencing conditions, detection conditions, such as buffer types, temperatures, or time periods of incubation. The computer system 101 can be an electronic device of a user or a computer system that is remotely located with respect to the electronic device. The electronic device can be a mobile electronic device.

The computer system 101 can include a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 can be in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases can be a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries, protein library data, detection data and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, one or more results (immediate results or archived results from a previous experiment), one or more user inputs, reference values from a library or database, or a combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 105. The algorithm can, for example, determine optimized conditions via supervised learning to optimize conditions such as a buffer type, a buffer concentration, a temperature, an incubation period. Conditions may be optimized for an oligonucleotide fragment, such as an oligonucleotide fragment having a particular number of epigenetic modifications or a particular length of sequence.

Disclosed herein in some embodiments, are methods of classifying individual antigen-specific antibody binding. In some embodiments, a method of classifying individual antigen-specific antibody binding can comprise an immunoassay. In some embodiments, an immunoassay can comprise a pan-isotype immunoassay. In some embodiments, an immunoassay can comprise a proteome-wide immunoassay. In some embodiments, an immunoassay can classify individual antigen-specific antibody binding. In some embodiments, individual antigen-specific antibody binding can be classified according to its isotype or subtype. In some embodiments, individual antigen-specific antibody binding can be associated within a broader context of binding scores for other antibody isotypes, subtypes, ratios, and more complex antibody population subdivisions.

### EXAMPLES

### Example 1: Proteome-Wide Pan-Isotype Autoantibody Immunoprofiling of a Healthy Human Male

Serum was collected from a healthy adult human male donor, incubated on human proteome microarrays, and stained with a pair of anti-isotype secondaries. The full ten channel analysis was scanned, quantile normalized, and log 2 transformed. Z-scores were computed for autoantibody binding versus each of >21,000 individual protein targets for outlier antigen binding within the cohort. Z-scores were then plotted in binned density plots to observe the frequency distribution of antigen-specific autoantibody binding overlap across isotype pairs (above, blue=1 antigen overlap; red=>130 antigen overlap).

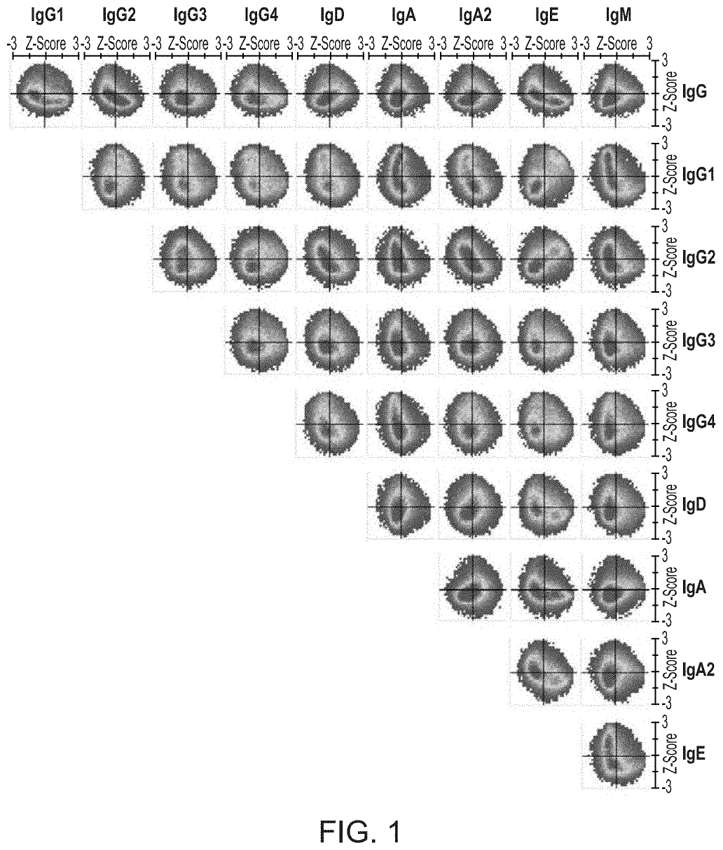

In the normalized data shown in FIG. 1, dramatic population-level associations were observed across various isotype pairs, suggesting population-wide antigen-specific isotype and sub-isotype interrelationships exist across the universe of human autoantigens. These data suggest an integrative method could elucidate novel antigen-specific autoantibody interrelationships between specific antibody isotypes, subtypes, and isotype ratios in the context of wider multi-omic data. Additional value could be gained from epitope-level autoantigen and viral antigen data in the IgG channel alone by PhIP-Seq.

As shown in FIG. 1, there was no proteome-wide antigen-specific interrelationship between some of the pairs (i.e.: IgG4 vs IgG3), whereas others demonstrated positive relationships (IgG2 vs IgG1), and others inverse relationships (IgM vs IgE). Interestingly IgEhi overlapped with IgG1hi, and IgEmid overlapped with IgG2mid. These data suggest information exists in isotype-level relationships which is not typically captured in global IgG protein microarray analyses.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 2: Reproducible Proteome-Wide IgG and IgA Autoantibody Immunoprofiling of a Healthy Human Male within and Across HuProt Proteome Microarray Batches Demonstrates Reliability of Multi-Array Analysis

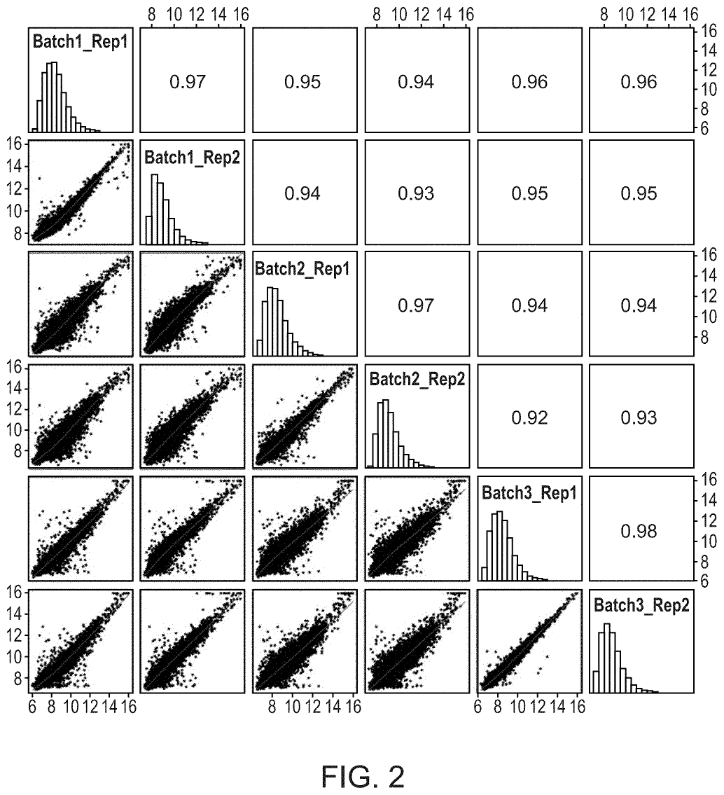

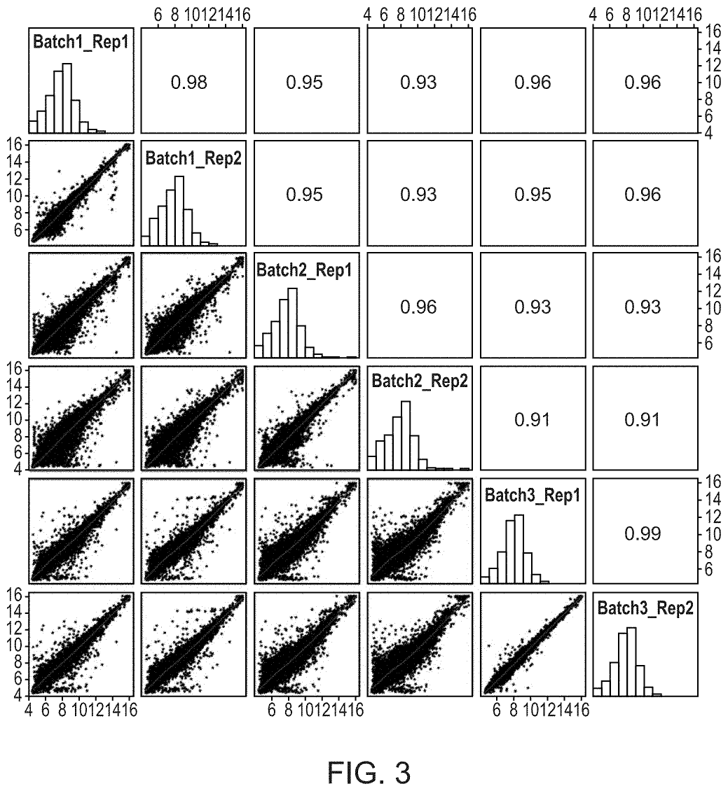

Serum was collected from a healthy adult human male donor, incubated on pairs (Rep1, Rep2) of HuProt proteome microarrays across three print batches (Batch 1; Feb. 12, 2020, Batch 2; Dec. 9, 2019, Batch 3; Oct. 1, 2010), and stained with anti-IgG (A., 635 nm, Red, FIG. 2) and anti-IgA (B., 532 nm, Green, FIG. 3) secondaries. Arrays were scanned and raw fluorescence intensity data was plotted on a log scale and linear regression analysis was performed. Intra-lot correlations of spot pair averages (Rep 1 vs Rep 2; intra-lot) was >0.95 R\hat{ }2 within all three batches in both channels. Slide-to slide cross pairings across all possible pairs of the six slides was a >0.90 R\hat{ }2 correlation—demonstrating robust reproducibility of HuProt microarray data between any individual slide; these results demonstrate multi-sample, multi-batch, or multi-isotype analysis requiring multiple slides should be reliable.

### Example 3: Demonstration of Differential Antibody Affinity Populations by Proteome Array Titration in Serum from a Healthy Human Male

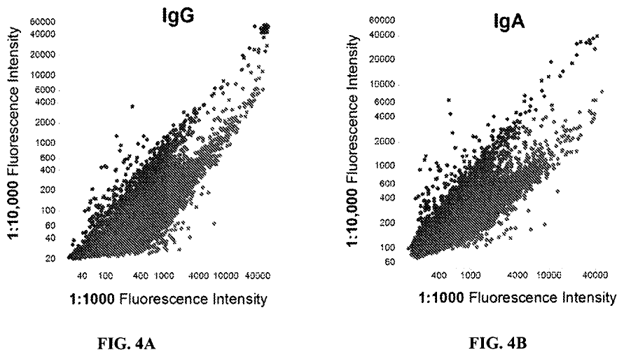

Serum was collected from a healthy adult human male donor, incubated on pairs of human proteome microarrays as described herein at dilutions of 1:1,000 and 1:10,000. Arrays were stained with anti-IgG and anti-IgA secondaries and scanned for fluorescence intensity measurements. Raw data were plotted on a log scale and colored red according to signal preservation after dilution as shown on FIG. 4A for IgG, and FIG. 4B for IgA. Red signals remaining on the center line demonstrate higher-affinity antibodies that preserved their signal at 1:10,000 dilution, whereas blue signals shifted down demonstrate a lower-affinity subpopulation. These results demonstrate the value of dose titrations for differentiating higher affinity autoantibody subpopulations from lower affinity autoantibody subpopulations.

### Example 4: Multi-Dimensional Antibody Discovery

Patient antibodies are incubated on a library of autologous human proteins in some format such as a protein microarray. Patient antibodies are allowed to bind, and bound antibodies are stained or otherwise quantified in some format such as with fluorescent secondary antibodies and a microarray scanner using one or multiple slides. These antibodies are stained and quantified for not one, but three or more—often all—known human antibody isotypes and subtypes simultaneously. This may involve multiple titrations (FIG. 3 and FIG. 4) in order to separate ‘abundant’ from ‘high affinity’ antibodies, which may result in different populations and fingerprint scores. These values are then normalized for cross-comparison, potentially involving direct quantification of the percent circulating serum antibody isotype and subtypes. These data are combined into a multi-dimensional readout of the antibody fingerprinting, which also involves epitope-level antibody data from the same sample tested against other protein libraries. This data can be useful for deriving the origin of the specific autoantibodies (i.e. via a cross-reactive infectious virus) as well as the specific autologous target via using smaller peptides than the parent library (i.e. in the case of conserved protein domains, the ‘real’ antigen target might have multiple epitope-level hits, whereas irrelevant hits might only bind the cross-reactive epitope).

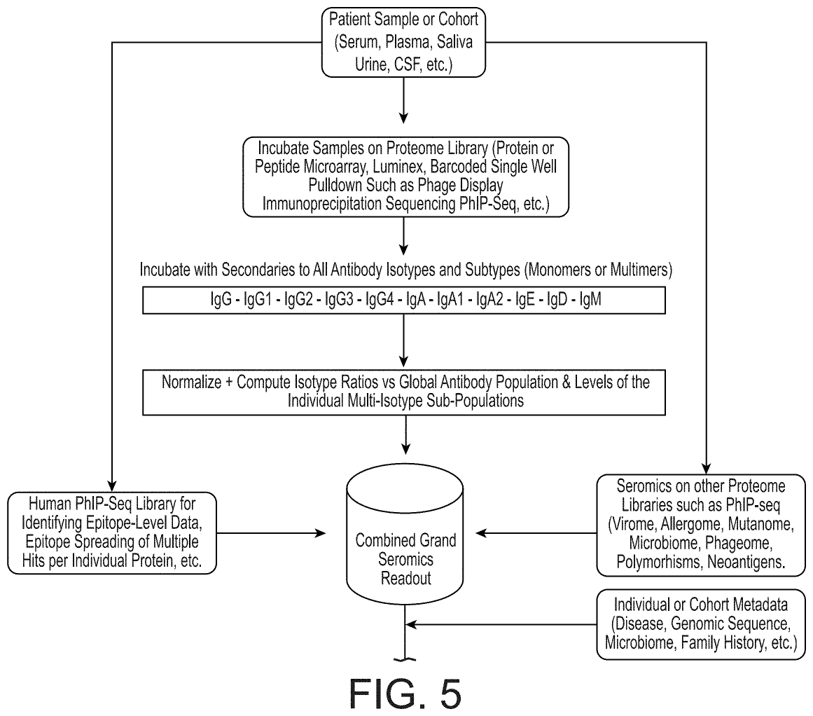

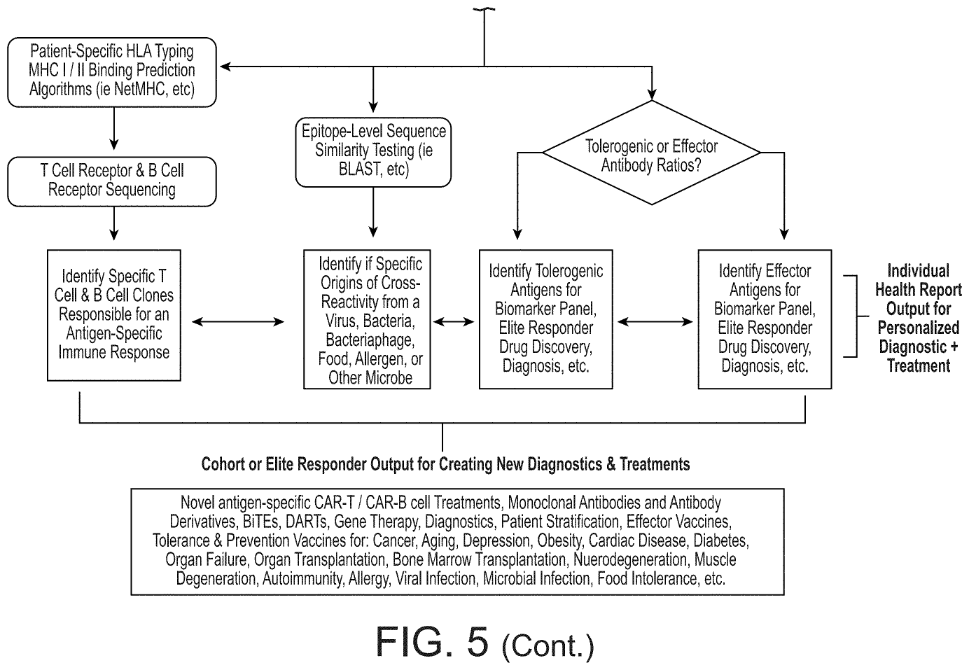

To make use of this antibody fingerprint or multi-dimensional readout, we introduce additional metadata or multi-omic information on the patient or sample cohort. Several independent analyses then occur—each of which requires this parent antibody fingerprint data. A schematic overview is shown in FIG. 5.

Identifying specific T or B cell clones responsible for initiating some specific antigen-level response detected in the fingerprint data. This is done by combining HLA typing for WWI/II with MEW binding prediction algorithms across both the parent multi-isotype library and the accessory shorter-epitope (i.e. viral PhIP-Seq) library. This is particularly useful for using elite responders, healthy agers, disease survivors (i.e. COVID-19) to identify novel receptors or antibodies to use in therapeutics such as monoclonal antibodies or CAR-T cells.

In some instances, this data may be used in identifying a specific cross-reactive origin of offending antigens in a particular disease (i.e.: HPV virus causes cervical cancer) to identify the food, virus, bacteria, allergen, etc. responsible for a specific condition which has yet to be fully elucidated. In some instances, this can potentially be applicable to nearly all chronic non-mendelian diseases (i.e.: ageing, ALS, Alzheimer's, autism, schizophrenia, epilepsy, cardiovascular disease, other neurologic disorders and neurodegenerative disease, depression, obesity, diabetes, organ transplant, death or complications from viral infection. In some instances, the data can be used to determine answers to questions such as why only some tuberculosis patients acquire chronic disease, or why only some COVID-19 patients die and why that overlap corresponds to patient populations (i.e. older men) most likely to get IgG4 immune disease.

In some instances, it may be possible to derive new information on whether an individual multi-isotype response means something more related to immune tolerance or effector function. This can be visualized as an antibody fingerprint similar to flow-cytometry frequency distribution plots, as shown in FIG. 1, (i.e. IgG1-negative, IgG2-mid, IgG3-high, IgGE-high) in order to identify novel biomarker populations for diagnostics, stratification, or drug discovery by elite responders or disease avoiders.

These data can be combined to create individualized health reports, such as giving a patient information on whether they are likely to acquire Alzheimer's disease based on their immune history, give birth to a child with autism, or need a particular medical intervention upon infection with a virus such as SARS-CoV-2 (COVID-19), etc. Across cohorts of patients, or using elite responding or surviving patients, this information can also be used to identify new diagnostic methods or treatments tailored to this fingerprint information, such as aging, neuroprotection, autism-prevention, cancer-prevention, and viruses such as SARS-CoV-2 (COVID-19), etc.

### Example 5: Increase in PubMed Publications Including the Term ‘Inflammatory’ in the Abstract Across Different Disease Conditions

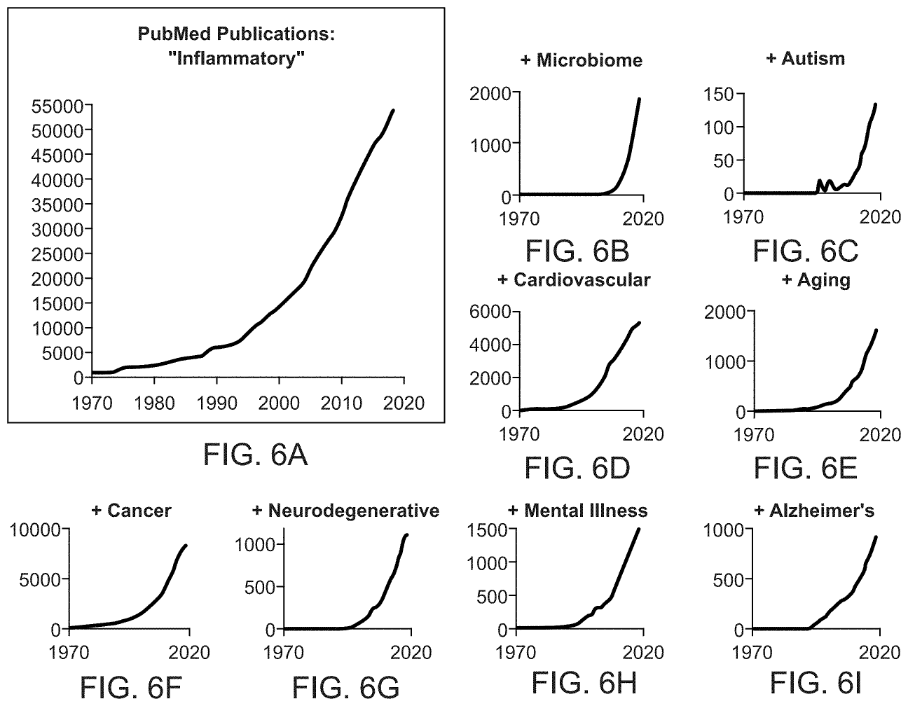

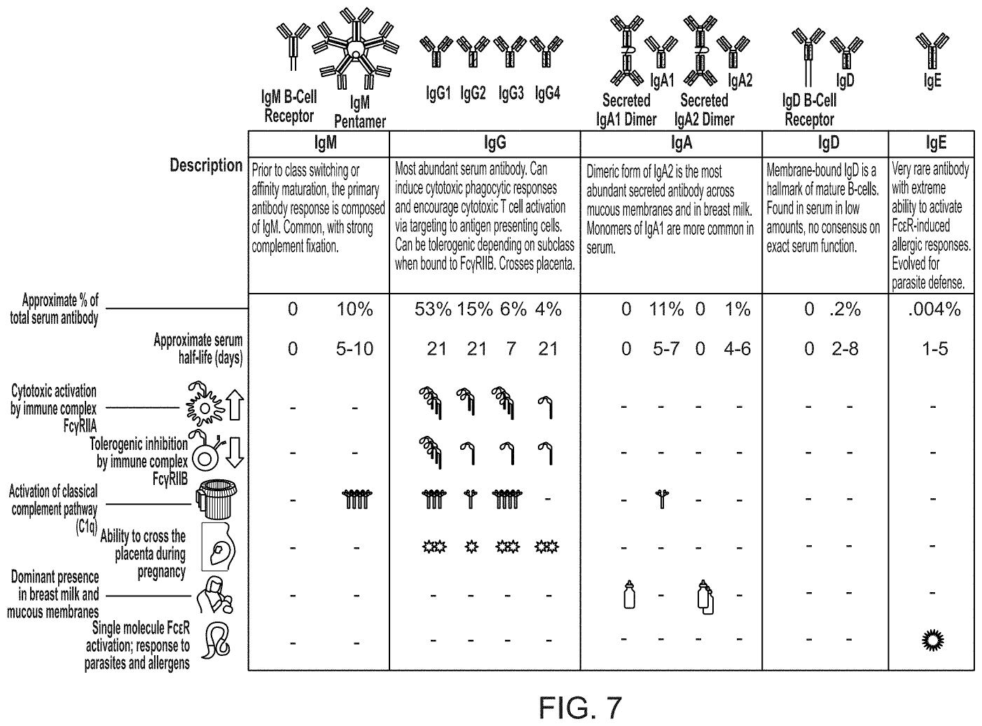

Many of humanity's greatest unsolved medical mysteries are increasingly recognized as being “inflammatory” conditions, as shown in FIG. 6A. These conditions also include, those associated with FIG. 6B microbiome, FIG. 6C autism, FIG. 6D cardiovascular, FIG. 6E aging, FIG. 6F cancer, FIG. 6G neurodegenerative, FIG. 6H mental illness, and FIG. 6I Alzheimer's. While an inflammatory response often involves a specific antigen—most of the offending antigens in so called ‘inflammatory’ diseases remain unidentified. In fact, many ‘inflammatory’ diseases place blame on cellular function, and not antigen-specific immunity (i.e. a link between Alzheimer's and tau and tangles production). They might now be identified with the methods described here (i.e. presence of tau and tangles in Alzheimer's is an antigen-specific immune response identifiable by antibody fingerprinting which results in tau and tangles). Additionally, death from viral or bacterial infection may not be due to the infection itself—but the way that the immune system decides to respond to it, including collateral auto-immune damage. FIG. 7 shows some of the known function and diversity of individual antibody isotypes and subtypes.

### Example 6: Human Proteome Microarray

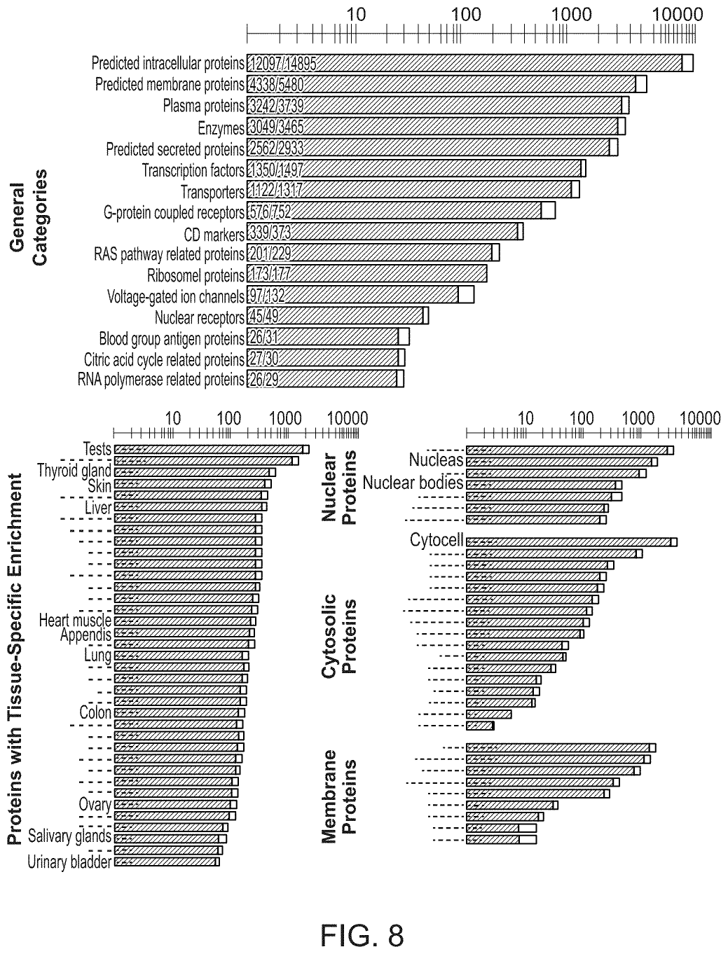

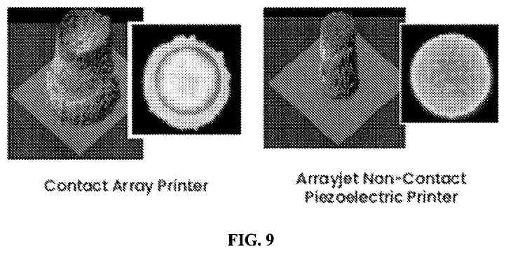

A human proteome microarray was manufactured containing all >21,000 unique human proteins, isoform variants, and protein fragments—covering 16,794 unique genes. This includes 15,889 of the 19,613 canonical human proteins described in the Human Protein Atlas, with broad coverage across protein subclasses. FIG. 8 depicts a graph showing the numbers of proteins of each category in the array. FIG. 9 depicts an image showing a more precise spot morphology produced by a manufacturing method using a non-contact piezoelectric printer compared to a contact array printer.

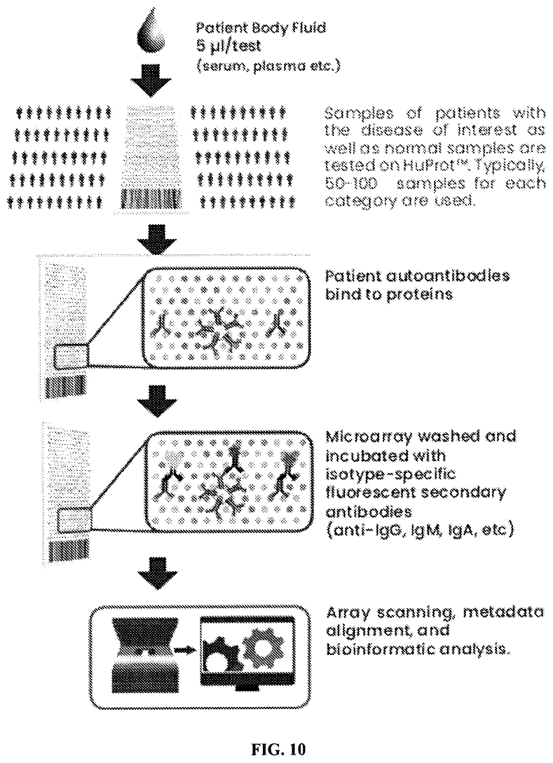

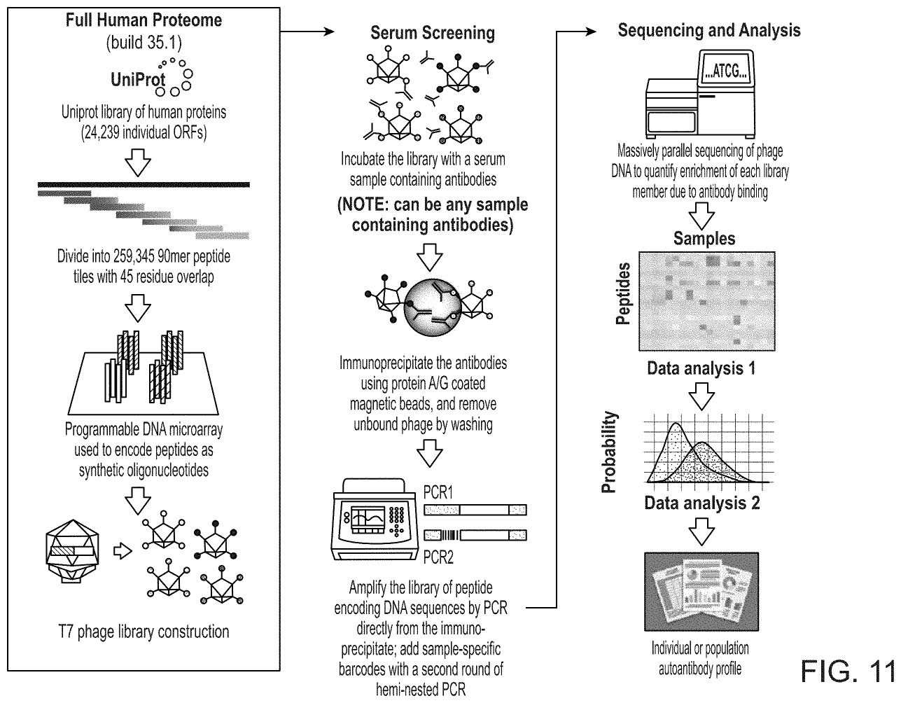

FIG. 10 depicts a schematic outlining a method of using a human proteome microarray for antibody screening. FIG. 11 depicts a schematic outlining a method of using a human proteome microarray for antibody serum screening at an epitope level and subsequent data analysis.

### Example 7: Human Virome Microarray and Usage

A microarray was manufactured presenting the entire UniProt human proteome and UniProt viruses with human tropism as long peptides on bacteriophages. The microarray is limited to IgG detection and cannot provide isotype or subtype-level data. The microarray comprised probes for a peptide library. The peptide library comprises all UniProt viral proteins with human tropism presented as overlapping peptides via a T7 phage display library. The library was reacted with patient antibodies, bound phages were pulled down with protein A/G and sequenced for results. The peptide library comprised 206 species comprising more than 1000 strains in 56-residue peptide tiles with 28-residues overlapping.

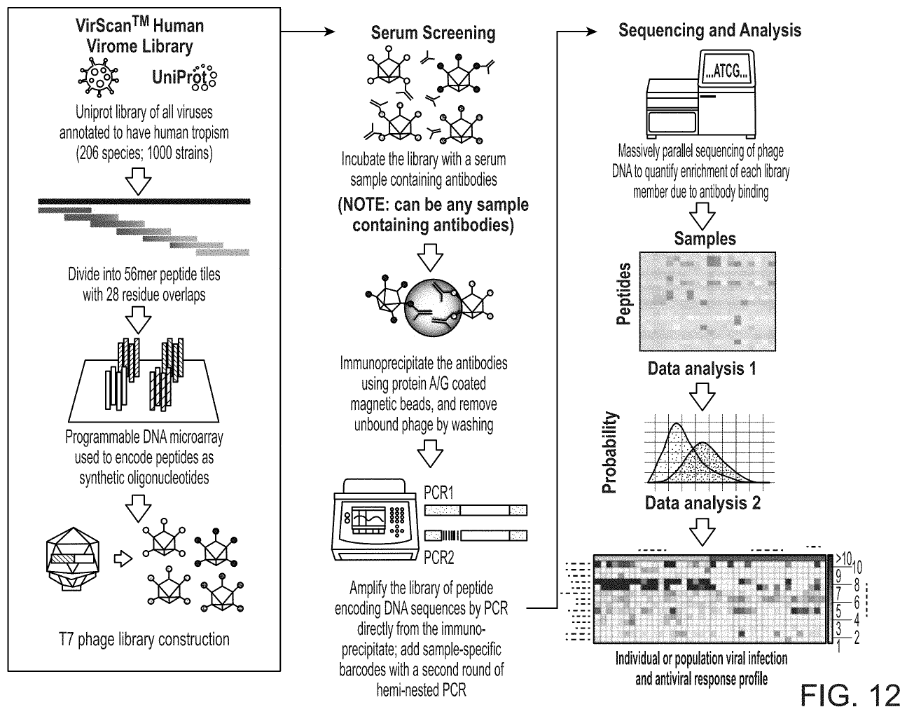

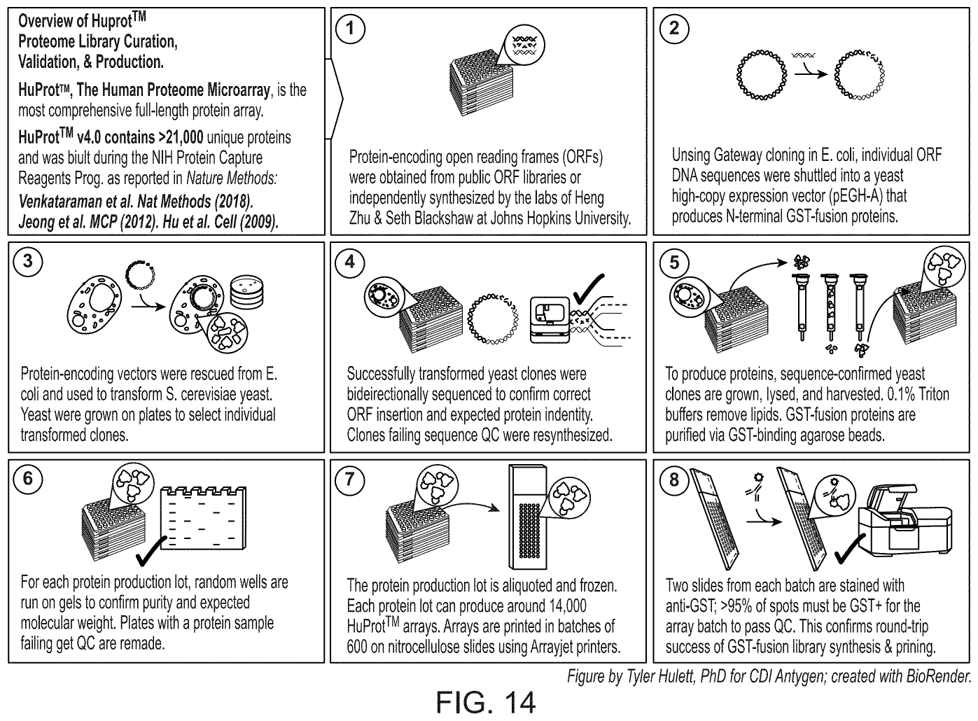

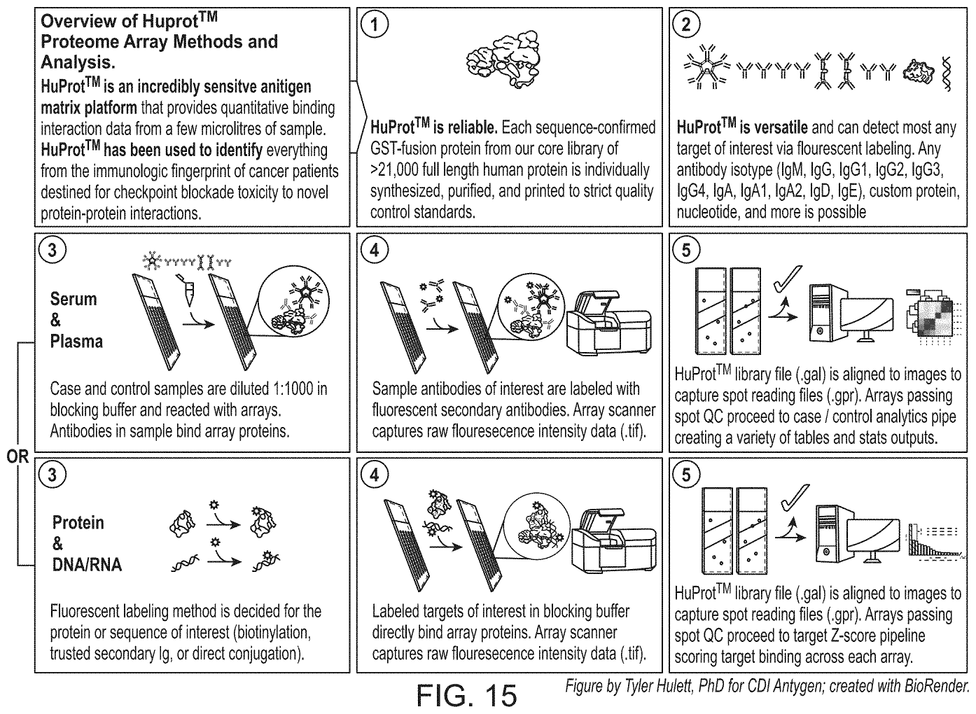

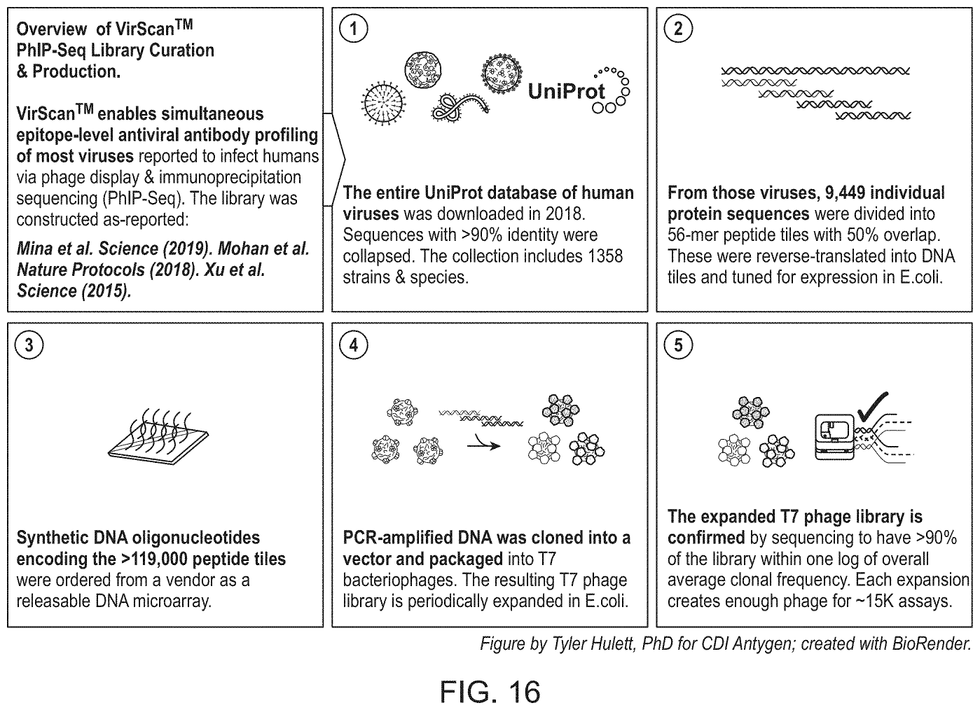

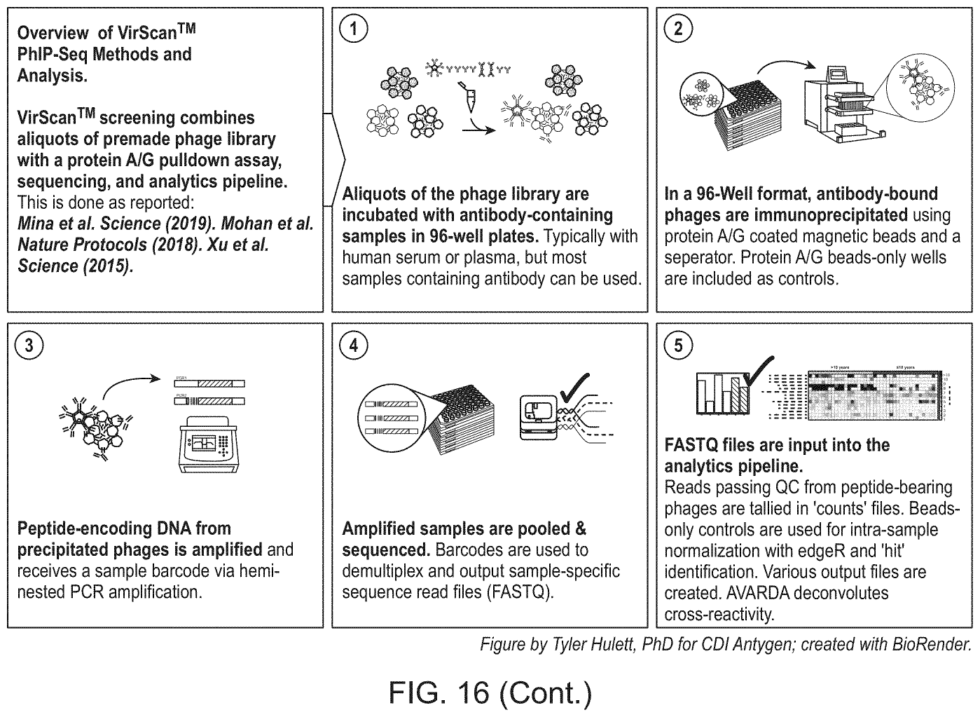

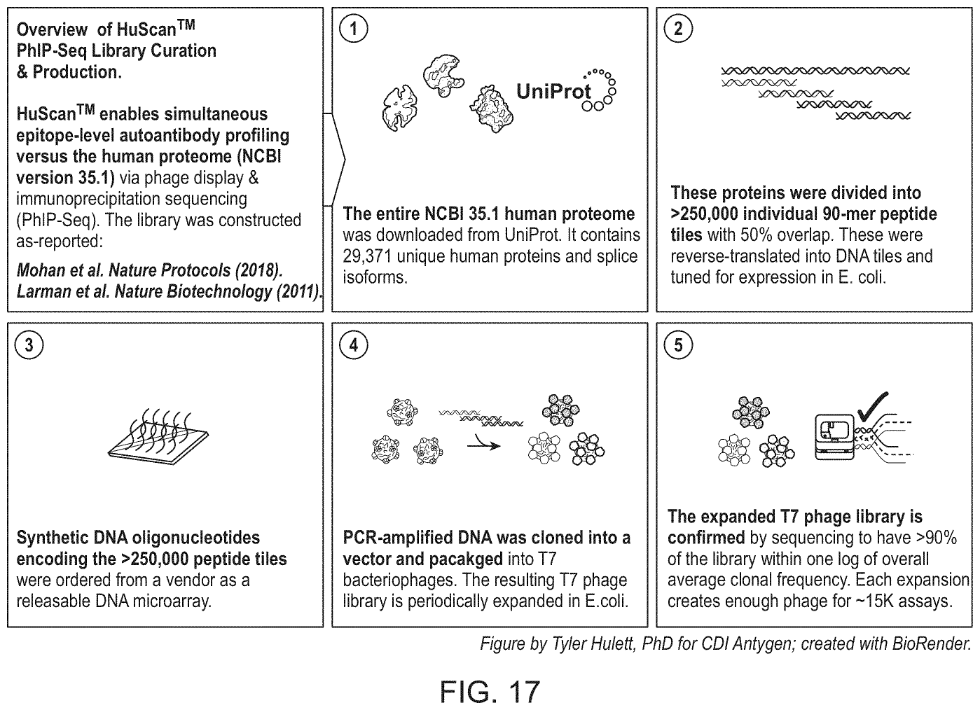

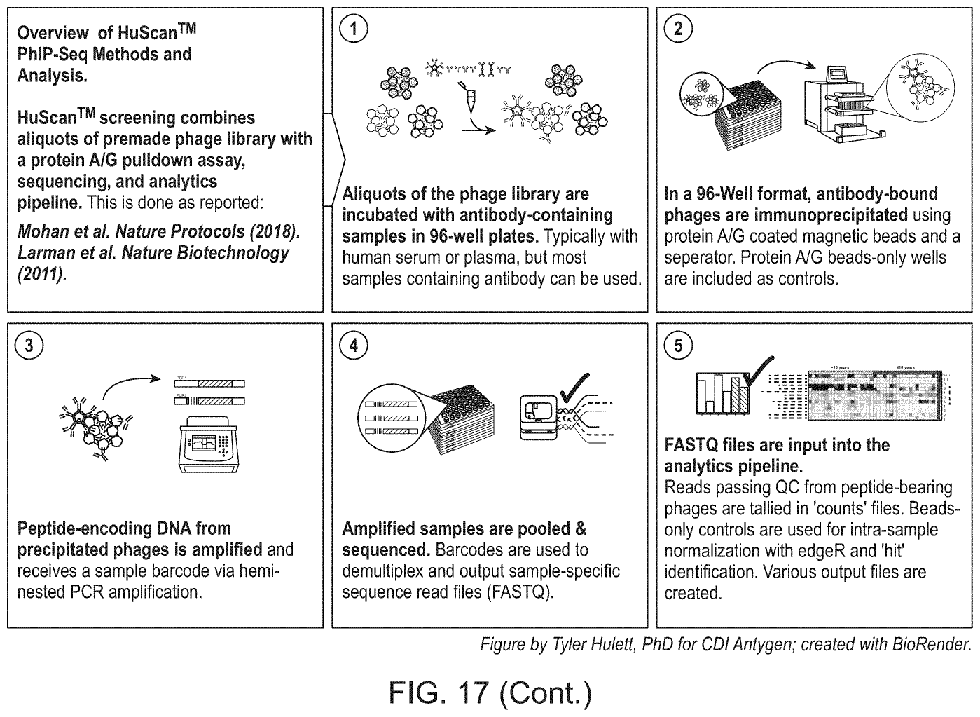

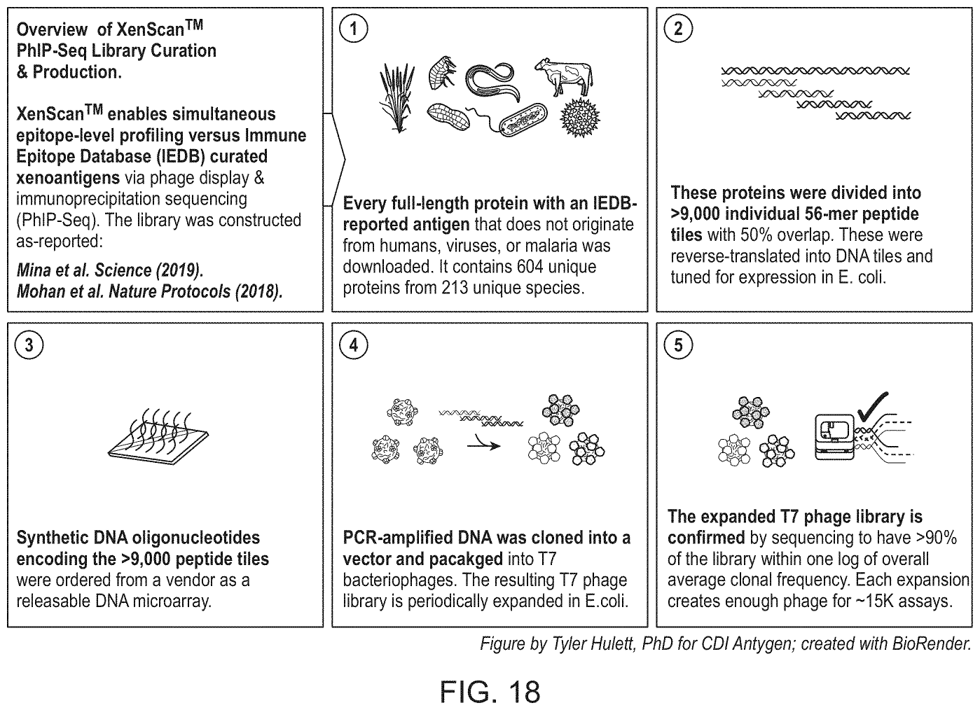

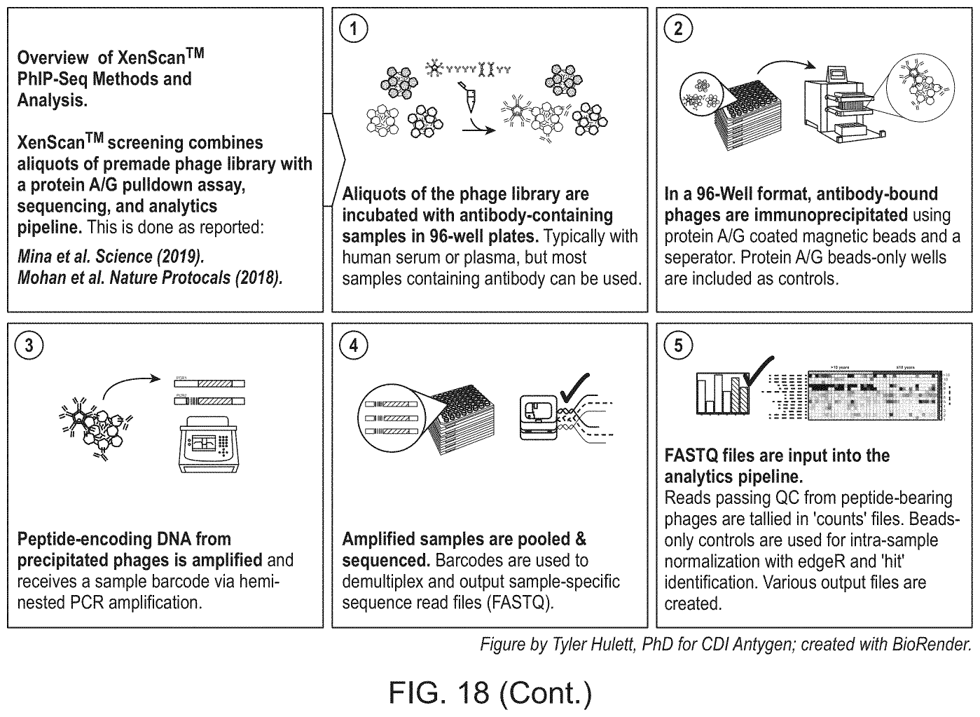

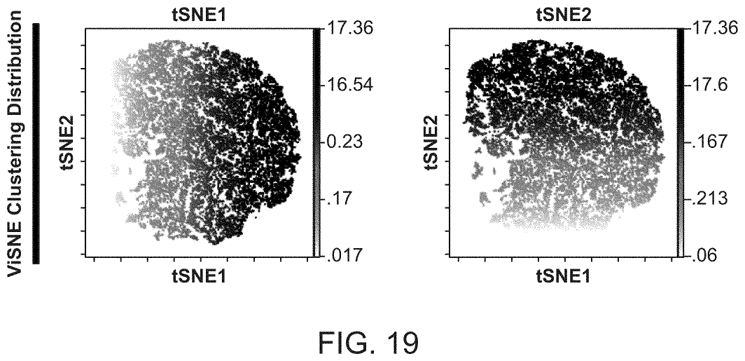

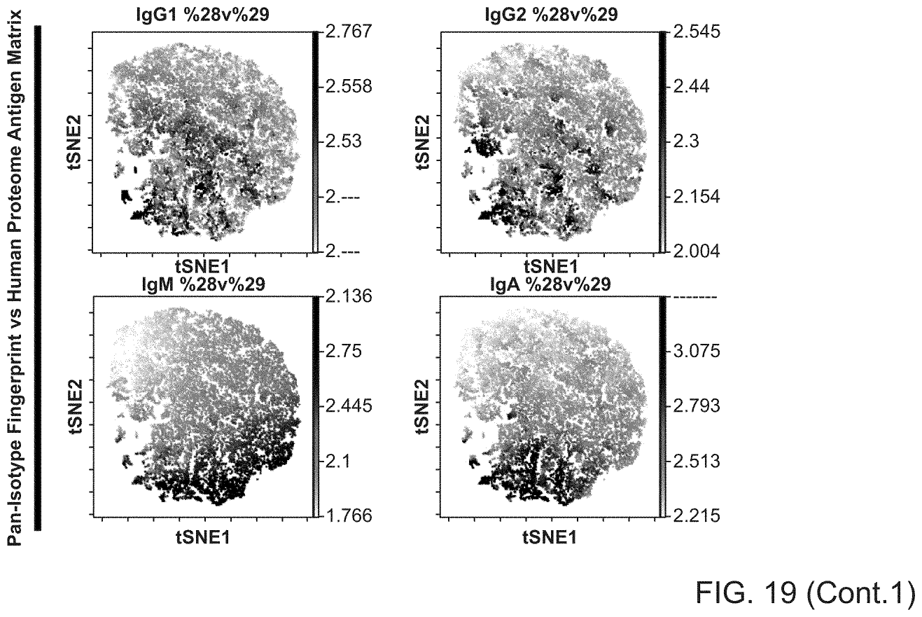

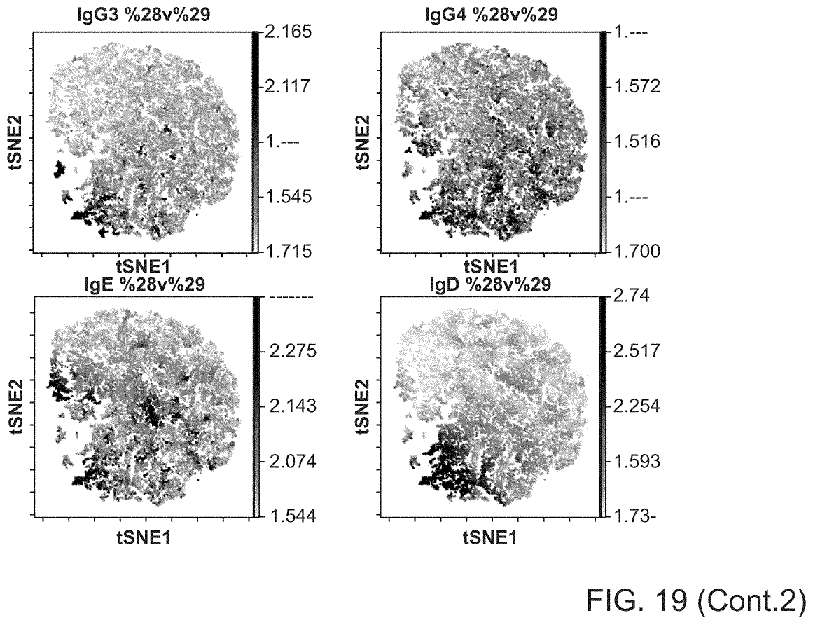

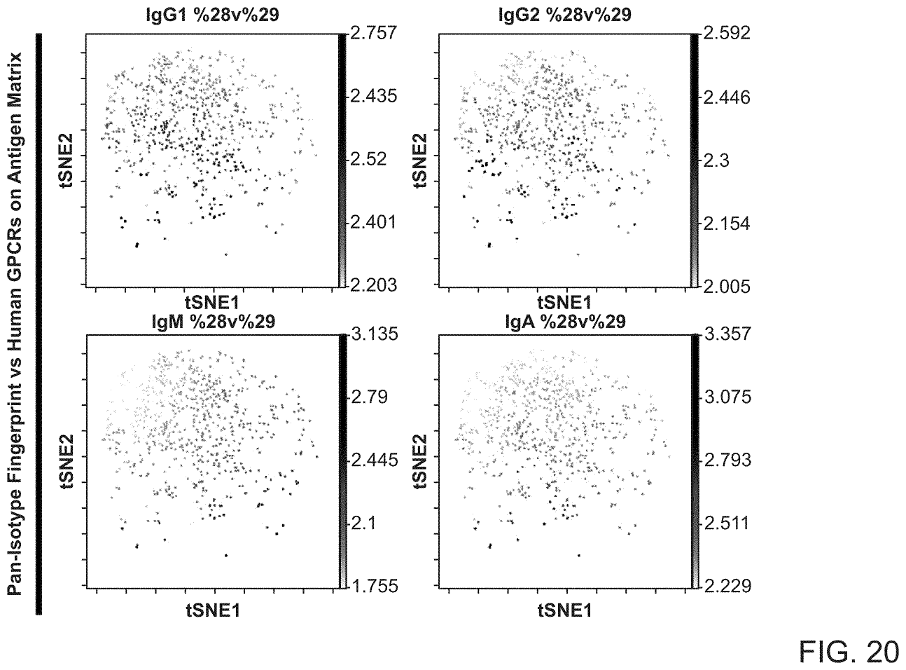

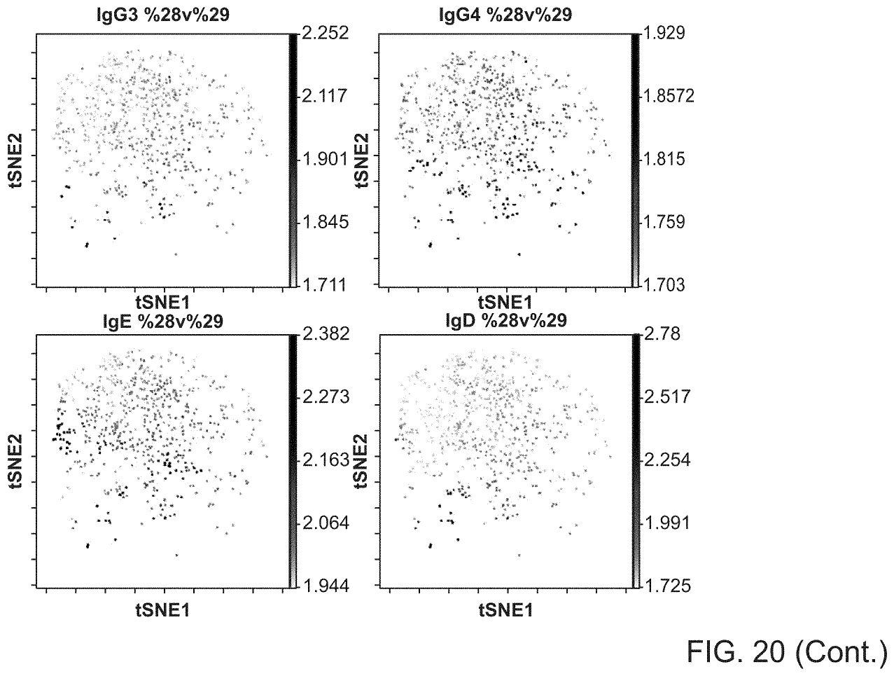

FIG. 12 depicts a schematic outlining a method of using a human virome microarray for antibody serum screening at an epitope level and subsequent data analysis. FIG. 13 shows a computer control system that may be programmed or otherwise configured to implement methods provided herein. FIG. 14 depicts a schematic outlining a method of creating a human proteome microarray for antibody screening. FIG. 15 depicts a schematic outlining a method of using a human proteome microarray for antibody screening. FIG. 16 depicts a schematic outlining a method of using a human virome microarray for antibody serum screening at an epitope level and subsequent data analysis. FIG. 17 depicts a schematic outlining a method of using a human proteome microarray for antibody serum screening at an epitope level and subsequent data analysis. FIG. 18 depicts a schematic outlining a method of using a xenoantigen proteome microarray for antibody serum screening at an epitope level and subsequent data analysis. FIG. 19 shows output from a computer control system that uses machine learning to cluster proteome-wide pan-isotype autoantibody immunoprofiling data from a healthy human male according to multidimensional population-wide antigen-specific interrelationships across immunoglobulin isotypes and subtypes. FIG. 20 shows output from a computer control system that uses machine learning to cluster proteome-wide pan-isotype autoantibody immunoprofiling data from a healthy human male according to multidimensional population-wide antigen-specific interrelationships across immunoglobulin isotypes and subtypes filtered to demonstrate this output associates with gene ontology categories of interest—in this case druggable GPCRs.

### Example 8: Pan-Isotype Immunoglobulin Fingerprinting by Human Proteome Antigen Matrix Demonstrates Population-Wide Antigen-Specific Multiplex Interrelationships Across Immunoglobulin Isotypes and Subtypes that Extend to Pharmacologically Relevant GPCRs

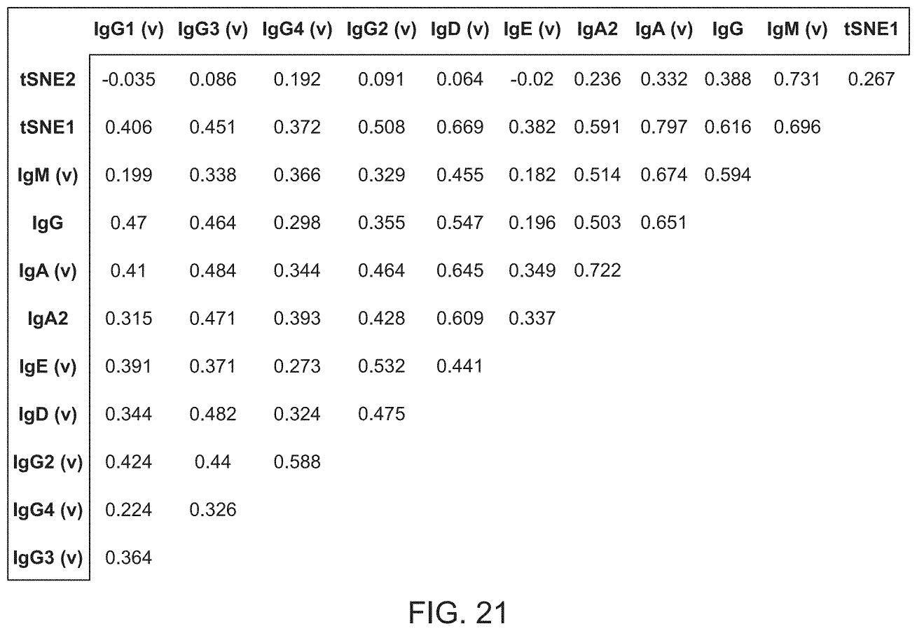

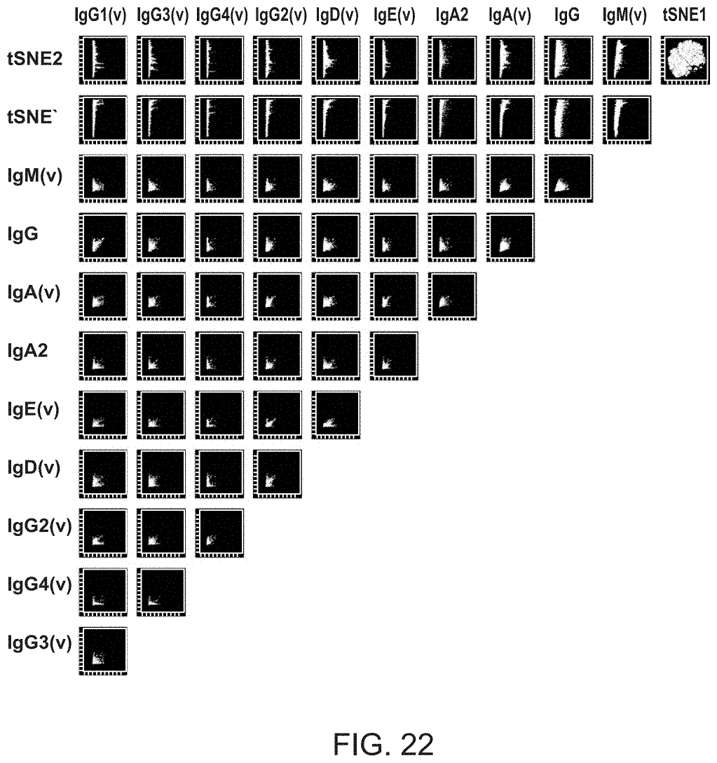

Serum was collected from a healthy adult human male donor, incubated on HuProt human proteome antigen matrices containing >21,000 individual human proteins and stained with pairs of anti-isotype secondaries on four individual slides. The full eight channel analysis was scanned and log 2 transformed. Matrix data were filtered to remove positive controls and immunoglobulins. Matrix library overlap with the Human Protein Atlas was used to identify GPCRs. Data were uploaded to Cyto-bank and viSNE clustering was performed across the cohort to compute multiparameter population interrelationships for all displayed channels (IgG1, IgG2, IgG3, IgG4, IgM, IgA, IgE, IgD) as shown in FIG. 19 and FIG. 20. While the autoantibody data appears to be more continuous than single-cell staining data viSNE was designed for, there still appear global gradients and multiplex population islands within the data structure. In particular, IgM appears to create a large gradient structure of autoantibody binding across the human proteome. When data were filtered to display only the 721 GPCRs within the matrix, similar patterns were observed such as IgM avoidance of GPCR binding—potentially due to natural tolerance mechanisms in this individual. Correlation coefficients of each individual isotype ratio pair included in the analysis demonstrated some isotypes were more closely linked to one another than others (FIG. 21). Individual plots of log 10-transformed isotype-pair comparisons used to compute above correlation coefficients are shown in FIG. 22.

### Example 9: Pan-Isotype Serum Autoantibody Fingerprints

To reduce to practice pan-isotype serum autoantibody fingerprints from selected exemplary healthy and disease donors, we utilized a human proteome microarray (HuProt Human Proteome Microarray v4.0, CDI Laboratories, Mayaguez, PR) that contains over 21,000 unique, individually-purified full-length human proteins in duplicate, covering more than 81% of the proteome [ref: Jeong J S, Jiang L, Albino E, et al. Rapid identification of monospecific monoclonal antibodies using a human proteome microarray. Mol Cell Proteomics. 2012; 11(6):0111.016253. doi:10.1074/mcp.0111.016253]. Multiple arrays were utilized for each patient in order to cover all possible antibody isotypes and subtypes. Briefly, the HuProt arrays were blocked with blocking solution (5% BSA/1×TBS-T) at room temperature for 1 h, and then probed with serum samples (diluted 1:1000) at 4° C. overnight. The arrays were then washed with 1×TBS-T for 3 times, 10 min each, and probed with up to two anti-human IgG isotype and subtype-specific secondary antibodies (anti-IgG, anti-IgG1, anti-IgG2, anti-IgG3, anti-IgG4, anti-IgA, anti-IgA1, anti-IgA2, anti-IgM, anti-IgD, and anti-IgE) at room temperature for 1 h, followed by three washes of 1×TBS-T, 10 min each, and then dried with an air duster and scanned using a GenePix 4000B instrument (Molecular Dynamics, Sunnyvale, Calif.). GenePix Pro (v7.2.30) software was used to measure the signal intensities for immunoglobulin binding to array features as well as any background signal present. Array signal intensities data were quality controlled for successful printing, staining, and scanner alignment using internal software tools (CDI Laboratories, Mayaguez, PR) which confirmed duplicate spots retained >0.95 R² signal intensity correlation across each array included in this study. Raw data from replicate spot pairs were averaged, log 2 transformed, and grouped to create individual pan-isotype .csv files for each patient. These were submitted to flow cytometry analysis software (FlowJo) for multi-isotype gating, plotting, and analysis. In the case of our lupus case, we followed gating patterns which associated with the known lupus antigens Sm/RNP Complex, Sm antigen, and SNRPB into deeper multi-isotype autoantigen subpopulations. We performed gene ontology enrichment analysis on the resulting autoantigen subpopulations using Enrichr.

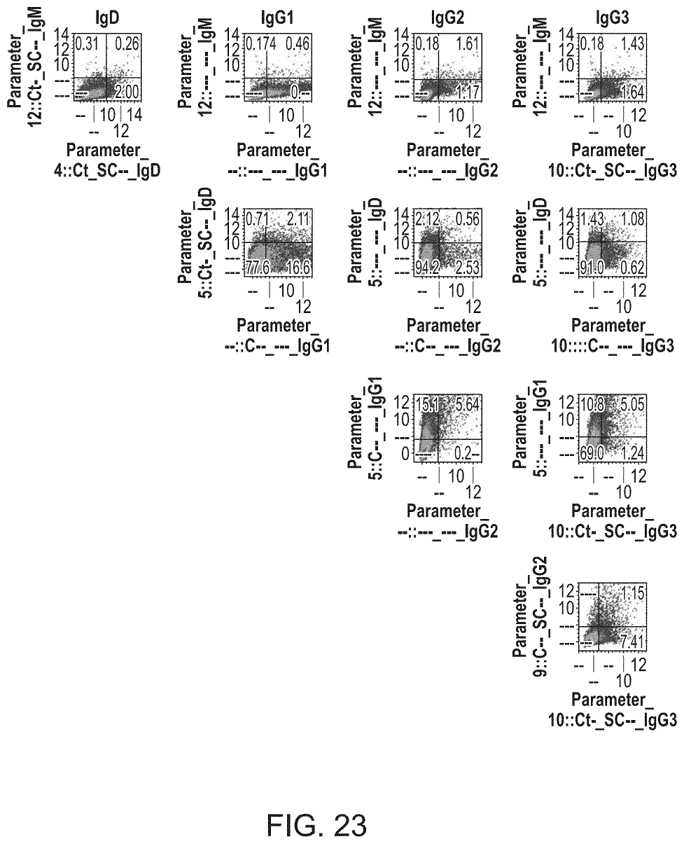

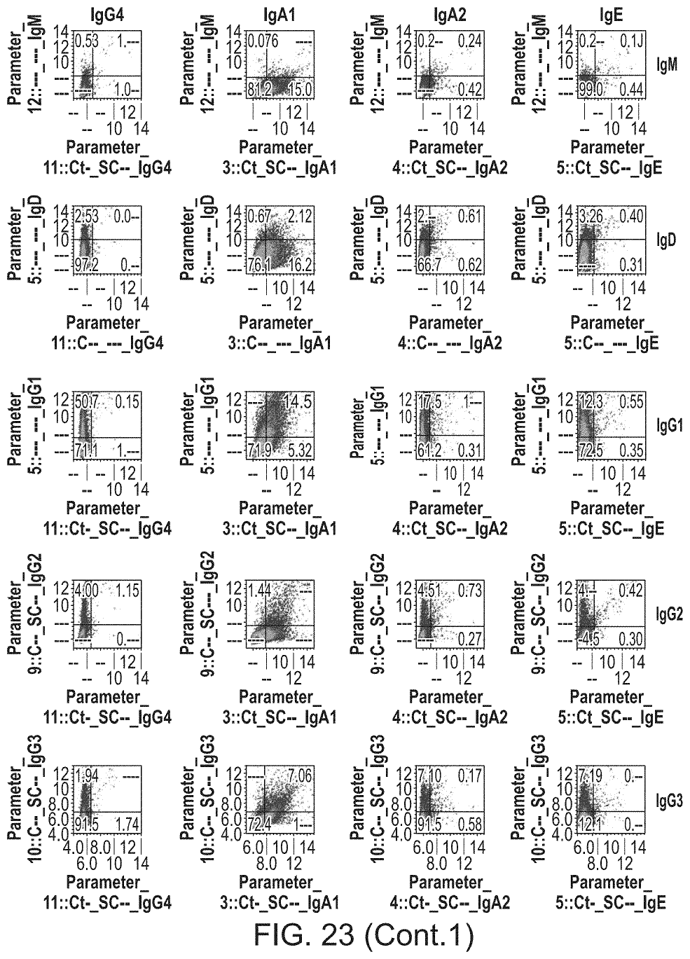

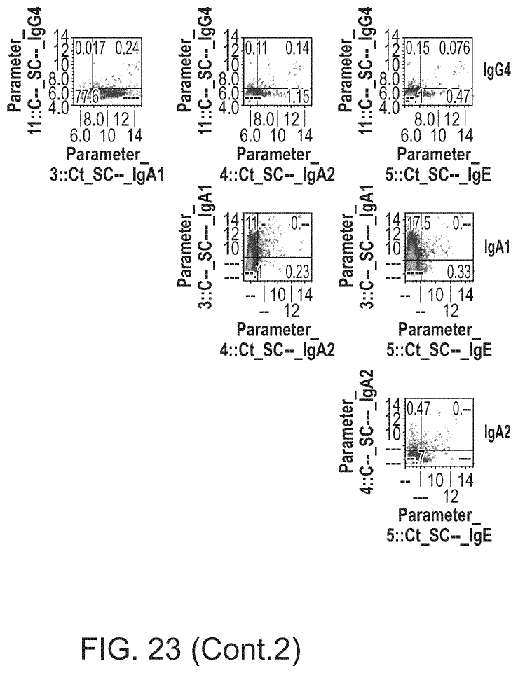

FIG. 23 depicts raw log 2 transformed proteome-wide pan-isotype autoantibody immunoprofiling of a child with Systemic lupus erythematosus (SLE). The data demonstrates unique population-wide antigen-specific interrelationships across immunoglobulin isotypes and subtypes associated with lupus.

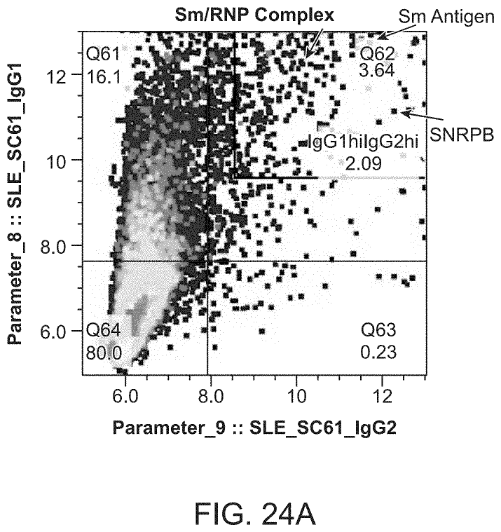

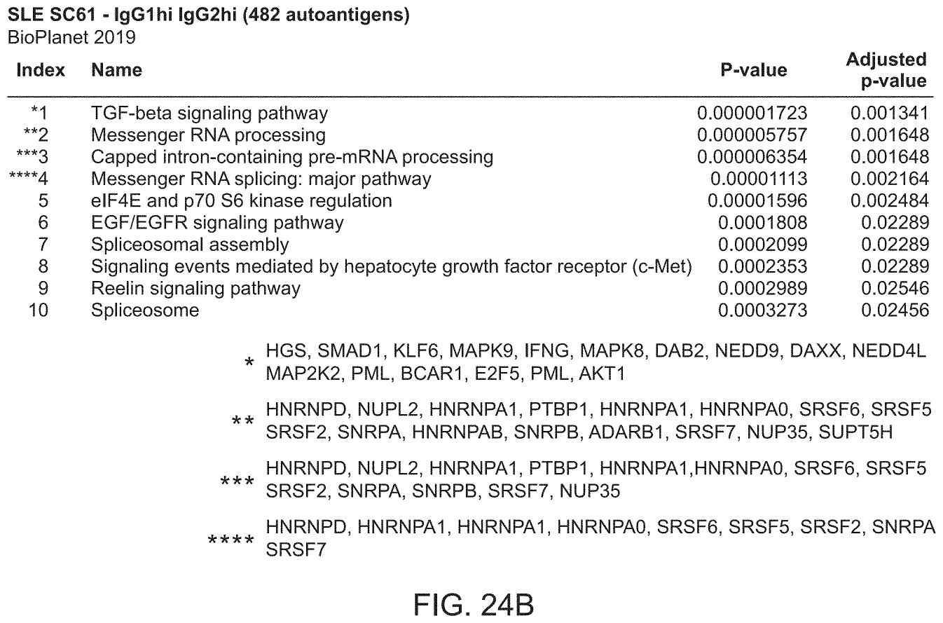

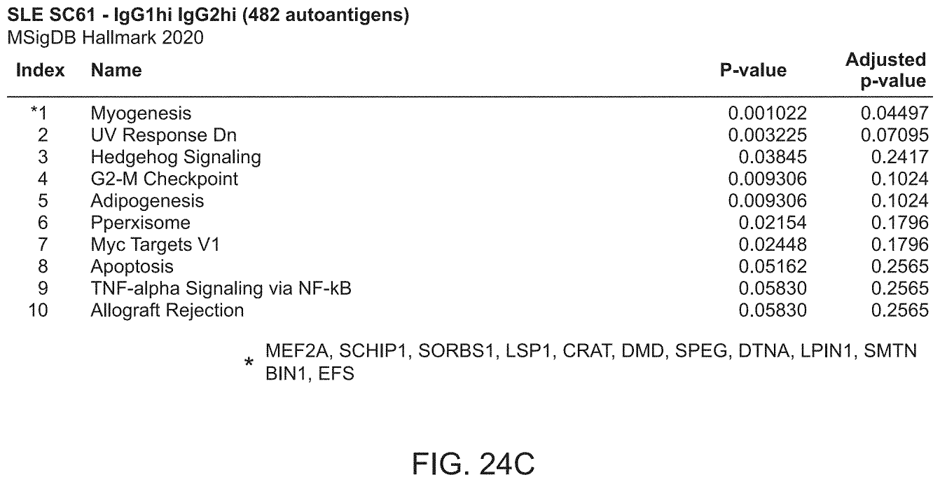

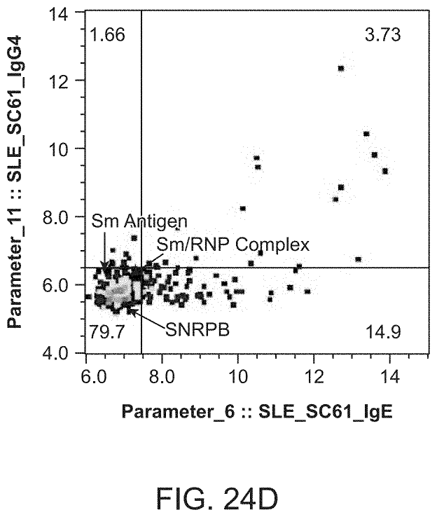

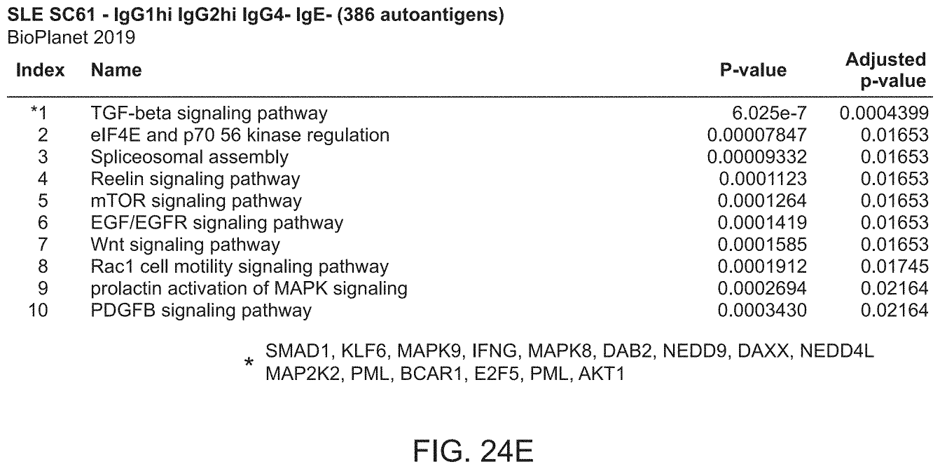

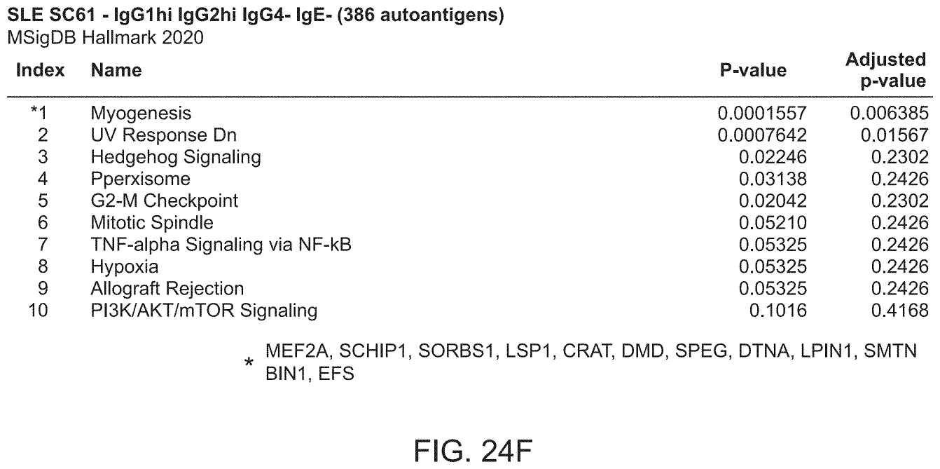

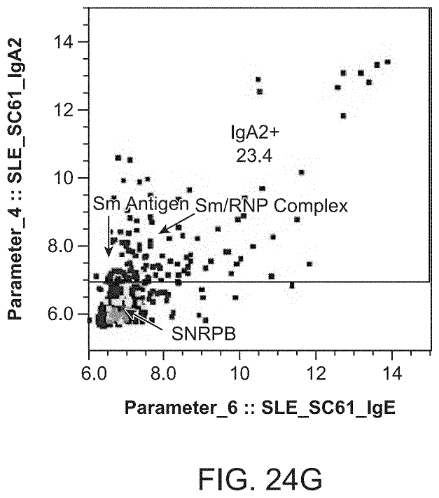

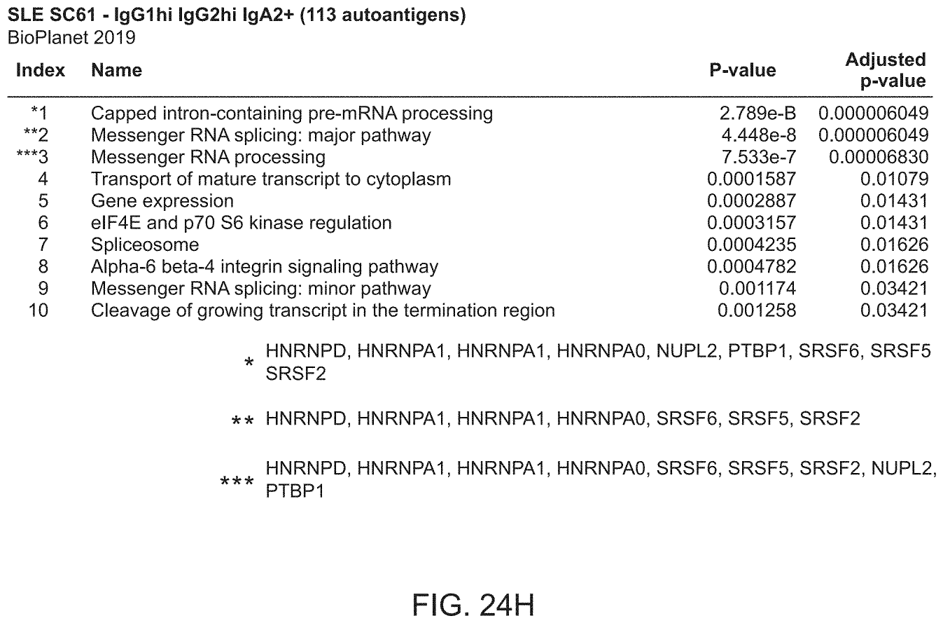

FIG. 24 depicts the utility of this invention via raw log 2 transformed proteome-wide pan-isotype autoantibody immunoprofiling of a child with Systemic lupus erythematosus (SLE). The known lupus autoantigens Sm/RNP Complex, SM Antigen, and SNRPB are contained in the ‘IgG1high IgG2high’ autoantibody staining compartment. The autoantigens in this compartment are significantly enriched by gene ontology enrichment analysis for antigens from TGF-beta signaling cytokines and molecules, messenger RNA processing, capped intron-containing pre-mRNA processing, messenger RNA splicing, major pathway, and myogenesis; pathways known to behave aberrantly in lupus. When filtering for additional isotypes, these enrichments isolate to specific multi-isotype sub-compartments. TGF-beta signaling cytokines and molecules and myogenesis-associated biomarkers are contained within the ‘IgG1high IgG2high IgG4negative IgEnegative’ sub-compartment. The mRNA associated messenger RNA processing, capped intron-containing pre-mRNA processing, and messenger RNA splicing, major pathway biomarkers are contained within the ‘IgG1high IgG2high IgGA2positive’ sub-compartment.

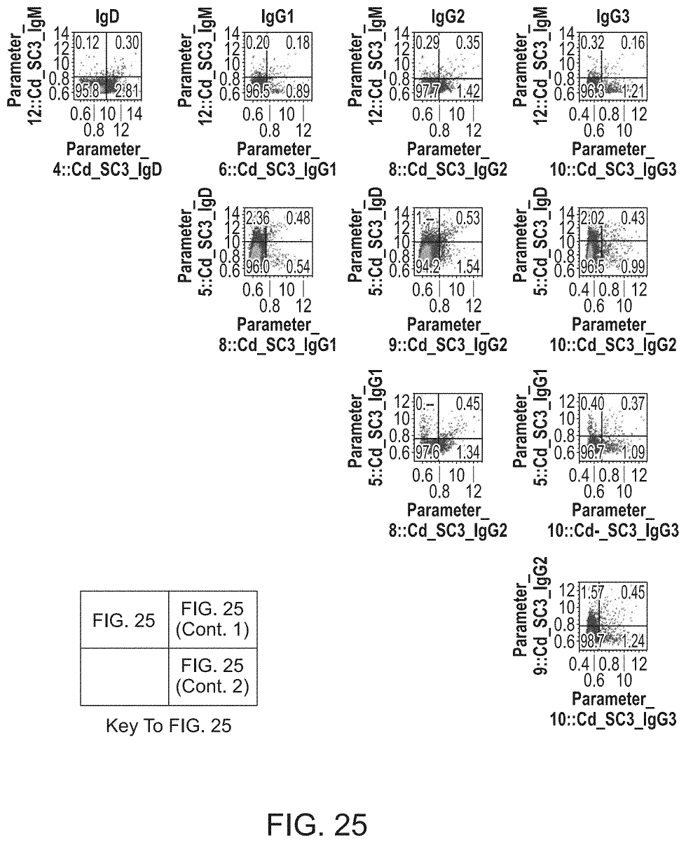

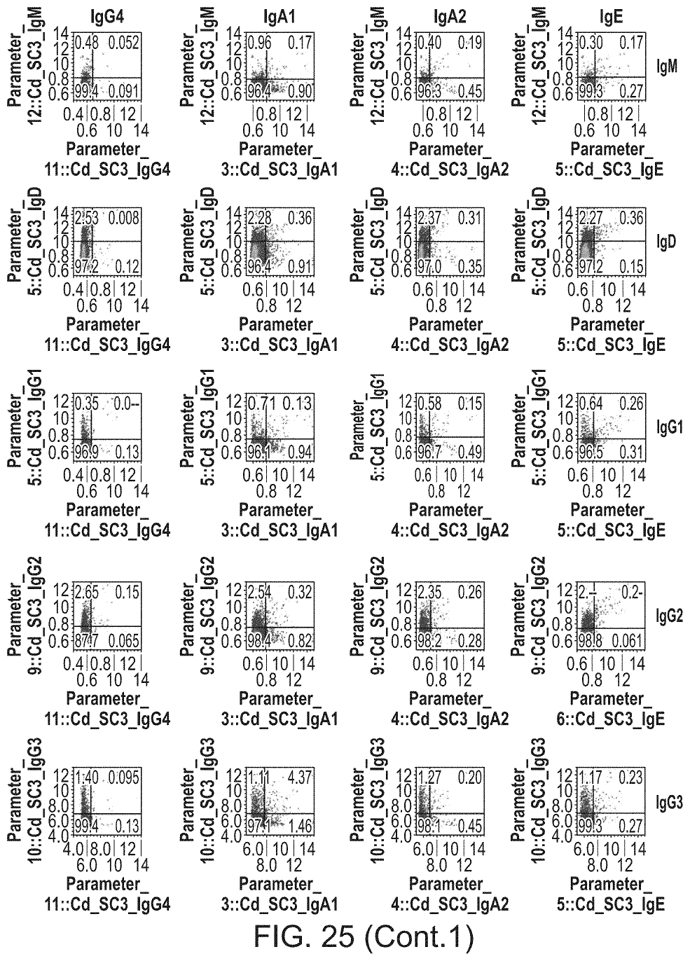

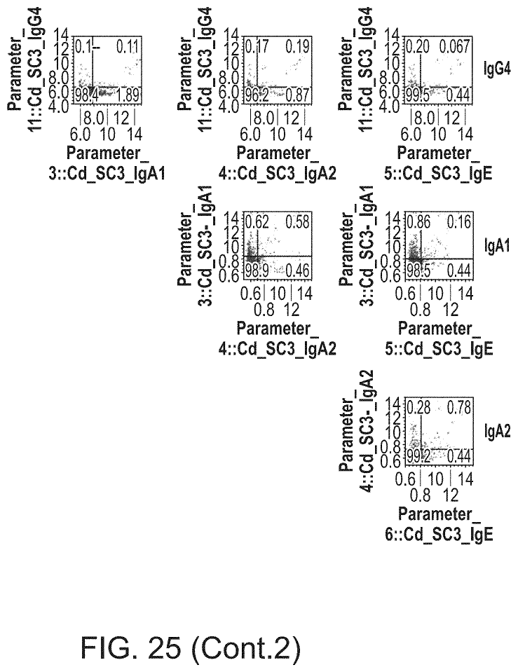

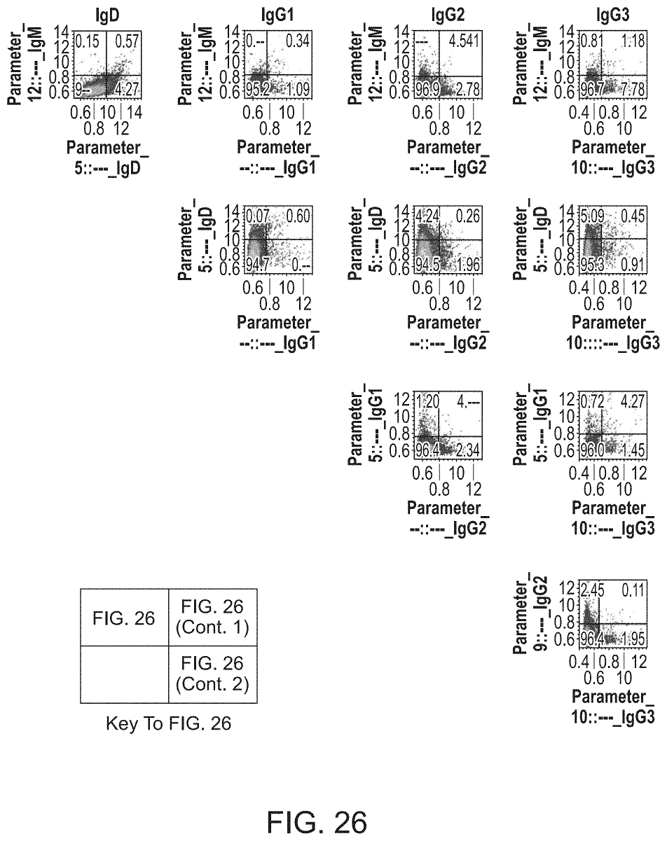

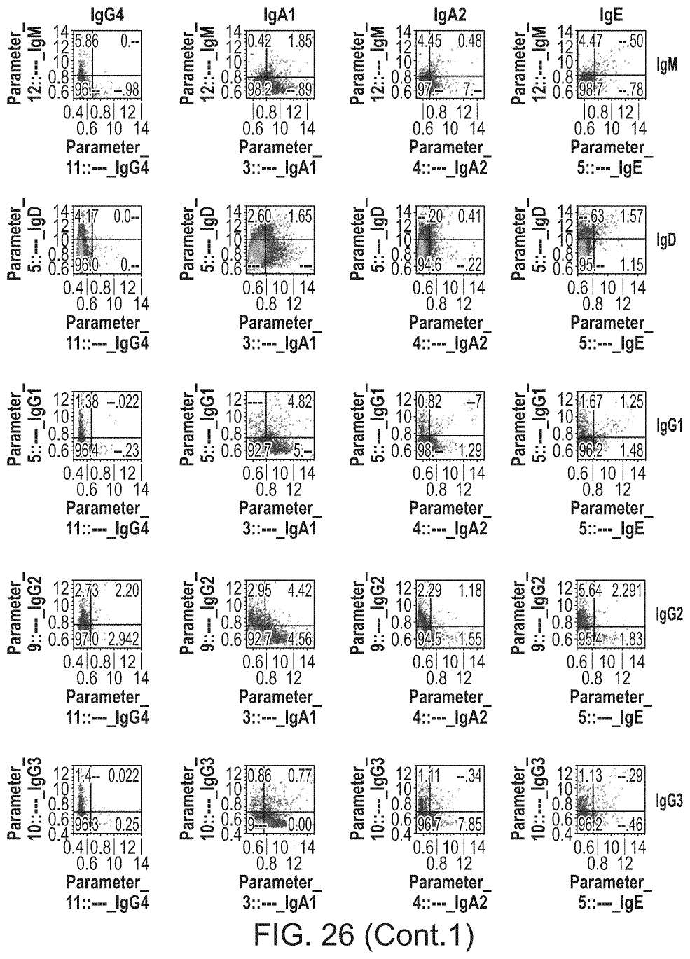

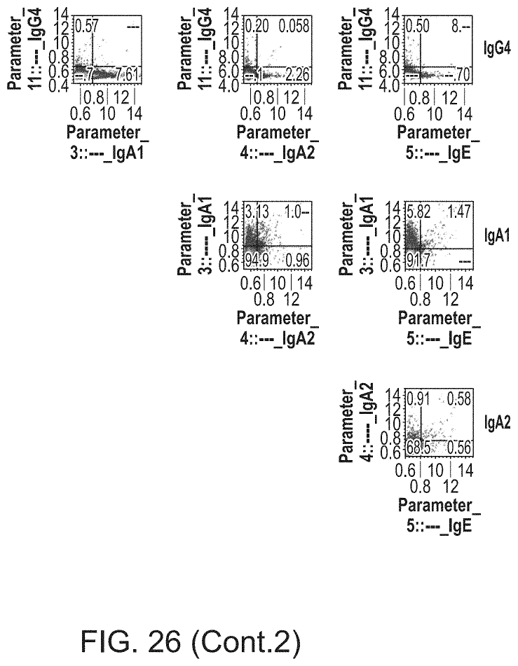

FIG. 25 and FIG. 26 depict raw log 2 transformed proteome-wide pan-isotype autoantibody immunoprofiling of a healthy child. The data demonstrates unique population-wide antigen-specific interrelationships across immunoglobulin isotypes and subtypes.

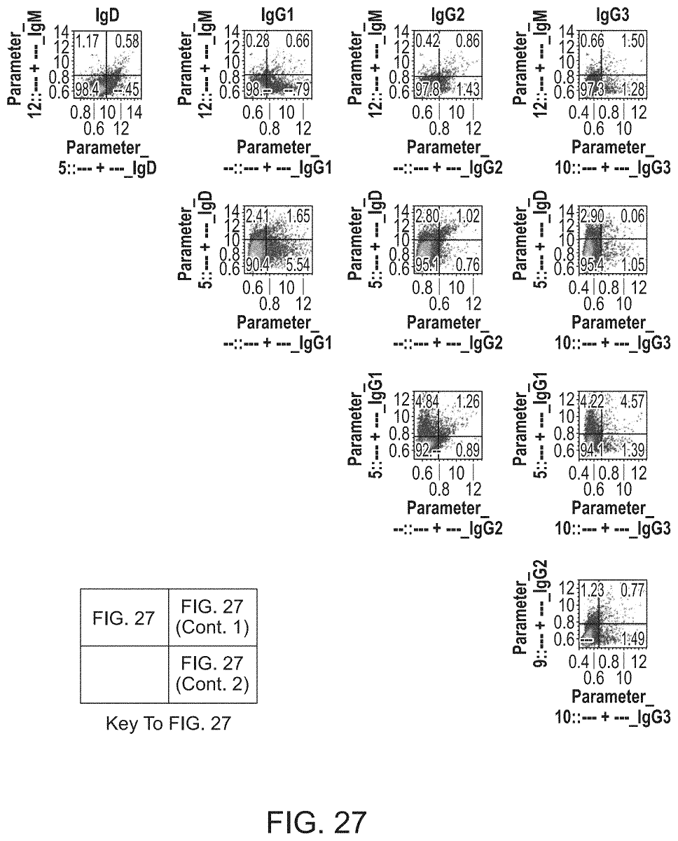

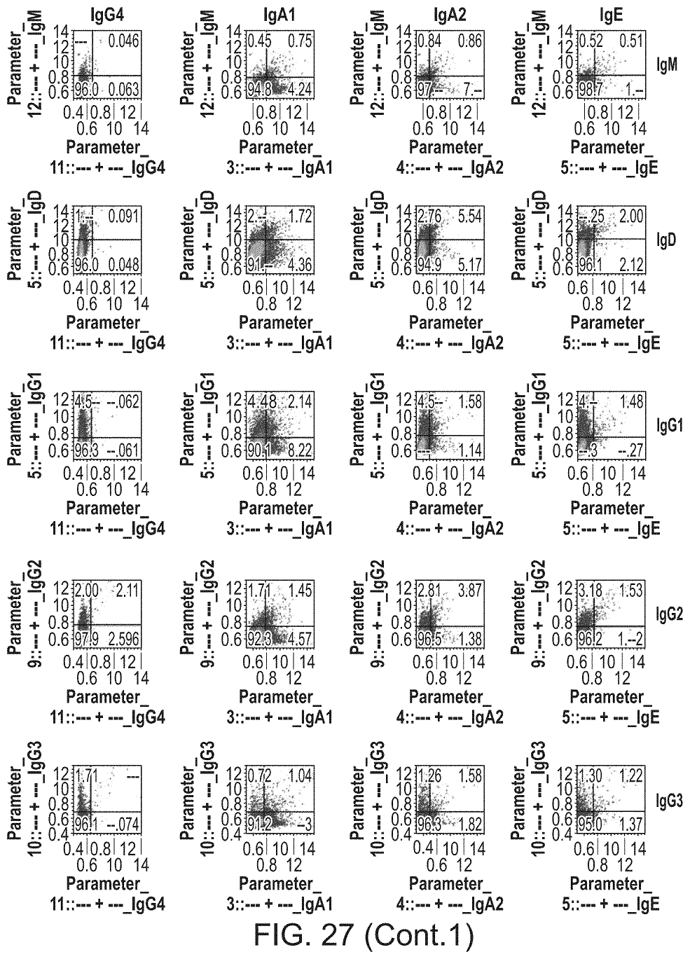

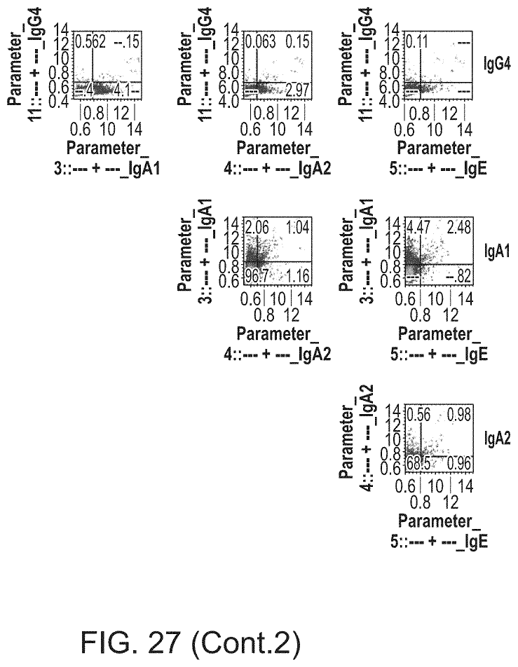

FIG. 27 depicts raw log 2 transformed proteome-wide pan-isotype autoantibody immunoprofiling of the previous healthy adult, one year later and recently recovered from viral shingles and COVID-19 vaccination. The data demonstrates unique population-wide antigen-specific interrelationships across immunoglobulin isotypes and subtypes that either change or are stable with time and are associated with viral shingles and COVID-19 vaccination.

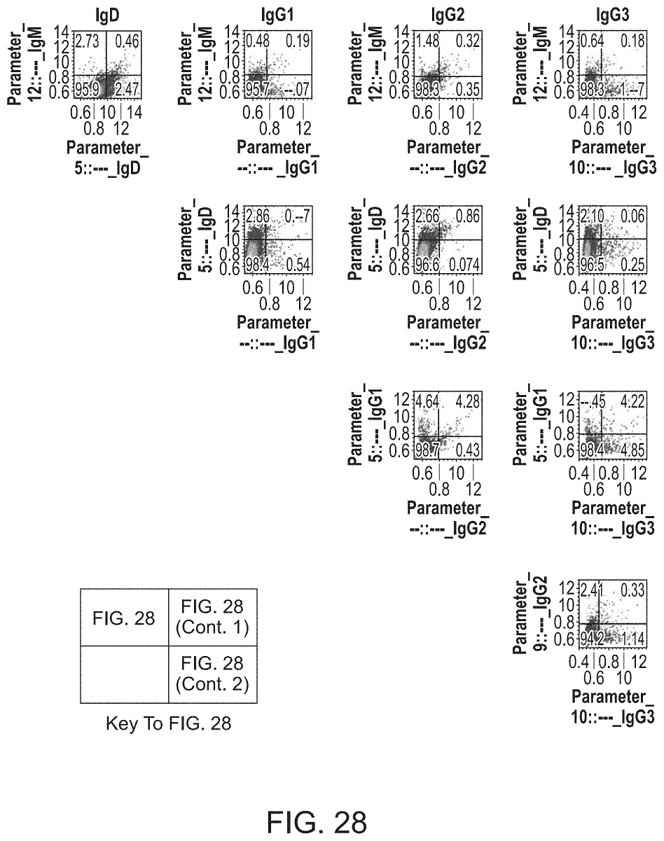

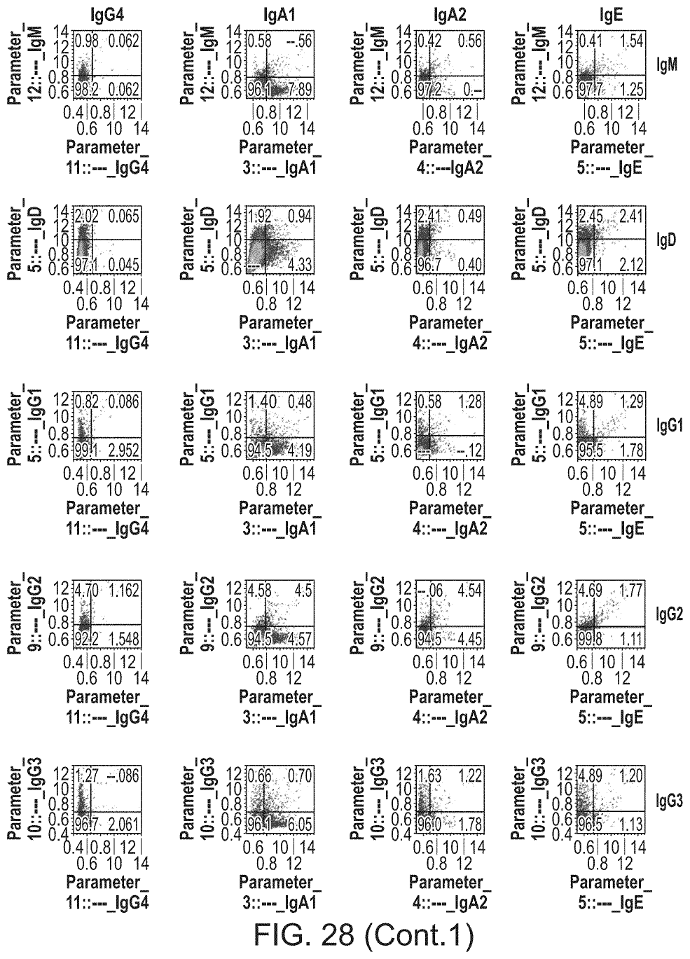

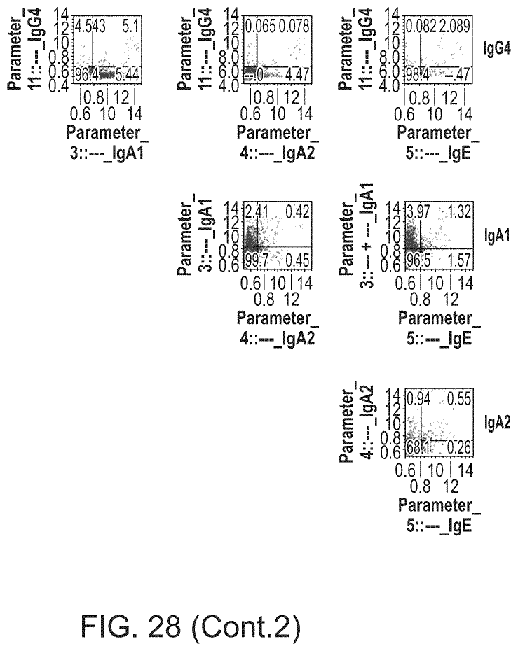

FIG. 28 depicts raw log 2 transformed proteome-wide pan-isotype autoantibody immunoprofiling of a child with COVID-19-induced MISC autoimmune disease. The data demonstrates unique population-wide antigen-specific interrelationships across immunoglobulin isotypes and subtypes associated with COVID-19-induced MISC autoimmune disease. TOLLIP deficiency has been previously reported as a causative agent of ulcerative colitis in mouse models. However—human colonic epithelium from inflammatory bowel disease patients has been reported to be at normal levels (Steenholdt et al., (2009) Expression and function of toll-like receptor 8 and Tollip in colonic epithelial cells from patients with inflammatory bowel disease, Scandinavian Journal of Gastroenterology, 44:2, 195-204, DOI: 10.1080/00365520802495529). Normal levels of colonic TOLLIP—blocked by IgA autoantibodies in the colonic mucosa—would be a way of reconciling these two studies. A deficiency of TOLLIP function could be created by anti-TOLLIP function-blocking autoantibodies in the presence of normal levels of TOLLIP protein. While TOLLIP function is primarily believed to be intracellular, there is abundant evidence for TOLLIP protein in serum and the extracellular space. We propose that TOLLIP is a natural tonic immune signal, sometimes disrupted in the colon by anti-TOLLIP IgA autoantibodies, resulting in the disease ulcerative colitis. To support this theory of TOLLIP as natural immune checkpoint—we investigated TOLLIP expression in patients from The Cancer Genome Atlas (TCGA; accessed via https://www.cbioportal.org/). We first compared the most checkpoint-blockade-susceptible of all tumors—malignant melanoma. Approximately 5% of malignant melanoma patients in this cohort overexpressed TOLLIP mRNA, and their overall survival was significantly worse. This supports the hypothesis that TOLLIP is a natural immune checkpoint, overexpressed by melanoma to protect itself from the adaptive immune system. These data support anti-TOLLIP therapies (monoclonal antibody, DART, BiTE, etc) being a novel checkpoint blockade treatment for malignant melanoma. Additionally, we observed similar results in glioma brain cancer, supporting a similar treatment for other types of cancer.

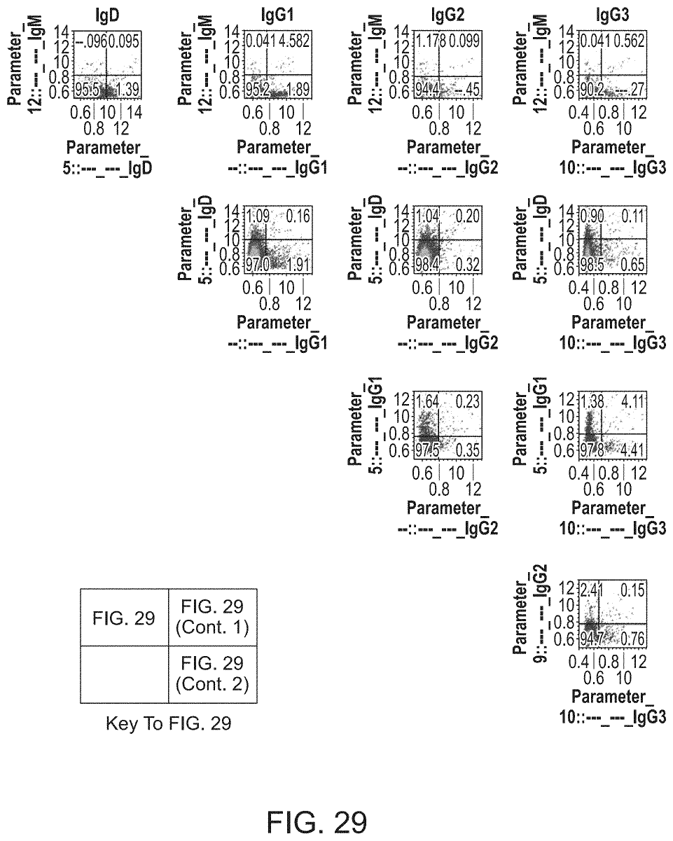

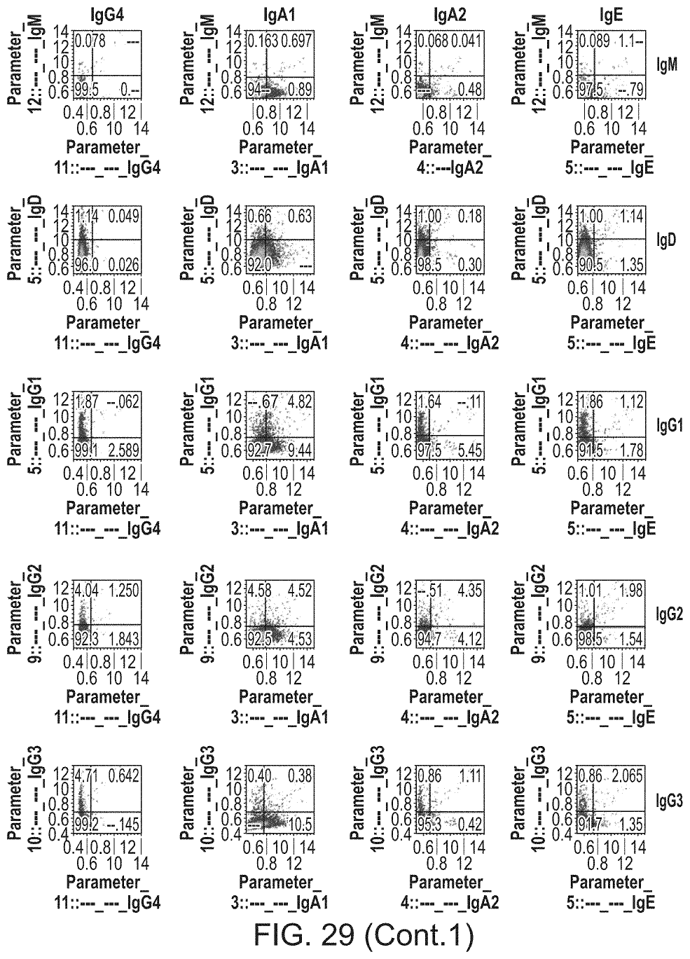

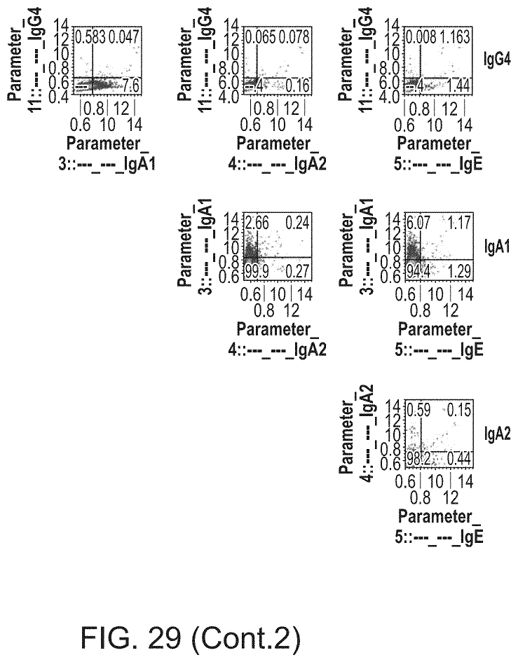

FIG. 29 depicts raw log 2 transformed proteome-wide pan-isotype autoantibody immunoprofiling of a patient with CTLA-4-knockout genetically inherited immune disease. The data demonstrates unique population-wide antigen-specific interrelationships across immunoglobulin isotypes and subtypes associated with CTLA-4-knockout genetically inherited immune disease.

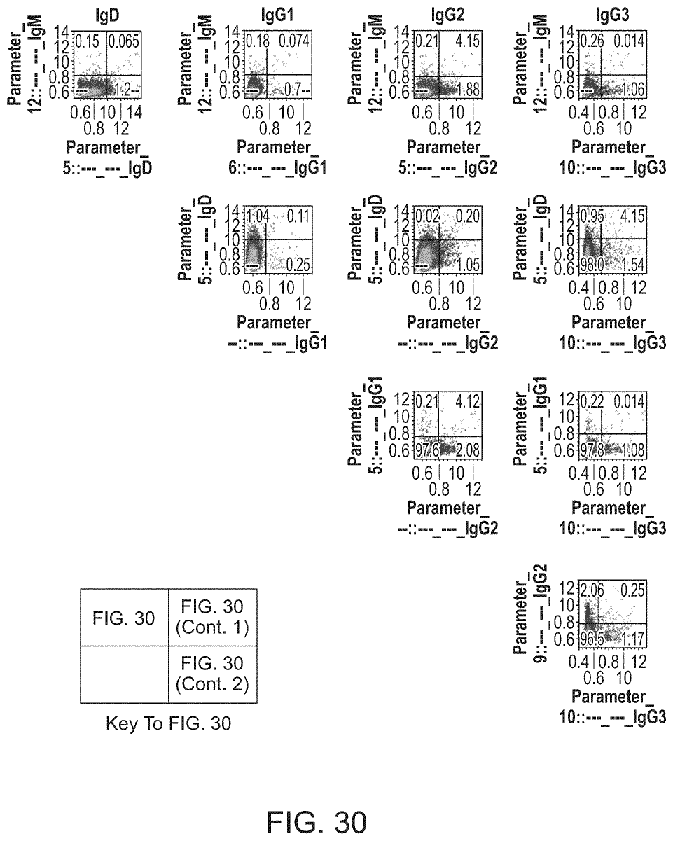

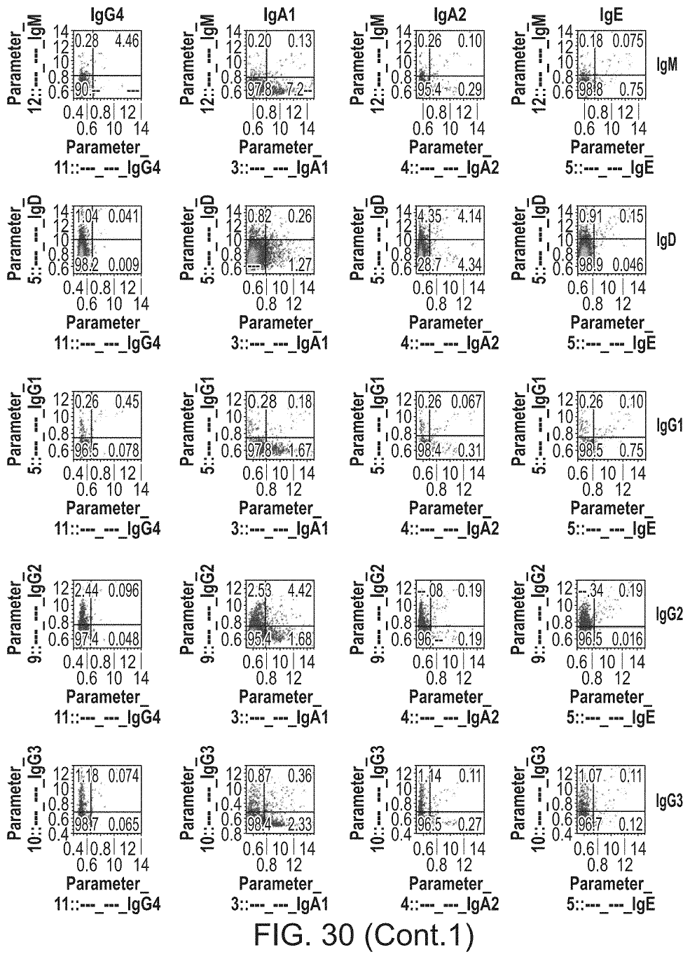

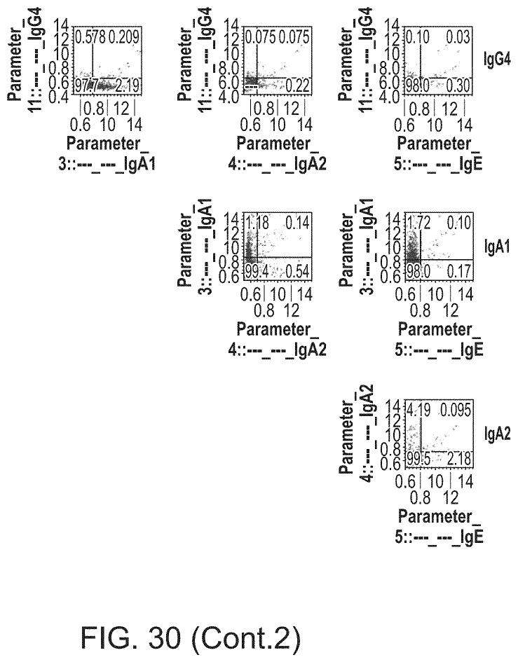

FIG. 30 depicts raw log 2 transformed proteome-wide pan-isotype autoantibody immunoprofiling of a patient with cancerous lymphoma. The data demonstrates unique population-wide antigen-specific interrelationships across immunoglobulin isotypes and subtypes associated with cancerous lymphoma.

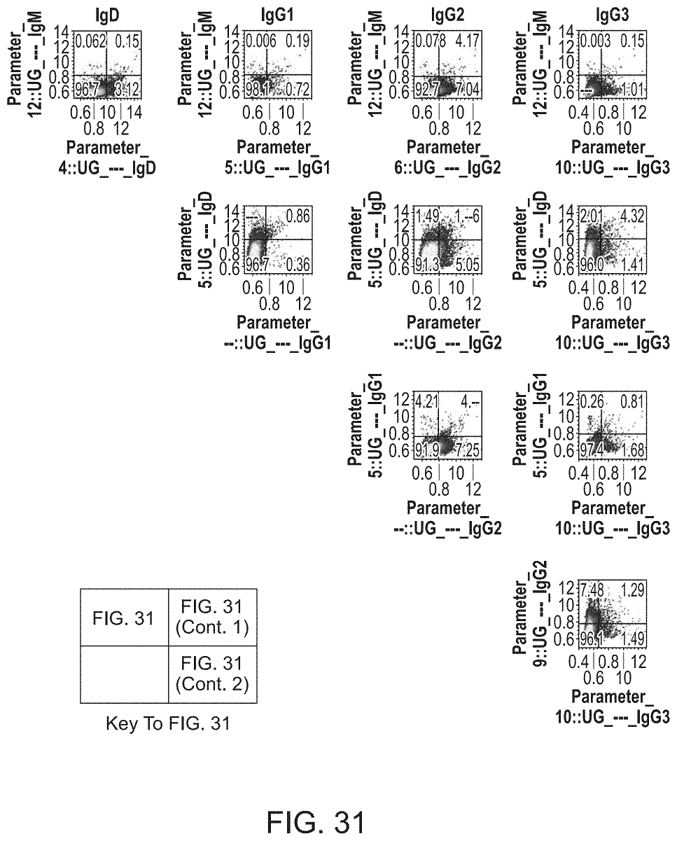

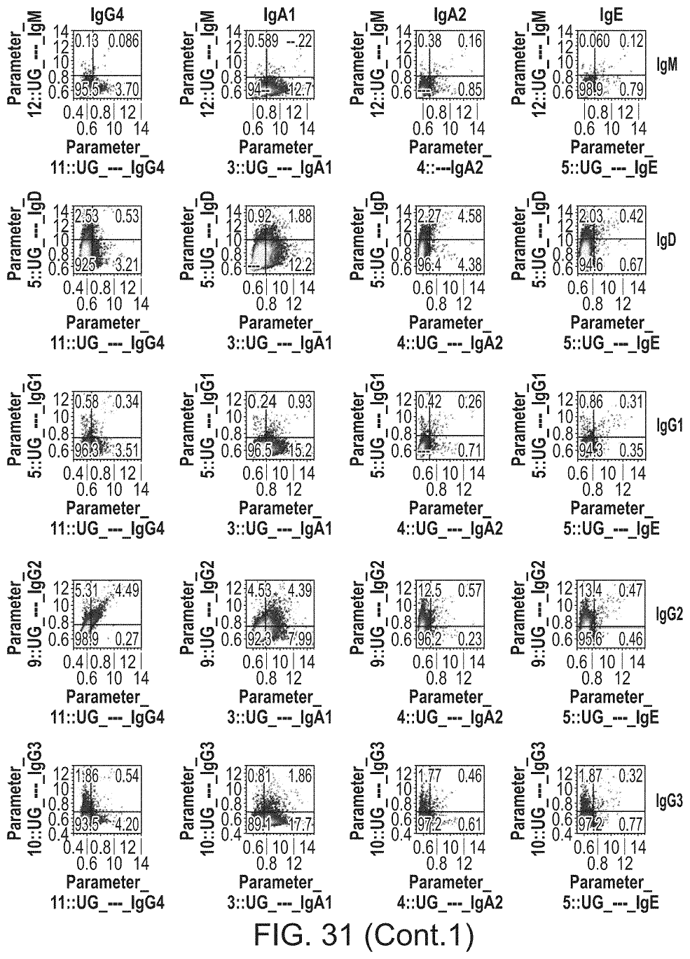

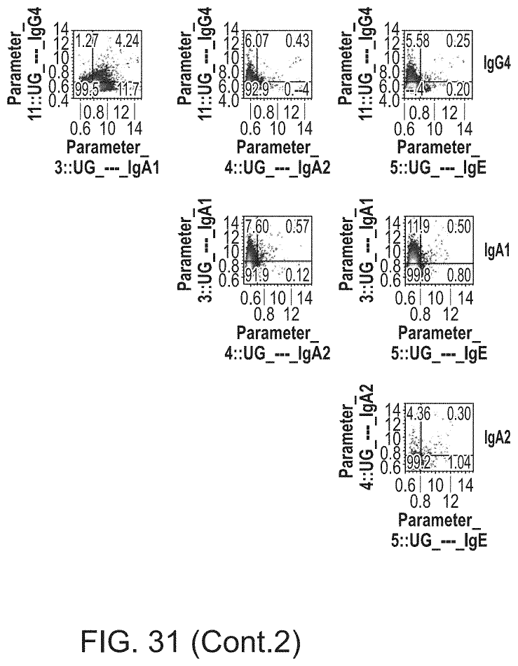

FIG. 31 depicts raw log 2 transformed proteome-wide pan-isotype autoantibody immunoprofiling of a patient with ulcerative colitis. The data demonstrates unique population-wide antigen-specific interrelationships across immunoglobulin isotypes and subtypes associated with ulcerative colitis.

### Example 10: Multi-Isotype Serum Autoantibody Fingerprinting

A human proteome microarray (HuProt Human Proteome Microarray v4.0, CDI Laboratories, Mayaguez, PR) that contains over 21,000 unique, individually-purified full-length human proteins in duplicate, covering more than 81% of the proteome [ref: Jeong J S, Jiang L, Albino E, et al. Rapid identification of monospecific monoclonal antibodies using a human proteome microarray. Mol Cell Proteomics. 2012; 11(6):0111.016253. doi:10.1074/mcp.O111.016253] was used to demonstrate the usefulness of multi-isotype serum autoantibody fingerprinting on a small cohort of healthy control (N=8) and ulcerative colitis case (N=7) pediatric donors for serum autoantibody fingerprinting. Multiple arrays were utilized for each patient in order to cover all possible antibody isotypes and subtypes. Briefly, the HuProt arrays were blocked with blocking solution (5% BSA/1×TBS-T) at room temperature for 1 h, and then probed with serum samples (diluted 1:1000) at 4° C. overnight. The arrays were then washed with 1×TBS-T for 3 times, 10 min each, and probed with up to two anti-human IgG isotype and subtype-specific secondary antibodies (anti-IgG1, anti-IgG2, anti-IgG3, anti-IgG4, anti-IgA, anti-IgM) at room temperature for 1 h, followed by three washes of 1×TBS-T, 10 min each, and then dried with an air duster and scanned using a GenePix 4000B instrument (Molecular Dynamics, Sunnyvale, Calif.). GenePix Pro (v7.2.30) software was used to measure the signal intensities for immunoglobulin binding to array features as well as any background signal present. Array signal intensities data were quality controlled for successful printing, staining, and scanner alignment using internal software tools (CDI Laboratories, Mayaguez, PR) which confirmed duplicate spots retained >0.95 R 2 signal intensity correlation across each array included in this study. Raw data from replicate spot pairs were averaged, log 2 transformed, and grouped. Student's T-tests were used to create P-values for differences in healthy vs ulcerative colitis samples for each autoantigen and each isotype. These P-values were transformed to ABS(LOG 10-P-Value) and plotted against average log 2 signal differences across the cohorts to create volcano plots. The highest P-Value outlier for IgA is TOLLIP (Toll Interacting Protein, a ubiquitin-binding protein that interacts with several Toll-like receptor (TLR) signaling cascade components. The encoded protein regulates inflammatory signaling and is involved in interleukin-1 receptor trafficking and in the turnover of IL1R-associated kinase). For individual patients, antibody isotype ratios were computed for each antigen versus IgA; the largest outlier for ulcerative colitis versus controls enriched for IgA autoantibodies was again TOLLIP. Plotted are individual patient signal ratios for anti-TOLLIP IgA log 2 signal intensity versus the other isotypes (IgG1, IgG2, IgG3, IgG4, IgM). The antibody isotype ratio for anti-TOLLIP autoantibodies is more balanced towards the IgA isotype in ulcerative colitis patients versus healthy controls for all tested isotypes and subtypes (Student's T-test).

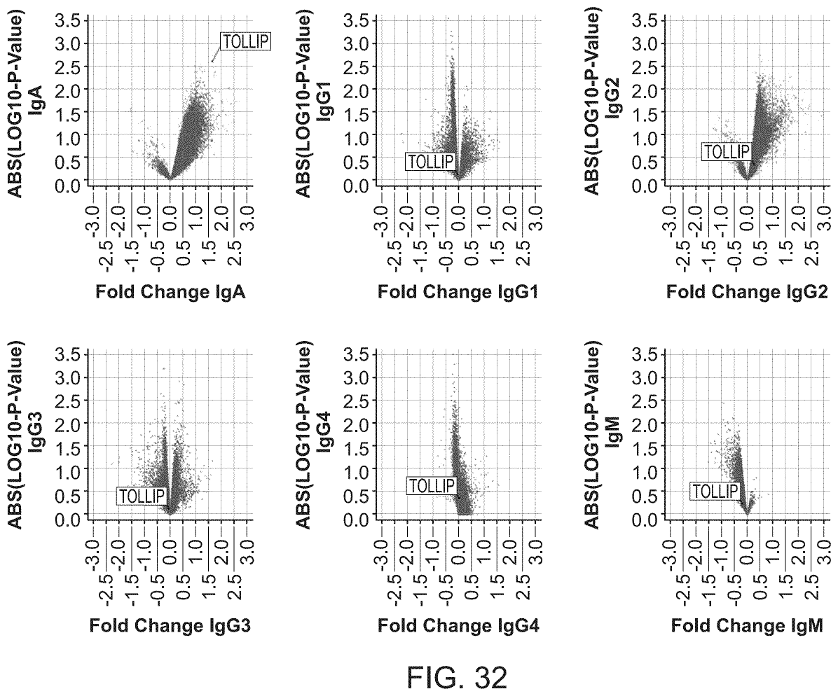

FIG. 32 depicts the utility of this invention via raw log 2 transformed proteome-wide multi-isotype autoantibody immunoprofiling of cohort of patients with ulcerative colitis in comparison with a cohort of healthy patients. Depicted are volcano plots of (X-axis—fold autoantibody signal enrichment in ulcerative colitis in controls; Y-axis—absolute value of the Log 10_P-value by T-test); the protein TOLLIP (Toll Interacting Protein) is the most statistically significant IgA autoantibody differentiator for ulcerative colitis versus healthy controls and autoantibodies to TOLLIP could be the mechanistic cause of ulcerative colitis.

FIG. 33 depicts the utility of this invention utilizing raw log 2 transformed proteome-wide multi-isotype autoantibody immunoprofiling of cohort of patients with ulcerative colitis in comparison with a cohort of healthy patients; the protein TOLLIP (Toll Interacting Protein) is the most statistically significant IgA autoantibody differentiator for ulcerative colitis in this study; this signal is very biased for IgA response versus the other isotypes IgG1, IgG2, IgG3, IgG4, and IgM—more than for any other human protein in the study. TOLLIP is a secreted protein that regulates the activity of cytotoxic neutrophils in the colon in the presence of bacterial components via TLR receptors. Others have reported that low TOLLIP activity could lead to ulcerative colitis. The presence of anti-TOLLIP IgA autoantibodies secreted in the colonic mucosa could block this natural tonic function, creating a permanent mechanistic cause for the chronic condition ulcerative colitis that was previously unknown and was identified by the utility of methods presented herein.

### Example 11: TOLLIP Blocking by IgA Autoantibodies in the Colonic Mucosa

TOLLIP deficiency has been previously reported as a causative agent of ulcerative colitis in mouse models. However, human colonic epithelium from inflammatory bowel disease patients has been reported to be at normal levels (Steenholdt et al., (2009) Expression and function of toll-like receptor 8 and Tollip in colonic epithelial cells from patients with inflammatory bowel disease, Scandinavian Journal of Gastroenterology, 44:2, 195-204, DOI: 10.1080/00365520802495529). Normal levels of colonic TOLLIP, blocked by IgA autoantibodies in the colonic mucosa, would be a way of reconciling these two observations. A deficiency of TOLLIP function could be created by anti-TOLLIP function-blocking autoantibodies in the presence of normal levels of TOLLIP protein. While TOLLIP function is primarily believed to be intracellular, there is abundant evidence for TOLLIP protein in serum and the extracellular space. We propose that TOLLIP is a natural tonic immune signal, sometimes disrupted in the colon by anti-TOLLIP IgA autoantibodies, resulting in the disease ulcerative colitis. To support this theory of TOLLIP as natural immune checkpoint—we investigated TOLLIP expression in patients from The Cancer Genome Atlas (TCGA; accessed via https://www.cbioportal.org/). We first compared the most checkpoint-blockade-susceptible of all tumors—malignant melanoma. Approximately 5% of malignant melanoma patients in this cohort overexpressed TOLLIP mRNA, and their overall survival was significantly worse. This supports the hypothesis that TOLLIP is a natural immune checkpoint, overexpressed by melanoma to protect itself from the adaptive immune system. These data support anti-TOLLIP therapies (monoclonal antibody, DART, BiTE, etc) being a novel checkpoint blockade treatment for malignant melanoma. Additionally, we observed similar results in glioma brain cancer, supporting a similar treatment for other types of cancer.

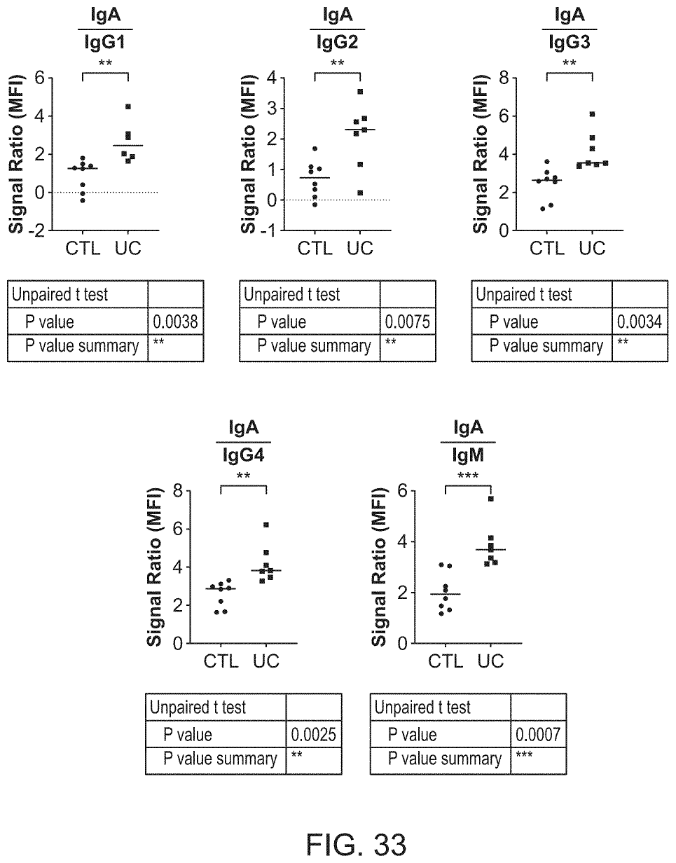

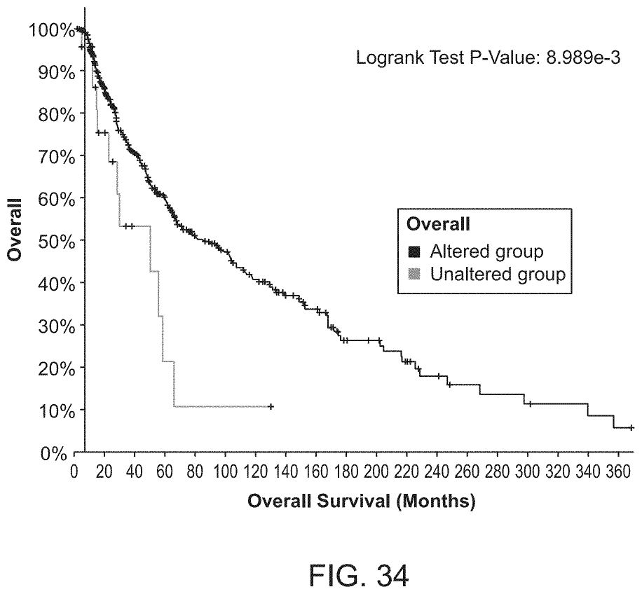

FIG. 34 depicts survival data for cancer patients overexpressing TOLLIP protein having poorer overall survival in The Cancer Genome Atlas (TCGA) metastatic melanoma cohort; these data support TOLLIP being a druggable immune checkpoint—such as might be blocked naturally by natural colonic IgA autoantibodies to cause ulcerative colitis.

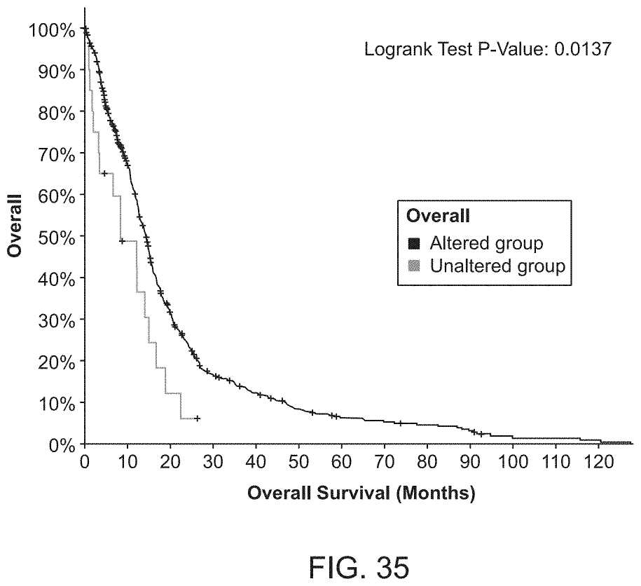

FIG. 35 depicts survival data for cancer patients overexpressing TOLLIP protein having poorer overall survival in The Cancer Genome Atlas (TCGA) glioma brain cancer cohort. these data support TOLLIP being a druggable immune checkpoint—such as might be blocked naturally by natural colonic IgA autoantibodies to cause ulcerative colitis.

### Example 12: Highly Communicable Disease Testing

A microarray is used to determine ratios of autoantibodies in a subject. Based on the ratios the patient is stratified into a risk group, comprising likely to be at low risk of developing symptoms if exposed to a highly communicable disease, likely to have a mild infection, and likely to develop serious complications if exposed to the disease.

In some cases, a highly communicable disease can comprise a respiratory disease. In some cases, a respiratory disease can comprise COVID-19.

1. A method comprising:
(a) contacting an array comprising a protein or peptide with a sample comprising an antibody;
(b) detecting a first antibody isotype and a second antibody isotype in the sample, wherein the first antibody isotype is different to the second antibody isotype; and
(c) computing a ratio of the first antibody isotype to the second antibody isotype.

2. The method of claim 1, wherein the first antibody isotype or the second antibody isotype comprises IgM, B-cell receptor, IgM pentamer, IgG, IgG1, IgG2, IgG3, IgG4, IgA, IgA1, IgA1 monomer, IgA1 dimer, secreted IgA1, dimer, IgA2, IgA2 monomer, secreted IgA2 dimer, IgD, IgD B-cell receptor, IgE, or any combination thereof.

3. The method of claim 2, wherein the first antibody isotype or the second antibody isotype comprises IgG.

4. The method of claim 3, wherein the IgG is of antibody subclass comprising IgG1, IgG2, IgG3 or IgG4.

5. The method of claim 4, wherein the IgG is of an antibody subclass comprising the IgG2, wherein the IgG2 is IgG2A, IgG2B, or IgG2C.

6. The method of claim 1, wherein the array comprises at least one native folded human protein or an antigenic fragment thereof.

7. The method of claim 6, wherein the array comprises at least about 10 native folding human proteins or an antigenic fragment thereof.

8. The method of claim 1, wherein the sample comprises, blood, serum, urine or a combination thereof.

9. The method of claim 8, wherein the sample is from a human subject.

10. The method of claim 9, wherein the array comprises at least about 10,000 unique human proteins.

11. The method of claim 10, wherein the unique human proteins comprise at least 80% full-length proteins.

12. The method of claim 1, wherein the array comprises a virome, allergome, mutanome, microbiome, or phageome.

13. The method of claim 1, wherein the first antibody isotype is an antibody subclass, the second antibody isotype is an antibody subclass, or the first antibody isotype and the second antibody isotype comprises an antibody subclass.

14. The method of claim 1, wherein the detecting of the first antibody isotype and the second antibody isotype in the sample comprises staining of the first antibody isotype and the second antibody isotype.

15. The method of claim 14, wherein the first antibody isotype is stained with a first stain and the second antibody isotype is stained with a different second stain.

16. The method of claim 15, wherein the first stain comprises anti-IgM, anti-IgM B-cell receptor, anti-IgM pentamer, anti-IgG, anti-IgG1, anti-IgG2, anti-IgG3, anti-IgG4, anti-IgM, anti-IgA1 monomer, anti-IgA1 dimer, anti-secreted IgA1 dimer, anti-IgA2, anti-IgA2 monomer, anti-secreted IgA2 dimer, anti-IgD, anti-IgD B-cell receptor, anti-IgE, or any combination thereof.

17. The method of claim 16, wherein the second stain comprises anti-IgM, anti-IgM B-cell receptor, anti-IgM pentamer, anti-IgG, anti-IgG1 anti-IgG2, anti-IgG3, anti-IgG4, anti-IgA, anti-IgA1, anti-IgA1 monomer, anti-IgA1 dimer, anti-secreted IgA1 dimer, anti-IgA2, anti-IgA2 monomer, anti-secreted IgA2 dimer, anti-IgD, anti-IgD B-cell receptor, anti-IgE, or any combination thereof.

18. The method of claim 17, wherein the staining comprises a fluorescent staining, and the detecting comprises detecting the fluorescent staining.

19. The method of claim 18, wherein the detecting the fluorescent staining comprises scanning the array with a scanner.

20. The method of claim 19, wherein the detecting comprises a multicolor detection.

21. The method of claim 20, wherein the detecting comprises a three-dimensional detection.

22. The method of claim 1, wherein the detecting comprises a pull down assay.

23. The method of claim 22, wherein the pull down assay comprises isolating a protein that binds to a bait to produce an isolated protein.

24. The method of claim 23, wherein the detecting further comprises sequencing of the isolated protein.

25. The method of claim 1, wherein the detecting comprises phage immunoprecipitation sequencing (PhIP-seq), Rapid Extracellular Antigen Profiling (REAP), mass spectrometry, protein microarrays, peptide microarrays, Luminex libraries, SEREX, nucleotide barcode labelled peptide sequencing, or any combination thereof.

26. The method of claim 1, wherein a data from the detecting is transmitted to a computer configured to calculate the ratio between the first antibody isotype and the second antibody isotype.

27.-71. (canceled)
